SEQUENCE LISTING (27 sequences)

SEQ ID NO: 1 - DNA, 681 bp, Homo sapiens
gacaaaactcacacatgtccaccttgtccagctccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 579 bp, Homo sapiens
atgggggtgcacgaatgtcctgcctggctgtggcttctcctgtccctgctgtcgctccctctgggcctcccagtcctgggcgccccaccacgcctcatctgtgacagccgagtcctgcagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacaga

SEQ ID NO: 4 - protein, 193 aa, Homo sapiens [sequence omitted]

SEQ ID NO: 5 - DNA, 798 bp, Homo sapiens
ctgcagaccaccatggtaccgtgcacgctgctcctgctgttggcggccgccctggctccgactcagacccgcgccggctctagacccggggaattcgccggcgccgctgcggtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 261 aa, Homo sapiens
MVPCTLLLLLAAALAPTQTRAGSRPGEFAGAAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1290 bp, Homo sapiens
ctgcagaccaccatggtaccgtgcacgctgctcctgctgttggcggccgccctggctccgactcagacccgcgccggctctagagccccaccacgcctcatctgtgacagccgagtcctgcagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagagaattcgccggcgccgctgcggtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 425 aa, Homo sapiens
MVPCTLLLLLAAALAPTQTRAGSRAPPRLICDSRVLQRYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDREFAGAAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1299 bp, Homo sapiens
ctgcaggcggagatgggggtgcacgaatgtcctgcctggctgtggcttctcctgtccctgctgtcgctccctctgggcctcccagtcctgggcgccccaccacgcctcatctgtgacagccgagtcctggagaggtacctcttggaggccaaggaggccgagaatatcacgacgggctgtgctgaacactgcagcttgaatgagaatatcactgtcccagacaccaaagttaatttctatgcctggaagaggatggaggtcgggcagcaggccgtagaagtctggcagggcctggccctgctgtcggaagctgtcctgcggggccaggccctgttggtcaactcttcccagccgtgggagcccctgcagctgcatgtggataaagccgtcagtggccttcgcagcctcaccactctgcttcgggctctgggagcccagaaggaagccatctcccctccagatgcggcctcagctgctccactccgaacaatcactgctgacactttccgcaaactcttccgagtctactccaatttcctccggggaaagctgaagctgtacacaggggaggcctgcaggacaggggacagagaattcgccggcgccgctgcggtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 10 - protein, 428 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDREFAGAAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 11 aa, Homo sapiens
PKNSSMISNTP

SEQ ID NO: 12 - protein, 7 aa, Homo sapiens
HQSLGTQ

SEQ ID NO: 13 - protein, 8 aa, Homo sapiens
HQNLSDGK

SEQ ID NO: 14 - protein, 8 aa, Homo sapiens
HQNISDGK

SEQ ID NO: 15 - protein, 8 aa, Homo sapiens
VISSHLGQ

SEQ ID NO: 16 - protein, 11 aa, Homo sapiens
PKDTLMISRTP

SEQ ID NO: 17 - protein, 16 aa, Homo sapiens
GGSGGSGGGGSGGGGS

SEQ ID NO: 18 - DNA, 31 bp, Artificial sequence
  synthetic oligonucleotide
aaaactgcagaccaccatggtaccgtgcacg

SEQ ID NO: 19 - DNA, 29 bp, Artificial sequence
  synthetic oligonucleotide
cgtctagagccggcgcgggtctgagtcgg

SEQ ID NO: 20 - DNA, 36 bp, Artificial sequence
  synthetic oligonucleotide
aagaattcgccggcgccgctgcggtcgacaaaactc

SEQ ID NO: 21 - DNA, 28 bp, Artificial sequence
  synthetic oligonucleotide
ttcaattgtcatttacccggagacaggg

SEQ ID NO: 22 - DNA, 32 bp, Artificial sequence
  synthetic oligonucleotide
aatctagagccccaccacgcctcatctgtgac

SEQ ID NO: 23 - DNA, 30 bp, Artificial sequence
  synthetic oligonucleotide
ttgaattctctgtcccctgtcctgcaggcc

SEQ ID NO: 24 - DNA, 27 bp, Artificial sequence
  synthetic oligonucleotide
gtacctgcaggcggagatgggggtgca

SEQ ID NO: 25 - DNA, 22 bp, Artificial sequence
  synthetic oligonucleotide
cctggtcatctgtcccctgtcc

SEQ ID NO: 26 - protein, 13 aa, Artificial sequence
  synthetic peptide
GSRPGEFAGAAAV

SEQ ID NO: 27 - protein, 8 aa, Artificial sequence
  synthetic peptide
EFAGAAAV

(19) United States
(12) Patent Application Publication
(10) Pub. No.: US 2003/0235536 A1
(43) Pub. Date: Dec. 25, 2003

(54) Central airway administration for systemic delivery of therapeutics

(75) Inventors: Richard S. Blumberg, Chestnut Hill, MA (US); Wayne I. Lencer, Jamaica Plain, MA (US); Neil E. Simister, Wellesley, MA (US); Alan J. Bitonti, Acton, MA (US)

Correspondence Address: WOLF GREENFIELD & SACKS, PC, FEDERAL RESERVE PLAZA, 600 ATLANTIC AVENUE, BOSTON, MA, 02210-2211, US

(73) Assignees: The Brigham and Women's Hospital, Inc., Boston, MA (US); Children's Medical Center Corporation, Boston, MA (US); Brandeis University, Waltham, MA (US); Syntonix Pharmaceuticals, Inc., Waltham, MA (US)

(21) Appl. No.: 10/435,608

(22) Filed: May 9, 2003

Related U.S. Application Data
(63) Continuation-in-part of application No. PCT/US02/21335, filed on Jul. 3, 2002.
(60) Provisional application No. 60/364,482, filed on Mar. 15, 2002.

(51) Int. Cl. A61L 9/04; A61K 48/00; A61K 38/23; A61K 38/21

(52) U.S. Cl. 424/45; 514/2; 514/44; 424/85.5; 424/85.6; 424/85.7

(57) ABSTRACT

The present invention relates to methods and products for the transepithelial systemic delivery of therapeutics. In particular, the invention relates to methods and compositions for the systemic delivery of therapeutics by administering an aerosol containing antibodies or conjugates of a therapeutic agent with an FcRn binding partner to epithelium of central airways of the lung. The methods and products are adaptable to a wide range of therapeutic agents, including proteins and polypeptides, nucleic acids, drugs, and others. The methods and products have the advantage of not requiring administration to the deep lung in order to effect systemic delivery.

### RELATED APPLICATIONS

This application is a continuation-in-part of international patent application PCT/US02/21355, filed Jul. 3, 2002, designating the United States and now pending, which in turn claims benefit of U.S. provisional patent application 60/364,482, filed Mar. 15, 2002, now expired.

### FIELD OF THE INVENTION

The present invention relates to methods and products for the transepithelial systemic delivery of therapeutics. In particular, the invention relates to methods and compositions for the systemic delivery of therapeutics which include a neonatal Fc receptor (FcRn) binding partner by their administration to central airways of the lung. Such therapeutics include therapeutic and diagnostic IgG antibodies as well as conjugates formed between a therapeutic agent and an FcRn binding partner. The methods and compositions are useful for any indication for which the therapeutic is itself useful in the detection, treatment or prevention of a disease, disorder, or other condition of a subject.

### BACKGROUND OF THE INVENTION

Transport of macromolecules across an epithelial barrier can occur by receptor-nonspecific or receptor-specific mechanisms. Receptor-nonspecific mechanisms are represented by paracellular sieving events, the efficiency of which are inversely related to the molecular weight of the transported molecule. Transport of macromolecules such as immunoglobulin G (IgG) via this paracellular pathway is highly inefficient due to the large molecular mass of IgG (ca. 150 kDa). Receptor-nonspecific transport may include transcytosis in the fluid phase. This is much less efficient than receptor-mediated transport, because most macromolecules in the fluid phase are sorted to lysosomes for degradation. In contrast, receptor-specific mechanisms which may provide highly efficient transport of molecules otherwise effectively excluded by paracellular sieving. Such receptor-mediated mechanisms may be understood teleologically as effective scavenger mechanisms for anabolically expensive macromolecules such as albumin, transferrin, and immunoglobulin. These and other macromolecules would otherwise be lost at epithelial barriers through their diffusion down an infinite concentration gradient from inside to outside the body. Receptor-specific mechanisms for transport of macromolecules across epithelia exist for only a few macromolecules.

The surfaces defining the boundary between the inside of the body and the external world are provided by specialized tissue called epithelium. In its simplest form, epithelium is a single layer of cells of a single type, forming a covering of an external or “internal” surface. Epithelial tissues arise from endoderm and ectoderm and thus include skin, epithelium of the cornea (eye), as well as the “internal” lining surfaces of the gastrointestinal tract, genitourinary tract, and respiratory system. These “internal” lining surfaces communicate with the external world, and thus they form a boundary between the inside of the body and the external world. While these various epithelia have specialized structural features or appendages that distinguish them, they also share much in common.

Two features common among various epithelia are the combination of large surface area on a gross level and close apposition with tight junctions on a cellular level. These two features present potential advantages and disadvantages, respectively, for the use of epithelium as a site for systemic, non-invasive delivery of therapeutics. For example, the surface area of the lung epithelium in human adults is believed to be 140 m². This enormous surface therefore potentially presents a highly attractive site of administration for systemic delivery of therapeutic agents, provided, of course, the therapeutic agent can be delivered to the epithelium and then transported across the epithelium.

Yet a third feature characteristic of various epithelia, and of particular importance to the present invention, is the receptor-specific mechanism for transport across an epithelial barrier provided by FcRn (neonatal Fc receptor). This receptor was first identified in neonatal rat and mouse intestinal epithelia and shown to mediate transport of maternal IgG from milk to the blood-stream of the suckling rat or mouse. IgG transferred to the neonate by this mechanism is critical for immunologic defense of the newborn. Expression of FcRn in rat and mouse intestinal epithelia was reported to cease following the neonatal period. In humans, humoral immunity does not depend on neonatal intestinal IgG transport. Rather, it was believed that a receptor of the placental tissue was responsible for IgG transport. The receptor responsible for this transport had been sought for many years. Several IgG-binding proteins had been isolated from placenta. FcγRII was detected in placental endothelium and FcγRIII in syncytiotrophoblasts. Both of these receptors, however, showed a relatively low affinity for monomeric IgG. In 1994, Simister and colleagues reported the isolation from human placenta of a cDNA encoding a human homolog of the rat and mouse Fc receptor for IgG. Story CM et al. (1994) J Exp Med 180:2377-81. The complete nucleotide and deduced amino acid sequences were reported and are publicly available as GenBank Accession Nos. U12255 and AAA58958, respectively.

Unlike the rodent intestinal FcRn, the human FcRn was unexpectedly discovered to be expressed in adult epithelial tissues. U.S. Pat. Nos. 6,030,613 and 6,086,875. Specifically, human FcRn was found to be expressed on lung epithelial tissue, as well as on intestinal epithelial tissue (Israel E J et al. (1997) Immunology 92:69-74), renal proximal tubular epithelial cells (Kobayashi N et al. (2002) Am J Physiol Renal Physiol 282:F358-65), and other mucosal epithelial surfaces including nasal epithlium, vaginal surfaces, and biliary tree surfaces.

U.S. Pat. No. 6,030,613, issued to Blumberg et al., discloses methods and compositions for the delivery of therapeutics conjugated to an FcRn binding partner to intestinal epithelium, mucosal epithelium, and epithelium of the lung.

U.S. Pat. No. 6,086,875, also issued to Blumberg et al., discloses methods and compositions for stimulating an immune response to an antigen by the delivery of an antigen conjugated to an FcRn binding partner to an FcRn-expressing epithelium, including epithelium of the lung.

It is widely believed that administration of a therapeutic to lung epithelium for systemic delivery of the therapeutic requires delivery to the deep lung, i.e., to periphery of the lung, because that is how to access the greatest amount of surface area available. Yu J et al. (1997) Crit Rev Therapeutic Drug Carrier Systems 14:395-453. In addition, the epithelium lining the deepest reaches of the lungs, the alveoli, is a monolayer of extremely thin cells. In contrast, the epithelium of more proximal airways of the lungs are considerably thicker, and they are equipped with cilia to facilitate clearance of materials that could otherwise accumulate in the more distal airways and alveoli and thereby interfere with gas exchange. Aerosol delivery systems and methods therefore have been developed with the goal of maximizing drug delivery to the deep lung. This typically requires a combination of factors related both to the aerosol generator, e.g., metered dose inhaler (MDI) device, and special inhalation techniques to be employed by the patient in using the aerosol generator. For example, a typical MDI may be designed to generate the smallest possible droplets or particles, and it may be fitted for use with a spacer device or attachment to trap and remove larger, lower-velocity particles from the aerosol. The user may typically have to coordinate discharge of the MDI with initiation of inspiration, rate and depth of inspiration, breath-holding, and the like, all in order to increase the likelihood of effective delivery of the active agent to the deepest reaches of the lungs. Needless to say, patient compliance and therapeutic efficacy are frequently compromised by these technical requirements.

### SUMMARY OF THE INVENTION

The present invention relates in part to the surprising discovery by the inventors that expression of FcRn on pulmonary epithelium is more extensive in central airways than in the periphery of the lung. This density distribution of FcRn in pulmonary epithelium actually favors aerosol administration of a therapeutic agent to central airways, rather than to deep lung, when the therapeutic agent includes or incorporates an FcRn binding partner. It has been discovered according to the present invention that administration of aerosolized FcRn binding partner conjugate to central airways permits highly efficient FcRn-mediated transcytosis of the conjugate across the respiratory epithelium and systemic delivery of the therapeutic agent. Unlike other methods and compositions for systemic delivery via pulmonary administration, the invention advantageously requires no special breathing techniques to effect systemic delivery. The technical obstacles presented by the need for deep lung delivery are thereby averted, and the invention provides effective strategies useful for noninvasive, systemic delivery of a therapeutic agent to a subject through its aerosol administration to central airways of the lung as a conjugate with an FcRn binding partner.

It has now been discovered according to the instant invention that systemic delivery of an antibody to a subject can be achieved by directing administration of the antibody to a central airway of the subject. The methods of the invention thus exploit the predominance of FcRn receptor expression in central airways of the lung to mediate highly efficient transcellular transport of an antibody across pulmonary epithelium to effect systemic delivery of the antibody.

The invention is useful wherever it is desirable to achieve systemic delivery of therapeutics, including antibodies. The invention is useful, for example, wherever it is desirable to administer a particular therapeutic agent to a subject for the treatment or prevention of a condition of the subject that is treatable with the therapeutic agent. The invention can be particularly useful whenever repeated or chronic administration of a therapeutic agent is called for, compliance with a special breathing technique is difficult to achieve, as well as whenever it is desirable to avoid invasive administration.

According to one aspect of the invention, a method for systemic delivery of a therapeutic agent is provided. The method involves administering an effective amount of an aerosol of a conjugate of a therapeutic agent and an FcRn binding partner to lung such that a central lung zone/peripheral lung zone deposition ratio (C/P ratio) is at least 0.7. As explained further below, the C/P ratio is selected such that the conjugate is intentionally delivered to central airways.

The C/P ratio in one embodiment according to this aspect of the invention is at least 1.0. In another embodiment the C/P ratio is at least 1.5. In another embodiment the C/P ratio is at least 2.0. In another embodiment the C/P ratio is at least 3.0.

Important to this and other aspects of the invention, in one embodiment the administering to a central airway of the subject involves tidal breathing by the subject. In this regard the methods of the invention represent a marked departure from the current focus on alveolar administration in all other methods directed to pulmonary administration of macromolecules such as antibodies. For example, the breathing techniques useful according to the invention do not require breath holding, deeper-than-normal inhalation, or special timing. The methods are thus particularly useful where such maneuvers are difficult to achieve, e.g., due to age (e.g., neonates and infants) or coordination of the subject.

According to another aspect of the invention, a method is provided for systemic delivery of a therapeutic agent. The method involves administering an effective amount of an aerosol of a conjugate of a therapeutic agent and an FcRn binding partner to lung, wherein particles in the aerosol have a mass median aerodynamic diameter (MMAD) of at least 3 micrometers (μm).

According to yet another aspect, the invention provides an aerosol of a conjugate of a therapeutic agent and an FcRn binding partner, wherein particles in the aerosol have a MMAD of at least 3 μm.

According to still another aspect, the invention provides an aerosol delivery system. The aerosol delivery system according to this aspect includes a container, an aerosol generator connected to the container, and a conjugate of a therapeutic agent and an FcRn binding partner disposed within the container, wherein the aerosol generator is constructed and arranged to generate an aerosol of the conjugate having particles with a MMAD of at least 3 μm.

In one embodiment, this aspect provides a method of manufacturing the aerosol delivery system. The method involves the steps of providing the container, providing the aerosol generator connected to the container, and placing an effective amount of the conjugate in the container.

In some embodiments according to this aspect of the invention, the aerosol generator includes a vibrational element in fluid connection with a solution containing the conjugate.

In some embodiments, the vibrational element comprises a member having (a) a front surface; (b) a back surface in fluid connection with the solution; and (c) a plurality of apertures traversing the member. In certain embodiments, the apertures at the front surface are at least 3 μm in diameter. The apertures can be tapered so that they narrow from the back surface to the front surface.

In some embodiments according to this aspect of the invention, the aerosol generator is a nebulizer. In some embodiments, the nebulizer is a jet nebulizer.

In some embodiments according to this aspect of the invention, the aerosol generator is a mechanical pump.

In some embodiments according to this aspect of the invention, the container is a pressurized container.

According to still another aspect, the invention provides an aerosol delivery system. The aerosol delivery system according to this aspect includes a container, an aerosol generator connected to the container, and a conjugate of a therapeutic agent and an FcRn binding partner disposed within the container, wherein the aerosol generator includes a means for generating an aerosol of the conjugate having particles with a MMAD of at least 3 μm.

In one embodiment, this aspect provides a method of manufacturing the aerosol delivery system. The method involves the steps of providing the container, providing the aerosol generator connected to the container, and placing an effective amount of the conjugate in the container.

In some embodiments according to this aspect of the invention, the aerosol generator includes a vibrational element in fluid connection with a solution containing the conjugate.

In some embodiments, the vibrational element comprises a member having (a) a front surface; (b) a back surface in fluid connection with the solution; and (c) a plurality of apertures traversing the member. In certain embodiments, the apertures at the front surface are at least 3 μm in diameter. The apertures can be tapered so that they narrow from the back surface to the front surface.

In some embodiments according to this aspect of the invention, the aerosol generator is a nebulizer. In some embodiments, the nebulizer is a jet nebulizer.

In some embodiments according to this aspect of the invention, the aerosol generator is a mechanical pump.

In some embodiments according to this aspect of the invention, the container is a pressurized container.

In each of the foregoing aspects of the invention, in some embodiments the MMAD of the particles is between 3 μm and about 8 μm. In some embodiments the MMAD of the particles is greater than 4 μm. In certain embodiments a majority of the particles are non-respirable, i.e., they have a MMAD of at least 4.8 μm. Non-respirable particles are characterized as substantially unable to enter the alveolar space in the deep lung.

In each of the foregoing aspects of the invention, in some embodiments the FcRn binding partner contains a ligand for FcRn which mimics that portion of the Fc domain of IgG which binds the FcRn (i.e., an Fc, an Fc domain, Fc fragment, Fc fragment homolog). In certain embodiments, the FcRn binding partner is non-specific IgG or an FcRn-binding fragment of IgG. Most typically the FcRn binding partner corresponds to the Fc fragment of IgG, i.e., Fcγ. The Fcγ can be native or it can be modified so that it has a higher affinity for FcRn than native Fcγ. Such modification can include substitution of certain amino acid residues involved in contact with FcRn. The Fcγ can be modified so that it has a longer circulating half-life than native Fcγ. Such modification can include substitution of certain amino acid residues involved in interaction with Fc receptors other than FcRn, substitution of certain amino acid residues involved in glycosylation, and the like.

In each of the foregoing aspects of the invention, in some embodiments the therapeutic agent and the FcRn binding partner are coupled by a covalent bond.

In each of the foregoing aspects of the invention, in some embodiments the therapeutic agent and the FcRn binding partner are coupled by a linker. In certain embodiments the linker is a peptide linker. In some embodiments the linker includes at least part of a substrate for an enzyme that specifically cleaves the substrate.

In each of the foregoing aspects of the invention, in some embodiments the therapeutic agent is a polypeptide. The conjugate in such embodiments can be an isolated fusion protein. In certain such embodiments, the polypeptide therapeutic agent of the conjugate can be linked to the FcRn binding partner by a linker, provided the polypeptide therapeutic agent and the FcRn binding partner each retains at least some of its biological activity.

In certain embodiments the polypeptide that is conjugated with the FcRn binding partner includes an antigen-specific antibody fragment. In certain embodiments the polypeptide that is conjugated with the FcRn binding partner is an antigen-specific antibody fragment. The antigen-specific antibody fragment can be a Fab, F(ab′), F(ab′)₂, Fv, or single chain Fv. In certain embodiments the antigen-specific antibody fragment is further conjugated with an antigen to which it specifically binds.

In each of the foregoing aspects of the invention, in some embodiments the therapeutic agent is a cytokine. In some embodiments the therapeutic agent is a cytokine receptor or a cytokine-binding fragment thereof.

In each of the foregoing aspects of the invention, in some embodiments the therapeutic agent is an antigen. The antigen can be characteristic of a pathogen, characteristic of an autoimmune disease, characteristic of an allergen, or characteristic of a tumor. In certain embodiments the antigen is a tumor antigen.

In each of the foregoing aspects of the invention, in some embodiments the therapeutic agent is an oligonucleotide. In certain embodiments the oligonucleotide is an antisense oligonucleotide.

In each of the foregoing aspects of the invention, in some embodiments the therapeutic agent is erythropoietin (EPO), growth hormone, interferon alpha (IFN-α), interferon beta (IFN-β), or follicle stimulating hormone (FSH). In each of the foregoing aspects of the invention, in some embodiments the therapeutic agent is Factor VIIa, Factor VIII, Factor IX, tumor necrosis factor-alpha (TNF-α), TNF-α receptor (for example, etanercept, ENBREL®; see U.S. Pat. No. 5,605,690, PCT/US93/08666 (WO 94/06476), and PCT/US90/04001 (WO 91/03553)), lymphocyte function antigen-3 (LFA-3), or ciliary neurotrophic factor (CNTF). In each and every one of these and like embodiments, the therapeutic agent is a biologically active polypeptide, whether whole or a portion thereof. For example, a therapeutic agent that is a TNF receptor (TNFR) includes whole TNFR as well as a TNF-binding TNF receptor polypeptide, e.g., an extracellular domain of TNFR.

In one aspect the invention provides a method for systemic delivery of an antibody to a subject. The method involves administering to a central airway of a subject an antibody in an aerosol, wherein a central lung zone/peripheral lung zone deposition ratio (C/P ratio) is at least 0.7, in an effective amount to achieve systemic delivery of the antibody to the subject. In independent and individual embodiments the C/P ratio of the antibody can be at least: 0.8; 0.9; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9; or 3.0. While in theory there is no upper limit of the C/P ratio, in typical usage the C/P ratio will be 100 or less. According to this and other aspects of the invention, in one embodiment the C/P ratio is at least 1.0. In one embodiment the C/P ratio is at least 1.5. In one embodiment the C/P ratio is at least 2.0. In one embodiment the C/P ratio is at least 3.0. In yet another embodiment the C/P ratio is at least 4.0. Also according to this aspect of the invention, in one embodiment the systemic delivery is a peak serum concentration of the antibody of at least 0.5 μg/ml. In one embodiment the systemic delivery is a peak serum concentration of the antibody of at least twice a therapeutically effective concentration of the antibody. The administering can involve a single administration or it can involve more than one administration.

In one embodiment the antibody has an FcRn binding domain. In one embodiment according to this and other aspects of the invention, the antibody has a human Fc fragment. Further, in one embodiment according to this aspect and all aspects of the invention, the antibody has a human IgG1 Fc fragment, i.e., human Fcγ1.

Also according to this and other aspects of the invention, in one embodiment the antibody is a monoclonal antibody. Monoclonal antibodies according to this and other aspects of the invention include both conventional and so-called engineered antibodies. Specifically, engineered antibodies include chimeric antibodies, humanized antibodies, and certain human antibodies. In an alternative embodiment according to this and other aspects of the invention, the antibody is an immune globulin or a hyperimmune globulin.

In this and other aspects of the invention, the antibody can be a therapeutic antibody or a diagnostic antibody. The therapeutic antibody according to certain embodiments is chosen from anti-CD52, anti-CD25, anti-TNF-α, anti-RSV, anti-CD20, anti-HER2, anti-CEA. Specific embodiments of such therapeutic antibodies include CAMPATH®, SIMULECT®, ZENAPAX®, REMICADE®, HUMIRA™, SYNAGIS®, RITUXAN®, HERCEPTIN®, and CEA-CIDE™. Additional antibodies useful according to the invention include but are not limited to RAPTIVA™ (efalizumab, XOMA/Genentech), ZEVALIN™ (ibritumomab tiuxetan, IDEC), BEXXAR® (tositumomab, Corixa), pexulizamab, eculizamab, Oncolym, PRO 542, PRO 140, COTARA™ (Peregrine), ABX-EGF, and MDX-010. The therapeutic antibody according to this aspect and other aspects of the invention can optionally be linked to a cytotoxic agent selected from a radionuclide and a toxin.

In one embodiment according to this aspect of the invention, the antibody is a diagnostic antibody. The diagnostic antibody according to this aspect and other aspects of the invention can in one embodiment be a diagnostic imaging antibody, e.g., an antibody linked to a radionuclide, a metal, a fluorophore, a chromogen, biotin, or other suitable tag useful for detecting the antibody.

Also important to this and other aspects of the invention, in one embodiment the aerosol is composed of predominantly non-respirable particles. In typical usage such non-respirable particles will have a MMAD of at least 4.8 μm. While in theory there is no upper limit of the non-respirable particle size, in typical usage the non-respirable particles will have a MMAD of about 5 μm to 50 μm. In one embodiment the non-respirable particles will have a MMAD of about 5 μm to 20 μm. In one embodiment the non-respirable particles will have a MMAD of about 5 μm to 10 μm.

In another aspect the invention provides a method for passively immunizing a subject. The method according to this aspect of the invention involves administering to a central airway of a subject, wherein said subject is in need of passive immunization against an antigen, an antigen-specific antibody in an aerosol, wherein a C/P ratio is at least 0.7, in an effective amount to neutralize the antigen in the subject.

In yet another aspect the invention provides a method for treating a deep lung disease in a subject. The method according to this aspect of the invention involves administering to a central airway of a subject, wherein said subject is in need of an antibody for treatment of a deep lung disease, an antibody in an aerosol, wherein a C/P ratio is at least 0.7, in an effective amount to treat the deep lung disease of the subject. In certain embodiments the deep lung disease is any one of RSV pneumonia, CMV pneumonia, primary lung cancer, extranodal pulmonary non-Hodgkin's lymphoma, and cancer metastatic to lung. Accordingly, in certain embodiments the antibody is any one of anti-RSV, anti-CMV, anti-CD52, anti-CD20, anti-HER2, and anti-CEA. In particular embodiments the antibody is any one of SYNAGIS®, CAMPATH®, RITUXAN®, HERCEPTIN®, and CEA-CIDE™. For example, where the deep lung disease is RSV pneumonia, the antibody can be SYNAGIS®. Where the deep lung disease is CMV pneumonia, the antibody can be CYTOGAM®. Where the deep lung disease is extranodal pulmonary non-Hodgkin's lymphoma, the antibody can be CAMPATH® or RITUXAN®. Where the deep lung disease is cancer metastatic to lung, the antibody can be HERCEPTIN® or CEA-CIDE™.

In another aspect the invention provides a method for treating extrapulmonary disease in a subject. The method according to this aspect of the invention involves administering to a central airway of a subject, wherein said subject is in need of an antibody for treatment of extrapulmonary disease, an antibody in an aerosol, wherein a central lung zone/peripheral lung zone deposition ratio (C/P ratio) is at least 0.7, in an effective amount to treat the extrapulmonary disease of the subject. In one embodiment the extrapulmonary disease is cancer. Where the extrapulmonary disease is cancer, in certain embodiments the antibody is chosen from anti-CD52, anti-CD25, anti-CD20, anti-HER2, and anti-CEA. In particular, in certain embodiments the antibody is chosen from CAMPATH®, SIMULECT®, ZENAPAX®, RITUXAN®, HERCEPTIN®, and CEA-CIDE™.

In another embodiment according to this aspect of the invention, the extrapulmonary disease is an autoimmune disease. In one embodiment the autoimmune disease is rheumatoid arthritis; in another embodiment the autoimmune disease is Crohn's disease. Where the extrapulmonary disease is an autoimmune disease, in one embodiment the antibody is anti-TNF-α. In a particular embodiment, the antibody is REMICADE®. In another particular embodiment, the antibody is HUMIRA™.

In another embodiment according to this aspect of the invention, the extrapulmonary disease is non-pulmonary allograft rejection. Where the extrapulmonary disease is non-pulmonary allograft rejection, in one embodiment the antibody is anti-CD25. In a particular embodiment, the antibody is selected from SIMULECT® and ZENAPAX®.

In each of the foregoing aspects of the invention, in certain embodiments the conjugate or antibody, as delivered to a central airway, is substantially in its native, non-denatured form. In various embodiments at least 60 percent, at least 70 percent, at least 80 percent, at least 90 percent, or at least 95 percent of the conjugate or antibody is in its native, non-denatured form.

These and other aspects of the invention are described in greater detail below.

### BRIEF DESCRIPTION OF THE FIGURES

FIG. 1 presents nucleotide (SEQ ID NO: 1) and amino acid (SEQ ID NO: 2) sequences of human IgG1 Fc fragment (Fcγ1) including the hinge, C_{H}2, and C_{H}3 domains. Numbers beneath the amino acid sequence correspond to the amino acid designations using the EU numbering convention.

FIG. 2 presents cDNA open reading frame nucleotide (panel A; SEQ ID NO: 3) and deduced amino acid (panel B; SEQ ID NO: 4) sequences of wildtype human EPO. The signal peptide in SEQ ID NO: 4 is underlined.

FIG. 3 presents a plasmid map for expression plasmid pED.dC.XFc (panel A) and the nucleotide (SEQ ID NO: 5) and amino acid (SEQ ID NO: 6) sequences of the K^{b} signal peptide/Fcγ1 insert (panel B). The K^{b} signal peptide and the Fcγ1 regions are indicated by a tilde (˜) above the sequence. The EcoRI, PstI and XbaI restriction enzyme sites are underlined.

FIG. 4 presents a plasmid map for expression plasmid pED.dC.EpoFc (panel A) and the nucleotide (SEQ ID NO: 7) and amino acid (SEQ ID NO: 8) sequences of the K^{b} signal peptide/EPO/Fcγ1 insert (panel B). The K^{b} signal peptide, mature EPO, and Fcγ1 regions are indicated by a tilde (˜) above the sequence. The EcoRI, SbfI and XbaI restriction enzyme sites are underlined.

FIG. 5 presents a plasmid map for expression plasmid pED.dC.natEpoFc (panel A) and the nucleotide (SEQ ID NO: 9) and amino acid (SEQ ID NO: 10) sequences of the nativeEPO/Fcγ1 insert (panel B). The mature EPO, including the native EPO signal peptide, and Fcγ1 regions are indicated by a tilde (˜) above the sequence. The EcoRI, PstI and XbaI restriction enzyme sites are underlined.

FIG. 6 is a pair of graphs depicting in vivo response to EPO-Fc administered as an aerosol to central airways of cynomolgus monkeys. Panel A shows maximum reticulocyte response for each of nine animals. Aerosolized EPO-Fc was administered to spontaneously breathing animals using a nebulizer. Panel B shows the maximum serum concentration of EPO-Fc (native Fc fragment) and mutant EPO-Fc (Fc fragment having mutations of three amino acids critical for FcRn binding) following inhalation by shallow or deep breathing.

FIG. 7 is a graph depicting the maximum serum concentration of EPO-Fc in cynomolgus monkeys following aerosol administration at 20% vital capacity (20% VC, shallow breathing) and 75% vital capacity (75% VC, deep breathing).

FIG. 8 is a graph depicting serum concentration over time of EPO-Fc in cynomolgus monkeys following aerosol administration at 20% vital capacity at doses of 30 μg/kg (circles) and 10 μg/kg (triangles). Each curve represents data from a single animal.

FIG. 9 is a graph depicting serum concentration over time of IFN-α-Fc or IFN-α alone in cynomolgus monkeys following aerosol administration of IFN-α-Fc or INTRON® A using shallow breathing at doses of 20 μg/kg. Each curve represents data from a single animal.

FIG. 10 is a graph depicting serum concentration over time of IFN-α-Fc in cynomolgus monkeys following aerosol administration of IFN-α-Fc using shallow breathing at doses of 2 μg/kg. Each curve represents data from a single animal.

FIG. 11 is a pair of graphs depicting oligoadenylate synthetase (OAS) activity (panel A) and neopterin concentration (panel B), two common measures of IFN-α bioactivity, following aerosol administration of IFN-α-Fc using shallow breathing at doses of 20 μg/kg. Each curve represents data from a single animal.

FIG. 12 is a graph depicting serum concentration over time of ENBREL® (human TNFR-Fc) in cynomolgus monkeys following aerosol administration of IFN-α-Fc using shallow breathing at estimated deposited doses of 0.3-0.5 mg/kg. Each curve represents data from a single animal.

FIG. 13 is a pair of graphs depicting serum concentrations over time of biotinylated SYNAGIS® (fully humanized monoclonal anti-RSV antibody) in cynomolgus monkeys following aerosol administration using shallow breathing (panel A) or deep breathing (panel B). Each animal received an estimated deposited dose of 0.6 mg/kg. Each curve represents data from a single animal.

### DETAILED DESCRIPTION OF THE INVENTION

The invention is useful whenever it is desirable to deliver a therapeutic agent across lung epithelium to effect systemic delivery of the therapeutic agent. Advantageously, the invention can be used in the systemic delivery of therapeutics of nearly any size, including those having very large molecular weight. In certain featured aspects of the invention a therapeutic agent is administered to central airways as a conjugate with an FcRn binding partner, where the central airways are by nature peculiarly suited for FcRn receptor-mediated transcellular transport of FcRn binding partners. Also in certain featured aspects of the invention, an antibody having an FcRn binding partner is administered to central airways, where the central airways are by nature peculiarly suited for FcRn receptor-mediated transcellular transport of IgG and other Fcγ-containing antibodies. The invention in certain aspects thus can be used for the pulmonary administration and systemic delivery of IgG antibodies and conjugates of an FcRn binding partner and an agent selected from macromolecules, peptides, oligonucleotides, small molecules, drugs, and diagnostic agents.

Notably, the methods of the invention differ sharply from the current trend in pulmonary delivery, which is devoted to achieving deep lung administration of aerosols of polypeptides and other macromolecules. The methods of the instant invention involve pulmonary administration that is substantially directed to central airways of the lung. It has been discovered by the applicants of the instant invention that FcRn is expressed preferentially in the central airways of the lung, rather than in the deep lung. Thus methods of the invention, which seek to take advantage of FcRn-receptor-mediated transcellular transport, are based in part on administering FcRn-transportable molecules to this central airway region of the lung where FcRn expression is most pronounced. Importantly, the methods obviate the need for deep lung administration and thus overcome difficulties associated with achieving deep lung administration. The invention can be used to achieve systemic delivery of a wide variety of therapeutics, encompassing both therapeutic and diagnostic agents, using a simple and noninvasive method of administration.

As used herein, a “central airway” refers to a conducting or transitional airway, distal to the larynx, which has little to no role in gas exchange. In humans central airways include the trachea, main bronchi, lobar bronchi, segmental bronchi, small bronchi, bronchioles, terminal bronchioles, and respiratory bronchioles. The central airways thus account for the first 16-19 generations of airway branching in the lung, where the trachea is generation zero (0) and the alveolar sac is generation 23. Wiebel E R (1963) Morphometry of the Human Lung, Berlin:Springer-Verlag, pp. 1-151. The central airways are responsible for the bulk movement of air, as opposed to the periphery of the lung, which is primarily responsible for gas exchange between air and blood. In one embodiment the central airways include the first sixteen generations of airway branching. In one embodiment the central airways include the first sixteen generations of airway branching. In one embodiment the central airways include the first seventeen generations of airway branching. In one embodiment the central airways include the first eighteen generations of airway branching. In one embodiment the central airways include the first nineteen generations of airway branching. In aggregate, the central airways account for only about ten percent of the entire respiratory epithelial surface area of the lungs. Qiu Y et al. (1997) In: Inhalation Delivery of Therapeutic Peptides and Proteins, Adjei A L and Gupta P K, eds., Lung Biology in Health and Disease, Vol. 107, Marcel Dekker: New York, pp. 89-131.

As used herein, the terms “periphery of the lung” and, equivalently, “deep lung” refer generally to airways of the lung distal to the central airways. As discussed further below, administration of therapeutics to the deep lung involves overcoming a number of physical and physiological barriers which together serve to protect the integrity of the gas-exchange mechanisms of the lung.

Notably, epithelial cell types vary between the central and peripheral regions of the lung. Central airways are lined by ciliated columnar epithelial cells and cuboidal epithelial cells, whereas the respiratory zone is lined by cuboidal epithelial cells and, more distally, alveolar epithelial cells. Whereas the distance across alveolar epithelium is very small, i.e., 0.1-0.2 μm, the distance across columnar and cuboidal epithelial cells is many times greater, e.g., 30-40 μm for columnar epithelium.

An “aerosol” as used herein refers to a suspension of liquid or solid in the form of fine particles dispersed in a gas. As used herein, the term “particle” thus refers to liquids, e.g., droplets, and solids, e.g., powders. Pharmaceutical aerosols for the systemic delivery of conjugates of the invention to the lungs are, in one embodiment, inhaled via the mouth, and not via the nose. Alternatively, pharmaceutical aerosols for the delivery of conjugates of the invention to the lungs are, in one embodiment, introduced through direct delivery to a central airway, for example via an endotracheal tube or tracheotomy tube. In like manner, pharmaceutical aerosols for the systemic delivery of antibodies to the lungs are, in one embodiment, inhaled via the mouth, and not via the nose. Alternatively, pharmaceutical aerosols for the delivery of antibodies to the lungs are, in one embodiment, introduced through direct delivery to a central airway. In another embodiment, pharmaceutical aerosols for the systemic delivery of antibodies to the lungs are inhaled via the nose, and not via the mouth. In another embodiment, pharmaceutical aerosols for the systemic delivery of antibodies to the lungs are inhaled via the mouth and via the nose.

The invention in one aspect provides a method for delivery of a therapeutic agent, wherein the method involves administering an effective amount of an aerosol of a conjugate of a therapeutic agent and an FcRn binding partner to lung such that a C/P ratio is at least 0.7.

A “therapeutic agent” as used herein refers to a compound useful to treat or prevent a disease, disorder, or condition of a subject. As used herein, the term “to treat” means to ameliorate the signs or symptoms of, or to stop the progression of, a disease, disorder, or condition of a subject. Signs, symptoms, and progression of a particular disease, disorder, or condition of a subject can be assessed using any applicable clinical or laboratory measure recognized by those of skill in the art, e.g., as described in Harrison 's Principles of Internal Medicine, 14^{th} Ed., Fauci A S et al., eds., McGraw-Hill, New York, 1998. As used herein, the term “subject” means a mammal; in one embodiment the subject is a human. For treating or preventing a particular disease, disorder, or condition, those of skill in the art will recognize a suitable therapeutic agent for that purpose.

The FcRn binding partner conjugates of the present invention can be utilized for the systemic delivery of a wide variety of therapeutic agents, including but not limited to, antigens, including tumor antigens; chemotherapy agents for the treatment of cancer; cytokines; growth factors; nucleic acid molecules and oligonucleotides, including DNA and RNA; hormones; fertility drugs; calcitonin, calcitriol and other bioactive steroids; antibiotics, including antibacterial agents, antiviral agents, antifungal agents, and antiparasitic agents; cell proliferation-stimulating agents; lipids; proteins and polypeptides; glycoproteins; carbohydrates; and any combination thereof. Specific examples of therapeutic agents are presented elsewhere herein. The FcRn binding partners of the present invention can further be utilized for the targeted delivery of a delivery vehicle, such as microparticles and liposomes.

As described in further detail below, a “conjugate” as used herein refers to two or more entities bound to one another by any physicochemical means, including, but not limited to, covalent interaction, hydrophobic interaction, hydrogen bond interaction, or ionic interaction. It is important to note that the bond between the FcRn binding partner and the therapeutic agent must be of such a nature and location that it does not destroy the ability of the FcRn binding partner to bind to the FcRn. Such bonds are well known to those of ordinary skill in the art, and examples are provided in greater detail below. The conjugate further can be formed as a fusion protein, also discussed in greater detail below.

The conjugate can include an intermediate or linker entity between the therapeutic agent and the FcRn binding partner, such that the therapeutic agent and the FcRn binding partner are bound to one another indirectly. In some embodiments the linker is subject to spontaneous cleavage. In some embodiments the linker is subject to assisted cleavage by an agent such as an enzyme or chemical. For example, protease-cleavable peptide linkers are well known in the art and include, without limitation, trypsin-sensitive sequence; plasmin-sensitive sequence; FLAG peptide; chymosin-sensitive sequence of bovine κ-casein A (Walsh M K et al. (1996) J Biotechnol 45:235-41); cathepsin B cleavable linker (Walker M A et al. (2002) Bioorg Med Chem Lett 12:217-9); thermolysin-sensitive poly(ethylene glycol) (PEG)-L-alanyl-L-valine (Ala-Val) (Suzawa T et al. (2000) J Control Release 69:27-41); enterokinase-cleavable linker (McKee C et al. (1998) Nat Biotechnol 16:647-51). Protease-cleavable peptide linkers can be designed for use and used in association with other major classes of proteases, e.g., matrix metalloproteinases and secretases (sheddases). Birkedal-Hansen H et al. (1993) Crit Rev Oral Biol Med 4:197-250; Hooper N M et al. (1997) Biochem J 321(Pt 2):265-79. In other embodiments the linker can be resistant to spontaneous, proteolytic, or chemical cleavage. An example of this type of linker is arginine-lysine-free linker (resistant to trypsin). Additional examples of linkers include, without limitation, polyglycine, (Gly)ₙ; polyalanine, (Ala)ₙ; poly(Gly-Ala), (Glyₘ-Ala)ₙ; poly (Gly-Ser), (e.g., Glyₘ-Ser)ₙ, and combinations thereof, where m and n are each independently an integer between 1 and 6. See also Robinson C R et al. (1998) Proc Natl Acad Sci USA 95:5929-34.

An “FcRn binding partner” as used herein refers to any entity that can be specifically bound by the FcRn and actively transported by the FcRn. FcRn binding partners of the present invention thus encompass, for example, whole IgG, the Fc fragment of IgG (i.e., Fcγ), other fragments of IgG that include the complete binding region for the FcRn, and other molecules that mimic FcRn-binding portions of Fcγ and bind to FcRn.

In certain embodiments the FcRn binding partner excludes FcRn-specific whole antibodies (i.e., anti-FcRn antibodies) which specifically bind FcRn through antigen-specific antigen-antibody interaction. It is to be understood in this context that antigen-specific antigen-antibody interaction means antigen binding specified by at least one complementarity determining region (CDR) within a hypervariable region of an antibody, e.g., a CDR within Fab, F(ab′), F(ab′)₂, and Fv fragments. Likewise, in certain embodiments the FcRn binding partner excludes FcRn-specific fragments, and analogs of FcRn-specific fragments, of whole antibodies which specifically bind FcRn through antigen-specific antigen-antibody interaction. Some such embodiments thus exclude FcRn-specific Fv fragments, single chain Fv (scFv) fragments, and the like. Other such embodiments exclude FcRn-specific Fab fragments, F(ab′) fragments, F(ab′)₂ fragments, and the like.

An important feature of this and all other aspects of the invention relates to the purposeful administration of the aerosolized conjugate or antibody to central airways of the lung. As explained in greater detail below, a “C/P ratio” is a measure of relative distribution of deposition of aerosolized particles to central airways of the lung in comparison to deposition to the periphery of the lung.

By way of further introduction to the central airway delivery feature of the invention, it is generally believed that the mechanisms of deposition of aerosol particles within airways include inertial impaction, interception, sedimentation, and diffusion. Inertial impaction occurs when large (high-mobility) particles or droplets travel in their initial direction of motion and do not follow the velocity streamlines as the direction of motion of the air passes around obstructions. These large particles travel to the obstruction and are deposited. Inertial impaction occurs throughout the tracheobronchial tree but particularly in the largest airways, where flow velocity and particle size are much larger. Interception is relevant in nasal deposition and in small airways. Particles will be intercepted when they enter an airstream moving in a direction of flow located less than the particles' diameter from the airway wall. Sedimentation takes place under the force of gravity and affects particles that are relatively large and are located in smaller airways of the alveolar region. Diffusion is responsible for the deposition of small, submicrometer particles. Particles move randomly under the influence of impact by gas molecules until they travel to the wall of the airway.

A number of factors contribute to the site of particle deposition within the lung, including the mechanics of breathing. Generally, the faster, shallower, and shorter the duration of inspiration, the more favorable for deposition in the central airways. Conversely, the slower, deeper, and longer the duration of inspiration, the more favorable for deposition in the periphery of the lung. Thus for example normal (i.e., tidal) breathing favors deposition in the central airways, whereas deep, supranormal inspiration and breath-holding favor deposition in the deep lung. Put another way, low flow, low pressure respiration favors deposition in the central airways, and conversely high flow, high pressure respiration favors deposition in the deep lung. Accordingly, in the setting of respiration on a mechanical ventilator, flow and pressure parameters controlled by the mechanical ventilator can be set to favor either central or peripheral deposition in the lungs. Such parameters for mechanically controlled or assisted breathing are selected on the basis of a number of clinical factors well known in the art, including body weight, underlying pulmonary or other disease, fraction of inspired oxygen (FiO₂), fluid volume status, lung compliance, etc., as well as the effective gas exchange as reflected by, e.g., blood pH, partial pressure of oxygen in the blood, and partial pressure of carbon dioxide in the blood.

Another factor affecting the site and extent of particle deposition within the airways relates to physicochemical characteristics of the particles. Important physicochemical characteristics of the particles include their aerodynamic diameter, mass density, velocity, and electrical charge. Some of these factors are considered in the following aspect of the invention.

Particle sizes in the range 2 μm to 10 μm are widely considered to be optimal for the delivery of therapeutic agents to the tracheobronchial and pulmonary regions. Heyder J et al. (1986) J Aerosol Sci 17:811-25. Maximal alveolar deposition has been shown to occur when particles have diameters between 1.5 μm and 2.5 μm and between 2.5 μm and 4 μm, with and without breath-holding techniques, respectively. Byron P R (1986) J Pharm Sci 75:433-38. As particle sizes increase beyond about 3 μm, deposition decreases in the alveoli and increases in the central airways. Beyond about 10 μm, deposition occurs predominantly in the larynx and upper airways.

Particle size and distribution are believed to be important parameters influencing aerosol deposition. Aerosol particles generally range in shape and size. The individual particle sizes of an aerosol may be characterized microscopically and an average primary particle size value can then be estimated, which describes the central tendency of the entire size distribution. It is convenient to express the particle size of irregularly shaped particles by an equivalent spherical dimension. The aerodynamic diameter (Dₐₑ) is defined as the diameter of a unit density sphere having the same settling velocity (generally in air) as the particle being studied. This dimension encompasses the particle's shape, density and physical size.

A population of particles can be defined in terms of the mass carried in each particle size range. This distribution can be divided into two equal halves at the mass median aerodynamic diameter (MMAD). The distribution around the MMAD can be expressed in terms of the geometric standard deviation (GSD). These parameters can be used if it is assumed that aerosol particle size distributions are log-normal.

Particle size, i.e., MMAD and GSD, can be measured using any suitable technique. Techniques widely employed include single- and multi-stage inertial impaction, virtual impaction, laser particle sizing, optical microscopy, and scanning electron microscopy. For a review, see Lalor C B et al. (1997) In: Inhalation Delivery of Therapeutic Peptides and Proteins, Adjei A L and Gupta P K, eds., New York: Marcel Dekker, pp 235-276.

Particles having a MMAD of at least 4.8 μm are non-respirable, i.e., they are believed not to enter the alveolar space in the deep lung. This explains why, prior to now, it has generally been preferred to administer aerosols characterized by particles having a MMAD of less than 5 μm. By contrast, in certain embodiments of the instant invention, a majority of the particles are non-respirable. In various embodiments a majority refers to at least 60 percent, at least 70 percent, at least 80 percent, at least 90 percent, and at least 95 percent.

Specialized aerosol generators are known to be capable of creating “monodisperse” aerosols, i.e., aerosols with particles having a GSD of less than 1.2 μm. Fuchs N A et al. (1966) In: Davies C N, ed., Aerosol Science, London: Academic Press, pp. 1-30. The vibrating orifice monodisperse aerosol generator (VOAG) is an example of one type of monodisperse aerosol generator, and it is frequently employed to prepare calibration standards. Berglund R N et al. (1973) Environ Sci Technol 7:147. This generator can achieve GSDs approaching 1.05 when concentrate is fed through the orifice plate having orifice diameters that range in size from 5 to 50 μm. Additional types of monodisperse aerosol generators include spinning disk and spinning top aerosol generators. These too are frequently employed to prepare calibration standards.

Those of skill in the art typically refer to a peripheral lung zone/central lung zone deposition ratio (P/C ratio) or, equivalently, the penetration index, as a measure of effective administration of agents to the deep lung. As the term suggests, the P/C ratio is a measure of relative distribution of deposition of aerosolized particles to the periphery of the lung in comparison to deposition to the central airways of the lung; it is thus the arithmetic inverse of the C/P ratio. The P/C ratio varies directly with the result that has until now typically been sought in order to achieve systemic delivery of the inhaled agent, i.e., administration directed to the deep lung. Typical P/C ratios sought for conventional applications are in the range of about 1.35 to 2.2 and higher. These typical P/C ratios correspond to C/P ratios of about 0.74 to 0.45 and lower.

Unlike these more typical applications, which call for maximizing administration to the periphery of the lung and thus a high P/C ratio, in the instant invention it is desirable to focus administration to the central airways of the lung. Thus in the instant invention it is desirable to achieve a relatively low P/C ratio, i.e., a high C/P ratio, in accordance with the surprising discovery that administration of FcRn binding partners to the central airways is advantageous when compared to administration to the periphery of the lung. Accordingly, the C/P ratio varies directly with the result that is sought in the instant invention, i.e., intended administration to the central airways of the lung. Accordingly, some embodiments include those for which the C/P ratio is at least 0.7. These embodiments specifically include those having C/P ratios of at least 0.7, 0.8, and 0.9. Additional embodiments include those for which the C/P ratio is at least 1.0-1.4. These embodiments specifically include those having C/P ratios of at least 1.0, 1.1, 1.2, 1.3, and 1.4. Yet other embodiments include those for which the C/P ratio is at least 1.5-1.9. These embodiments specifically include those having C/P ratios of at least 1.5, 1.6, 1.7, 1.8, and 1.9. Further embodiments include those for which the C/P ratio is at least: 2.0-3.0. These embodiments specifically include those having C/P ratios of at least 2.0, 2.1, 2.2, 2.3. 2.4, 2.5, 2.6, 2.7, 2.8, 2.9, and 3.0. There is no theoretical upper limit of the C/P ratio. Thus some embodiments include those having C/P ratios greater than 3.0.

Achievement of a C/P ratio of at least 0.7 is therefore favored by use of a normal or tidal breathing pattern as part of the method of administration. This can be accomplished, for example, by inhaling an aerosol over the course of a number of breaths during tidal breathing. In the setting of respiration on a mechanical ventilator, achievement of a C/P ratio of at least 0.7 is therefore favored by low flow, low pressure assisted ventilation as part of the method of administration.

Determination of the C/P ratio can be accomplished by any suitable method, but typically such determination involves planar imaging gamma scintigraphy, three-dimensional single-photon emission computed tomography (SPECT), or positron emission tomography (PET). Newman S P et al. (1998) Respiratory Drug Delivery VI:9-15; Fleming J S et al. (2000) J Aerosol Med 13:187-98. In a typical determination of the P/C ratio, an appropriate gamma ray emitting radionuclide, e.g., ^{99m}Tc, ^{113m}In, ¹³¹I, or ^{81m}Kr, is added to the drug formulation. After aerosol administration to a subject, data is acquired with a gamma camera and analysed by dividing the resulting lung images into two (central and peripheral) or three (central, intermediate, and peripheral) imaging regions. Newman S P et al., supra; Agnew J E et al. (1986) Thorax 41:524-30. Depending on the selected imaging method, the central imaging region or the central and intermediate imaging regions together are representative of central airways. The peripheral imaging region is representative of the periphery of the lung. Taking attenuation and decay into account, counts from the peripheral imaging region are divided by counts from the central imaging region (or, where appropriate, by combined counts from the central and intermediate imaging regions). Determination of the C/P ratio follows the method just outlined, but the ratio is calculated as counts from the central imaging region (or, where appropriate, combined counts from the central and intermediate imaging regions), divided by counts from the peripheral zone.

According to another aspect of the invention, a method is provided for systemic delivery of a therapeutic agent. The method according to this aspect involves administering an effective amount of an aerosol of a conjugate of a therapeutic agent and an FcRn binding partner to lung, wherein particles in the aerosol have a mass median aerodynamic diameter (MMAD) of at least 3 μm.

According to yet another aspect, the invention provides an aerosol of a conjugate of a therapeutic agent and an FcRn binding partner, wherein particles in the aerosol have a MMAD of at least 3 μm.

Because particle size may not be homogeneous, in various embodiments the particles having a Dₐₑ of at least 3 μm may constitute at least 50 percent, at least 60 percent, at least 70 percent, at least 75 percent, at least 80 percent, at least 85 percent, at least 90 percent, or at least 95 percent of the particles in the aerosol.

As mentioned previously, particles having a MMAD of at least 4.8 μm are non-respirable, i.e., they are believed not to enter the alveolar space in the deep lung. This explains why, prior to now, it has generally been preferred to administer aerosols characterized by particles having a MMAD of less than 5 μm. By contrast, in certain embodiments of the instant invention, a majority of the particles are non-respirable.

In yet another aspect the invention provides an aerosol delivery system. The aerosol delivery system according to this aspect includes a container, an aerosol generator connected to the container, and a conjugate of a therapeutic agent and an FcRn binding partner disposed within the container, wherein the aerosol generator is constructed and arranged to generate an aerosol of the conjugate having particles with a MMAD of at least 3 μm. As used herein, “connected” can in various embodiments refer to a direct connection or an indirect connection.

In one embodiment the aerosol delivery system includes a vibrational element constructed and arranged to vibrate an aperture plate having a plurality of apertures of defined geometry, wherein one side or surface of the aperture plate is in fluid connection with a solution or suspension of the conjugate. See, e.g., U.S. Pat. No. 5,758,637, U.S. Pat. No. 5,938,117, U.S. Pat. No. 6,014,970, U.S. Pat. No. 6,085,740, and U.S. Pat. No. 6,205,999, the entire contents of which are incorporated herein by reference. Activation of the vibrational element to vibrate the aperture plate causes liquid containing the conjugate in solution or suspension to be drawn through the plurality of apertures to create a low-velocity aerosol with a defined range of droplet (i.e., particle) sizes.

Examples of this type of aerosol generator are commercially available from Aerogen, Inc., Sunnyvale, Calif.

In another embodiment the aerosol delivery system includes a pressurized container containing the conjugate in solution or suspension. The pressurized container typically has an actuator connected to a metering valve so that activation of the actuator causes a predetermined amount of the conjugate in solution or suspension within the container to be dispensed from the container in the form of an aerosol. Pressurized containers of this type are well known in the art as propellant-driven metered-dose inhalers (pMDIs or simply MDIs). MDIs typically include an actuator, a metering valve, and a pressurized container that holds a micronized drug suspension or solution, liquefied propellant, and surfactant (e.g., oleic acid, sorbitan trioleate, lecithin). Historically these MDIs typically used chlorofluorocarbons (CFCs) as propellants, including trichlorofluoromethane, dichlorodifluoromethane, and dichlorotetrafluoromethane. Cosolvents such as ethanol may be present when the propellant alone is a relatively poor solvent. Newer propellants may include 1,1,1,2-tetrafluoroethane and 1,1,1,2,3,3,3-heptafluoropropane. Actuation of MDIs typically causes dose amounts of 50 μg-5 mg of active agent in volumes of 20-100 μL to be delivered at high velocity (30 m/sec) over 100-200 msec.

In other embodiments the aerosol delivery system includes an air-jet nebulizer or ultrasonic nebulizer in fluid connection with a reservoir containing the conjugate in solution or suspension. Nebulizers (air-jet or ultrasonic) are used primarily for acute care of nonambulatory patients and in infants and children. Air-jet nebulizers for atomization are considered portable because of the availability of small compressed air pumps, but they are relatively large and inconvenient systems. Ultrasonic nebulizers have the advantage of being more portable because they generally do not require a source of compressed air. Nebulizers provide very small droplets and high mass output. Doses administered by nebulization are much larger than doses in MDIs and the liquid reservoir is limited in size, resulting in short, single-duration therapy.

To generate an aerosol from an air-jet nebulizer, compressed air is forced through an orifice over the open end of a capillary tube, creating a region of low pressure. The liquid formulation is drawn through the tube to mix with the air jet and form the droplets. Baffles within the nebulizer remove larger droplets. The droplet size in the airstream is influenced by the compressed air pressure. Mass median diameters normally range from 2 to 5 μm with air pressures of 20 to 30 psig. The various commercially available air-jet nebulizers do not perform equally. This will affect the clinical efficacy of nebulized aerosol, which depends on the droplet size, total output from the nebulizer, and patient determinants.

Ultrasonic nebulizers generate aerosols using high-frequency ultrasonic waves (i.e., 100 kHz and higher) focused in the liquid chamber by a ceramic piezoelectric crystal that mechanically vibrates upon stimulation. Dennis J H et al. (1992) J Med Eng Tech 16:63-68; O'Doherty M J et al. (1992) Am Rev Respir Dis 146:383-88. In some instances, an impeller blows the particles out of the nebulizer or the aerosol is inhaled directly by the patient. The ultrasonic nebulizer is capable of greater output than the air-jet nebulizer and for this reason is used frequently in aerosol drug therapy. The droplets formed using ultrasonic nebulizers, which depend upon the frequency, are coarser (i.e., higher MMAD) than those delivered by air-jet nebulizers. The energy introduced into the liquid can result in an increase in temperature, which results in vaporization and variations in concentrations over time. This concentration variation over time is also encountered in jet nebulizers but is due to water loss through evaporation.

The choice between solution or suspension formulations in nebulizers is similar to that for the MDI. The formulation chosen will affect total mass output and particle size. Nebulizer formulations typically contain water with cosolvents (ethanol, glycerin, propylene glycol) and surfactants added to improve solubility and stability. Commonly an osmotic agent is also added to prevent bronchoconstriction from hypoosmotic or hyperosmotic solutions. Witeck T J et al. (1984) Chest 86:592-94; Desager K N et al. (1990) Agents Actions 31:225-28.

In yet other embodiments the aerosol delivery system includes a dry powder inhaler in fluid connection with a reservoir containing the conjugate in powder form. The dry powder inhaler device may eventually replace MDIs for some indications in response to the international control of chlorofluorocarbons in these latter products. Notably, this device can only deliver a fraction of its load in a respirable size range. Powder inhalers will usually disperse only about 10 to 20% of the contained drug into respirable particles. The typical dry powder inhaler device consists of two elements: the inhalation appliance to disperse unit doses of the powder formulation into the inspired airstream, and a reservoir of the powder formulation to dispense these doses. The reservoir typically can be of two different types. A bulk reservoir allows a precise quantity of powder to be dispensed upon individual dose delivery up to approximately 200 doses. A unit dose reservoir provides individual doses (e.g., provided in blister packaging or in gelatin capsule form) for inhalation as required. The hand-held device is designed to be manipulated to break open the capsule/blister package or to load bulk powder followed by dispersion from the patient's inspiration. Airflow will deaggregate and aerosolize the powder. In most cases, the patient's inspiratory airflow activates the device, provides the energy to disperse and deagglomerate the dry powder, and determines the amount of medicament that will reach the lungs.

Dry powder generators are subject to variability because of the physical and chemical properties of the powder. These inhalers are designed to meter doses ranging from 200 μg to 20 mg. The preparation of drug powder in these devices is very important. The powder in these inhalers requires efficient size reduction that is also needed for suspensions in MDIs. Micronized particles flow and are dispersed more unevenly than coarse particles. Therefore the micronized drug powder may be mixed with an inert carrier. This carrier is usually α-lactose monohydrate, because lactose comes in a variety of particle size ranges and is well characterized. Byron P R et al. (1990) Pharm Res 7(suppl):S81. The carrier particles have a larger particle size than the therapeutic agent to prevent the excipient from entering the airways. Segregation of the two particles will occur when turbulent airflow is created upon patient inhalation through the mouthpiece. This turbulence of inspiration will provide a certain amount of energy to overcome the interparticulate cohesive and particle surface adhesive forces for the micronized particles to become airborne. High concentrations of drug particles in air are easily attained using dry powder generation, but stability of the output and the presence of agglomerated and charged particles are common problems. With very small particles, dispersion is difficult because of electrostatic, van der Waals, capillary, and mechanical forces that increase their energy of association.

An example of a dry powder inhaler aerosol generator suitable for use with the present invention is the Spinhaler powder inhaler available from Fisons Corp., Bedford, Mass.

The FcRn molecule now is well characterized. As mentioned above, the FcRn has been isolated for several mammalian species, including humans. The FcRn occurs as a heterodimer involving an FcRn alpha chain (equivalently, FcRn heavy chain) and β₂ microglobulin. The sequence of the human FcRn, rat FcRn, and mouse FcRn alpha chains can be found in Story C M et al. (1994) J Exp Med 180:2377-81, which is incorporated herein by reference in its entirety. As will be recognized by those of ordinary skill in the art, FcRn can be isolated by cloning or by affinity purification using, for example, nonspecific antibodies, polyclonal antibodies, or monoclonal antibodies. Such isolated FcRn then can be used to identify and isolate FcRn binding partners, as described below.

The region of the Fc portion of IgG that binds to the FcRn has been described based upon X-ray crystallography (see, e.g., Burmeister W P et al (1994) Nature 372:379-83, and Martin W L et al. (2001) Mol Cell 7:867-77, which are incorporated by reference herein in their entirety). The major contact area of Fc with the FcRn is near the junction of the C_{H}2 and C_{H}3 domains. Potential IgG contacts are residues 248, 250-257, 272, 285, 288, 290-291, 307, 308-311 and 314 in C_{H}2 and 385-387,428 and 433-436 in C_{H}3. These sites are distinct from those identified by subclass comparison or by site-directed mutagenesis as important for Fc binding to leukocyte FcγRI and FcγRII. Previous studies have implicated murine IgG residues 253, 272, 285, 310, 311, and 433-436 as potential contacts with FcRn. Shields R L et al. (2001) J Biol Chem 276:6591-6604. In the human IgG1, a previous study has implicated residues 253-256, 288, 307,311, 312,380, 382, and 433-436 as potential contacts with FcRn. Shields R L et al. (2001) J Biol Chem 276:6591-6604. The foregoing Fc-FcRn contacts are all within a single Ig heavy chain. It has been noted previously that two FcRn can bind a single Fc homodimer. The crystallographic data suggest that in such a complex, each FcRn molecule has major contacts with one polypeptide of the Fc homodimer. Martin W L et al. (1999) Biochemistry 39:9698-708.

Human FcRn binds to all subclasses of human IgG but not as well to most subclasses of mouse and rat IgG. West A P et al. (2000) Biochemistry 39:9698-9708; Ober R J et al. (2001) Int Immunol 13:1551-59. Thus in certain embodiments the species of the subject to be treated corresponds to the species of origin of IgG from which FcRn binding partners can be derived. The order of affinities of binding within each species is IgG1=IgG2>IgG3>IgG4 (human); IgG1>IgG2b>IgG2a>IgG3 (mouse); and IgG2a>IgG1>IgG2b=IgG2c (rat). Burmeister W P et al (1994) Nature 372:379-83. It is believed, therefore, that human IgG (and FcRn contact-containing fragments thereof) belonging to any subclass is useful as a human FcRn binding partner.

In an embodiment of the present invention, FcRn binding partners other than whole IgG can be used to transport therapeutics across the pulmonary epithelial barrier. In such an embodiment, an FcRn binding partner can be chosen which binds the FcRn with higher affinity than whole lgG. Such an FcRn binding partner has utility in utilizing the FcRn to achieve active transport of a conjugated therapeutic across the epithelial barrier, and in reducing competition for the transport mechanism by endogenous IgG. The FcRn-binding activity of these higher affinity FcRn binding partners can be measured using standard assays known to those skilled in the art, including: (a) transport assays using polarized cells that naturally express the FcRn, or have been genetically engineered to express the FcRn or the alpha chain of the FcRn; (b) FcRn ligand:protein binding assays using soluble FcRn or fragments thereof, or immobilized FcRn; (c) binding assays utilizing polarized or non-polarized cells that naturally express the FcRn, or have been genetically engineered to express the FcRn or the alpha chain of the FcRn.

The FcRn binding partner can be produced by recombinant genetic engineering techniques. Within the scope of the invention are nucleotide sequences encoding human FcRn binding partners. The FcRn binding partners include whole IgG, the Fc fragment of IgG and other fragments of IgG that include the complete binding region for the FcRn. The major contact sites include amino acid residues 248, 250-257, 272, 285, 288, 290-291, 308-311 and 314 of the C_{H}2 domain and amino acid residues 385-387, 428 and 433-436 of the C_{H}3 domain. Therefore in one embodiment of the present invention are nucleotide sequences encoding regions of the IgG Fc fragment spanning these amino acid residues.

The Fc region of IgG can be modified according to well recognized procedures such as site-directed mutagenesis and the like to yield modified IgG or modified Fc fragments or portions thereof that will be bound by the FcRn. Such modifications include modifications remote from the FcRn contact sites as well as modifications within the contact sites that preserve or even enhance binding to the FcRn. For example, the following single amino acid residues in human IgG1 Fc (Fcγ1) can be substituted without significant loss of Fc binding affinity for FcRn: P238A, S239A, K246A, K248A, D249A, M252A, T256A, E258A, T260A, D265A, S267A, H268A, E269A, D270A, E272A, L274A, N276A, Y278A, D280A, V282A, E283A, H285A, N286A, T289A, K290A, R292A, E293A, E294A, Q295A, Y296F, N297A, S298A, Y300F, R301A, V303A, V305A, T307A, L309A, Q311A, D312A, N315A, K317A, E318A, K320A, K322A, S324A, K326A, A327Q, P329A, A330Q, P331A, E333A, K334A, T335A, S337A, K338A, K340A, Q342A, R344A, E345A, Q347A, R355A, E356A, M358A, T359A, K360A, K360A, N361A, Q362A, Y373A, S375A, D376A, A378Q, E380A, E382A, S383A, N384A, Q386A, E388A, N389A, N390A, Y391F, K392A, L398A, S400A, D401A, D413A, K414A, R416A, Q418A, Q419A, N421A, V422A, S424A, E430A, N434A, T437A, Q438A, K439A, S440A, S444A, and K447A, where for example P238A represents wildtype proline at position 238 substituted by alanine. Shields R L et al. (2001) J Biol Chem 276:6591-6604. Many but not all of the variants listed above are alanine variants, i.e., the wildtype residue is replaced by alanine. In addition to alanine, however, other amino acids can be substituted for the wildtype amino acids at the positions specified above. These mutations can be introduced singly into Fc, giving rise to more than one hundred FcRn binding partners structurally distinct from native human Fcγ1. Furthermore, combinations of two, three, or more of these individual mutations can be introduced together, giving rise to yet additional FcRn binding partners.

Certain of the above mutations can confer new functionality upon the FcRn binding partner. For example, one embodiment incorporates N297A, removing a highly conserved N-glycosylation site. The effect of this mutation is to reduce immunogenicity, thereby enhancing circulating half-life of the FcRn binding partner, and to render the FcRn binding partner essentially incapable of binding to FcγRI, FcγRIIA, FcγRIIB, and FcγRIIIA, without compromise of its affinity for FcRn. Routledge E G et al. (1995) Transplantation 60:847-53; Friend P J et al. (1999) Transplantation 68:1632-37;Shields R L et al. (2001) J Biol Chem 276:6591-6604.

As a further example of new functionality arising from mutations above, affinity for FcRn can be increased beyond that of wildtype in some instances. This increased affinity can reflect an increased “on” rate, a decreased “off” rate, or both an increased “on” rate and a decreased “off” rate. Mutations believed may impart an increased affinity for FcRn include in particular T256A, T307A, E380A, and N434A. Shields R L et al. (2001) J Biol Chem 276:6591-6604. Combination variants believed may impart an increased affinity for FcRn include in particular E380A/N434A, T307A/E380A/N434A, and K288A/N434A. Shields R L et al. (2001) J Biol Chem 276:6591-6604.

In addition to the FcRn binding partners disclosed above, in one embodiment, the FcRn binding partner is a polypeptide including the sequence: PKNSSMISNTP (SEQ ID NO: 11), and optionally further including a sequence chosen from HQSLGTQ (SEQ ID NO: 12), HQNLSDGK (SEQ ID NO: 13), HQNISDGK (SEQ ID NO: 14), or VISSHLGQ (SEQ ID NO: 15). U.S. Pat. No. 5,739,277 issued to Presta et al. The sequence PKNSSMISNTP (SEQ ID NO: 11) is to be compared with the sequence PKDTLMISRTP (SEQ ID NO: 16) corresponding to amino acids 247-257 in the C_{H}2 domain of Fc (SEQ ID NO: 2). The latter sequence encompasses nine amino acids previously noted to be believed to be major contact sites with FcRn.

It is not intended that the invention be limited by the selection of any particular FcRn binding partner. Thus, in addition to the FcRn binding partners just described, other binding partners can be identified and isolated. Antibodies or portions thereof specific for the FcRn and capable of being transported by FcRn once bound can be identified and isolated using well established techniques. Likewise, randomly generated molecularly diverse libraries can be screened and molecules that are bound and transported by FcRn can be isolated using conventional techniques. FcRn binding partners incorporating modifications to the polypeptide (i.e., polyamide) backbone, as distinguished from substitutions of the amino acid side chain groups, are also contemplated by the invention. For example, Bartlett et al. reported phosphonate-, phosphinate- and phosphinamide-containing pseudopeptide inhibitors of pepsin and penicillopepsin. Bartlett et al. (1990) J Org Chem 55:6268-74. See also U.S. Pat. No. 5,563,121. Those inhibitors were pseudopeptides that included a phosphorus-containing bond in place of the scissile amide bond that would normally be cleaved by those enzymes.

In vitro screening methods for identifying and characterizing FcRn binding partners may be based on techniques familiar to those of skill in the art. These may include enzyme-linked immunosorbent assay (ELISA), where isolated FcRn is bound, directly or indirectly, to a substrate as a “capture antigen” and subsequently exposed to a sample containing a test FcRn binding partner; binding of the test FcRn binding partner to the immobilized FcRn is then assayed directly or indirectly. In related methods, competitive ELISA or direct radioimmunoassay (RIA) may be used to determine affinity of an unlabeled test FcRn binding partner for FcRn relative to the affinity of a labeled standard FcRn binding partner for FcRn. These techniques are readily scalable and therefore suitable for large-scale and high throughput screening of candidate FcRn binding partners.

Additional in vitro screening methods useful for identifying and characterizing FcRn binding partners can be cell-based. These methods measure cell binding, cell uptake, or cell transcytosis of the test FcRn binding partner. Such methods may be facilitated by labeling the FcRn binding partner with, for example, an isotope (¹³¹I, ³⁵S, ³²P, ¹³C, etc.), a chromophore, a fluorophore, biotin, or an epitope recognized by an antibody (e.g., FLAG peptide). The cells used in these assays may express FcRn either naturally or as a result of introduction into the cells of an isolated nucleic acid molecule encoding FcRn, operatively linked to a suitable regulatory sequence. Typically the nucleic acid encoding FcRn, operatively linked to a suitable regulatory sequence, is a plasmid that is used to transform or transfect a host cell. Methods for transient and stable transformation and transfection are well known in the art, and they include physical, chemical, and viral techniques, for example calcium phosphate precipitation, electroporation, biolistic injection, and others.

Yet other in vitro methods suitable for identifying and characterizing FcRn binding partners may include flow cytometry (FACS), electromobility shift assay (EMSA), surface plasmon resonance (biomolecular interaction analysis; BIAcore), chip-based surface interaction analysis, and others.

If the FcRn binding partner is a peptide composed entirely of gene-encoded amino acids, or a portion of it is so composed, the peptide or the relevant portion can also be synthesized using conventional recombinant genetic engineering techniques. For recombinant production, a polynucleotide sequence encoding the FcRn binding partner is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected or otherwise introduced into a suitable target cell which will express the peptide. Depending on the expression system used, the expressed peptide is then isolated by procedures well-established in the art. Methods for recombinant protein and peptide production and isolation are well known in the art (see, e.g., Maniatis et al., 1989, Molecular Cloning: A Laboratory Manual, Cold Spring Harbor Laboratory, N.Y.; and Ausubel et al., 1989, Current Protocols in Molecular Biology, Greene Publishing Associates and Wiley Interscience, New York). Indeed, isolation of Fc-containing molecules involves particularly well known affinity chromatography and related methods using protein A, protein G, or synthetic analogs thereof.

To increase efficiency of production, the polynucleotide can be designed to encode multiple units of the FcRn binding partner separated by enzymatic cleavage sites. The resulting polypeptide can be cleaved (e.g., by treatment with the appropriate enzyme) in order to recover the peptide units. This can increase the yield of peptides driven by a single promoter. When used in appropriate viral expression systems, the translation of each peptide encoded by the mRNA is directed internally in the transcript, e.g., by an internal ribosome entry site, IRES. Thus, the polycistronic construct directs the transcription of a single, large polycistronic mRNA which, in turn, directs the translation of multiple, individual peptides. This approach eliminates the production and enzymatic processing of polyproteins and can significantly increase yield of peptide driven by a single promoter.

A variety of host-expression vector systems can be utilized to express the FcRn binding partners described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus (CaMV) or tobacco mosaic virus (TMV)) or transformed with recombinant plasmid expression vectors (e.g., Ti plasmid) containing an appropriate coding sequence; or animal cell systems. Various host-expression systems are well known by those of skill in the art, and the host cell and expression vector elements are available from commercial sources.

The expression elements of the expression systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter; the cytomegalovirus (CMV) promoter) may be used; when generating cell lines that contain multiple copies of expression product, SV40-, BPV- and EBV-based vectors may be used with an appropriate selectable marker.

In cases where plant expression vectors are used, the expression of sequences encoding the polypeptides of the invention may be driven by any of a number of promoters. For example, viral promoters such as the 35S RNA and 19S RNA promoters of CaMV (Koziel M G et al. (1984) J Mol Appl Genet 2:549-62), or the coat protein promoter of TMV may be used; alternatively, plant promoters such as the small subunit of RUBISCO (Coruzzi G et al. (1984) EMBO J 3:1671-79; Broglie R et al. (1984) Science 224:838-43) or heat shock promoters, e.g., soybean hsp17.5-E or hsp17.3-B (Gurley W B et al. (1986) Mol Cell Biol 6:559-65) may be used. These constructs can be introduced into plant cells using Ti plasmids, Ri plasmids, plant virus vectors, direct DNA transformation, microinjection, electroporation, etc. For reviews of such techniques see, e.g., Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

In one insect expression system that may be used to express the FcRn binding partners, Autographa californica nuclear polyhidrosis virus (AcNPV) is used as a vector to express the foreign genes. The virus grows in Spodoptera frugiperda cells. A coding sequence may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., see U.S. Pat. No. 4,745,051). Further examples of this expression system may be found in Current Protocols in Molecular Biology, Vol. 2, Ausubel et al., eds., Greene Publishing Associates and Wiley Interscience, N.Y.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts (see, e.g., Logan J et al. (1984) Proc Natl Acad Sci USA 81:3655-59). Alternatively, the vaccinia 7.5 K promoter may be used, (see, e.g., Mackett M et al. (1982) Proc Natl Acad Sci USA 79:7415-19; Mackett M et al. (1984) J Virol 49:857-64; Panicali S et al. (1982) Proc Natl Acad Sci USA 79:4927-31).

Also for use in mammalian host cells are a number of eukaryotic expression plasmids. These plasmids typically include a promoter or promoter/enhancer element operably linked to the inserted gene or nucleic acid of interest, a polyadenylation signal positioned downstream of the inserted gene, a selection marker, and an origin of replication. Some of these plasmids are designed to accept nucleic acid inserts at specified positions, either as PCR products or as restriction enzyme digest products. Examples of eukaryotic expression plasmids include pRc/CMV, pcDNA3.1, pcDNA4, pcDNA6, pGene/V5 (Invitrogen), and pED.dC (Genetics Institute).

The FcRn binding partner is in some embodiments conjugated with an antigen. An antigen as used herein falls into four classes: (1) antigens that are characteristic of a pathogen; (2) antigens that are characteristic of an autoimmune disease; (3) antigens that are characteristic of an allergen; and (4) antigens that are characteristic of a cancer or tumor. Antigens in general include polysaccharides, glycolipids, glycoproteins, peptides, proteins, carbohydrates and lipids from cell surfaces, cytoplasm, nuclei, mitochondria and the like.

Antigens that are characteristic of pathogens include antigens derived from viruses, bacteria, parasites or fungi. Examples of important pathogens include Vibrio cholerae, enterotoxigenic Escherichia coli, rotavirus, Clostridium difficile, Shigella species, Salmonella typhi, parainfluenza virus, influenza virus, Streptococcus pneumoniae, Borrelia burgdorferi, HIV, Streplococcus mutans, Plasmodium falciparum, Staphylococcus aureus, rabies virus and Epstein-Barr virus.

Viruses in general include but are not limited to those in the following families: picornaviridae; caliciviridae; togaviridae; flaviviridae; coronaviridae; rhabdoviridae; filoviridae; paramyxoviridae; orthomyxoviridae; bunyaviridae; arenaviridae; reoviridae; retroviridae; hepadnaviridae; parvoviridae; papovaviridae; adenoviridae; herpesviridae; and poxviridae.

Bacteria in general include but are not limited to: Pseuclomonas spp., including P. aeruginosa and P. cepacia; Escherichia spp., including E. coli, E. faecalis; Klebsiella spp.; Serratia spp.; Acinetobacter spp.; Streptococcus spp., including S. pneumoniae, S. pyogenes, S. bovis, S. agalactiae; Staphylococcus spp., including S. aureus, S. epidermidis; Haemophilus spp.; Neisseria spp., including N. meningitidis; Bacteroides spp.; Citrobacter spp.; Branhamella spp.; Salmonella spp.; Shigella spp.; Proteus spp., including P. mirabilis; Clostridium spp.; Erysipelothrix spp.; Listeria spp.; Pasteurella multocida; Streptobacillus spp.; Spirillum spp.; Fusospirocheta spp.; Treponema pallidum; Borrelia spp.; Actinomycetes; Mycoplasma spp.; Chlamydia spp.; Rickettsia spp.; Spirochaeta; Legionella spp.; Mycobacteria spp., including M tuberculosis, M. kansasii, M. intracellulare, M marinum; Ureaplasma spp.; Streptomyces spp.; and Trichomonas spp.

Parasites include but are not limited to: Plasmodium falciparum, P. vivax, P. ovale, P. malaria; Toxoplasma gondii; Leishmania mexicana, L. tropica, L. major, L. aethiopica, L. donovani, Trypanosoma cruzi, T brucei, Schistosoma mansoni, S. haematobium, S. japonium; Trichinella spiralis; Wuchereria bancrofti; Brugia malayi; Entamoeba histolytica; Enterobius vermicularis; Taenia solium, T saginata, Trichomonas vaginalis, T hominis, T tenax; Giardia lamblia; Cryptosporidium parvum; Pneumocystis carinii, Babesia bovis, B. divergens, B. microti, Isospora belli, L. hominis; Dientamoebafragilis; Onchocerca volvulus; Ascaris lumbricoides; Necator americanis; Ancylostoma duodenale; Strongyloides stercoralis; Capillaria philippinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchisfelineas, G. viverini, Fasciola hepatica, Sarcoptes scabiei, Pediculus humanus; Phthirlus pubis; and Dermatobia hominis.

Fungi in general include but are not limited to: Cryptococcus neoformans; Blastomyces dermatitidis; A iellomyces dermatitidis; Histoplasma capsulatum; Coccidioides immitis; Candida species, including C. albicans, C. tropicalis, C parapsilosis, C. guilliermondii and C. krusei; Aspergillus species, including A. fumigatus, A. flavus and A. niger; Rhizopus species; Rhizomucor species; Cunninghammella species; Apophysomyces species, including A. saksenaea, A. mucor and A. absidia; Sporothrix schenckii; Paracoccidioides brasiliensis; Pseudallescheria boydii; Torulopsis glabrata; and Dermatophytes species.

Antigens that are characteristic of autoimmune disease typically will be derived from the cell surface, cytoplasm, nucleus, mitochondria and the like of mammalian tissues. Examples include antigens characteristic of uveitis (e.g., S antigen), diabetes mellitus, multiple sclerosis, systemic lupus erythematosus, Hashimoto's thyroiditis, myasthenia gravis, primary myxoedema, thyrotoxicosis, rheumatoid arthritis, pernicious anemia, Addison's disease, scleroderma, autoimmune atrophic gastritis, premature menopause, male infertility, juvenile diabetes, Goodpasture's syndrome, pemphigus vulgaris, pemphigoid, sympathetic ophthalmia, phacogenic uveitis, autoimmune haemolytic anemia, idiopathic thrombocytopenic purpura, idiopathic leukopenia, primary biliary cirrhosis, ulcerative colitis, Sjögren's syndrome, Wegener's granulomatosis, poly/dermatomyositis, and discoid lupus erythematosus. It is to be understood that an antigen characteristic of autoimmune disease refers to an antigen against which a subject's own immune system makes antibodies or specific T cells, and those antibodies or T cells are characteristic of an autoimmune disease. The specific identity of an antigen characteristic of an autoimmune disease in many cases is not, and indeed for the purposes of the invention need not, be known.

Antigens that are allergens are generally proteins or glycoproteins, although allergens may also be low molecular weight allergenic haptens that induce allergy after covalently combining with a protein carrier (Remington 's Pharmaceutical Sciences). Allergens include antigens derived from pollens, dust, molds, spores, dander, insects and foods. Specific examples include the urushiols (pentadecylcatechol or heptadecyicatechol) of Toxicodendron species such as poison ivy, poison oak and poison sumac, and the sesquiterpenoid lactones of ragweed and related plants.

Antigens that are characteristic of tumor antigens typically will be derived from the cell surface, cytoplasm, nucleus, organelles and the like of cells of tumor tissue. Examples include antigens characteristic of tumor proteins, including proteins encoded by mutated oncogenes; viral proteins associated with tumors; and tumor mucins and glycolipids. Tumors include, but are not limited to, those from the following sites of cancer and types of cancer: lip, nasopharynx, pharynx and oral cavity, esophagus, stomach, small intestine, colon, rectum, liver, gall bladder, biliary tree, pancreas, larynx, lung and bronchus, melanoma, breast, cervix, uterus, ovary, bladder, kidney, brain and other parts of the nervous system, thyroid, prostate, testes, bone, muscle, Hodgkin's disease, non-Hodgkin's lymphoma, multiple myeloma and leukemia. Viral proteins associated with tumors would be those from the classes of viruses noted above. An antigen characteristic of a tumor may be a protein not usually expressed by a tumor precursor cell, or may be a protein which is normally expressed in a tumor precursor cell, but having a mutation characteristic of a tumor. An antigen characteristic of a tumor may be a mutant variant of the normal protein having an altered activity or subcellular distribution. Mutations of genes giving rise to tumor antigens, in addition to those specified above, may be in the coding region, 5′ or 3′ noncoding regions, or introns of a gene, and may be the result of point mutations, frameshifts, inversions, deletions, additions, duplications, chromosomal rearrangements and the like. One of ordinary skill in the art is familiar with the broad variety of alterations to normal gene structure and expression which gives rise to tumor antigens.

Specific examples of tumor antigens include: proteins such as Ig-idiotype of B cell lymphoma; mutant cyclin-dependent kinase 4 of melanoma; Pmel-17 (gp 100) of melanoma; MART-1 (Melan-A) of melanoma (PCT publication WO94/21126); p15 protein of melanoma; tyrosinase of melanoma (PCT publication WO94/14459); MAGE 1, 2 and 3 of melanoma, thyroid medullary, small cell lung cancer, colon and/or bronchial squamous cell cancer (PCT/US92/04354); MAGE-Xp (U.S. Pat. No. 5,587,289); BAGE of bladder, melanoma, breast, and squamous-cell carcinoma (U.S. Pat. No. 5,571,711 and PCT publication WO95/00159); GAGE (U.S. Pat. No. 5,610,013 and PCT publication WO95/03422); RAGE family (U.S. Pat. No. 5,939,526); PRAME (formerly DAGE; PCT publication WO96/10577); MUM-1/LB-33B (U.S. Pat. No. 5,589,334); NAG (U.S. Pat. No. 5,821,122); FB5 (endosialin) (U.S. Pat. No. 6,217,868); PSMA (prostate-specific membrane antigen; U.S. Pat. No. 5,935,818); gp75 of melanoma; oncofetal antigen of melanoma; carbohydrate/lipids such as mucin of breast, pancreas, and ovarian cancer; GM2 and GD2 gangliosides of melanoma; oncogenes such as mutant p53 of carcinoma; mutant ras of colon cancer; HER2/neu proto-oncogene of breast carcinoma; and viral products such as human papillomavirus proteins of squamous cell cancers of cervix and esophagus. The foregoing list is only intended to be representative and is not to be understood to be limiting. It is also contemplated that proteinaceous tumor antigens may be presented by HLA molecules as specific peptides derived from the whole protein. Metabolic processing of proteins to yield antigenic peptides is well known in the art (see, e.g., U.S. Pat. No. 5,342,774, issued to Boon et al., which is incorporated herein by reference in its entirety). The present method thus encompasses delivery of antigenic peptides and such peptides in a larger polypeptide or whole protein which give rise to antigenic peptides. Delivery of antigenic peptides or proteins may give rise to humoral or cellular immunity.

Generally, subjects can receive an effective amount of an antigen, including a tumor antigen, and/or a peptide derived therefrom, by one or more of the methods detailed below. Initial doses can be followed by booster doses, following immunization protocols standard in the art. Delivery of antigens, including tumor antigens, thus may stimulate proliferation of cytolytic T lymphocytes.

In the cases of protein and peptide therapeutic agents, covalent linking to an FcRn binding partner is intended to include linkage by peptide bonds in a single polypeptide chain. Established methods (Sambrook et al., Molecular Cloning: A Laboratory Manual, Cold Spring Harbor Press, Cold Spring Harbor, N.Y. 1989, which is incorporated herein by reference in its entirety) would be used to engineer DNA encoding a fusion protein comprised of the protein or peptide therapeutic agent and an FcRn binding partner. This DNA would be placed in an expression vector and introduced into bacterial, eukaryotic, or other suitable host cells by established methods. The fusion protein would be purified from the cells or from the culture medium by established methods. The purification scheme may conveniently use isolated or recombinant protein A or protein G to purify FcRn binding partner-containing fusion proteins from host cell products. Such resulting conjugates include fusions of the FcRn binding partner to a protein, peptide or protein derivative such as those listed herein including, but not limited to, antigens, allergens, pathogens or to other proteins or protein derivatives of potential therapeutic interest such as growth factors, colony stimulating factors, growth inhibitory factors, signaling molecules, hormones, steroids, neurotransmitters, or morphogens that would be of use when delivered across an epithelial barrier.

By way of example, but not limitation, proteins used in fusion proteins to synthesize conjugates may include EPO (U.S. Pat. Nos. 4,703,008; 5,457,089; 5,614,184; 5,688,679; 5,773,569; 5,856,298; 5,888,774; 5,986,047; 6,048,971; 6,153,407), IFN-α (U.S. Pat. Nos. 4,678,751; 4,801,685; 4,820,638; 4,921,699; 4,973,479; 4,975,276; 5,098,703; 5,310,729; 5,869,293; 6,300,474), IFN-β (U.S. Pat. Nos. 4,820,638; 5,460,811), FSH (U.S. Pat. Nos. 4,923,805; 5,338,835; 5,639,639; 5,639,640; 5,767,251; 5,856,137), platelet-derived growth factor (PDGF; U.S. Pat. No. 4,766,073), platelet-derived endothelial cell growth factor (PD-ECGF; U.S. Pat. No. 5,227,302), human pituitary growth hormone (hGH; U.S. Pat. No. 3,853,833), TGF-β (U.S. Pat. No. 5,168,051), TGF-α (U.S. Pat. No. 5,633,147), keratinocyte growth factor (KGF; U.S. Pat. No. 5,731,170), insulin-like growth factor I (IGF-I; U.S. Pat. No. 4,963,665), epidermal growth factor (EGF; U.S. Pat. No. 5,096,825), granulocyte-macrophage colony-stimulating factor (GM-CSF; U.S. Pat. No. 5,200,327), macrophage colony-stimulating factor (M-CSF; U.S. Pat. No. 5,171,675), colony stimulating factor-1 (CSF-1; U.S. Pat. No. 4,847,201), Steel factor, Calcitonin, AP-1 proteins (U.S. Pat. No. 5,238,839), Factor VIIa, Factor VIII, Factor IX, TNF-α, TNF-α receptor, LFA-3, CNTF, CTLA-4, leptin (PCT/US95/10479, WO 96/05309), and brain-derived neurotrophic factor (BDNF; U.S. Pat. No. 5,229,500). All of the references cited above are incorporated herein by reference in their entirety.

By way of example, but not limitation, peptides used in fusion proteins to synthesize conjugates can include erythropoietin mimetic peptides (EPO receptor agonist peptides; PCT/US01/14310; WO 01/83525; Wrighton N C et al. (1996) Science 273:458-64; PCT/US99/05842, WO 99/47151), EPO receptor antagonist peptides (PCT/US99/05842, WO 99/47151; McConnell S J et al. (1998) Biol Chem 379:1279-86), and T20 (PCT/US00/35724; WO 01/37896).

In one embodiment, the fusion proteins of the invention are constructed and arranged so that the FcRn binding partner portion of the conjugate occurs downstream of the therapeutic agent portion, i.e., the FcRn binding partner portion is C-terminal with respect to the therapeutic agent portion. This arrangement is expressed in a short-hand manner as X-Fc, where “X” represents the therapeutic agent portion and Fc represents the FcRn binding partner portion. In this short-hand notation, “Fc” can be, but is not limited to, Fc fragment of IgG. The notation “X-Fc” is to be understood to encompass fusion proteins in which is present a linker joining the X and FcRn binding partner components.

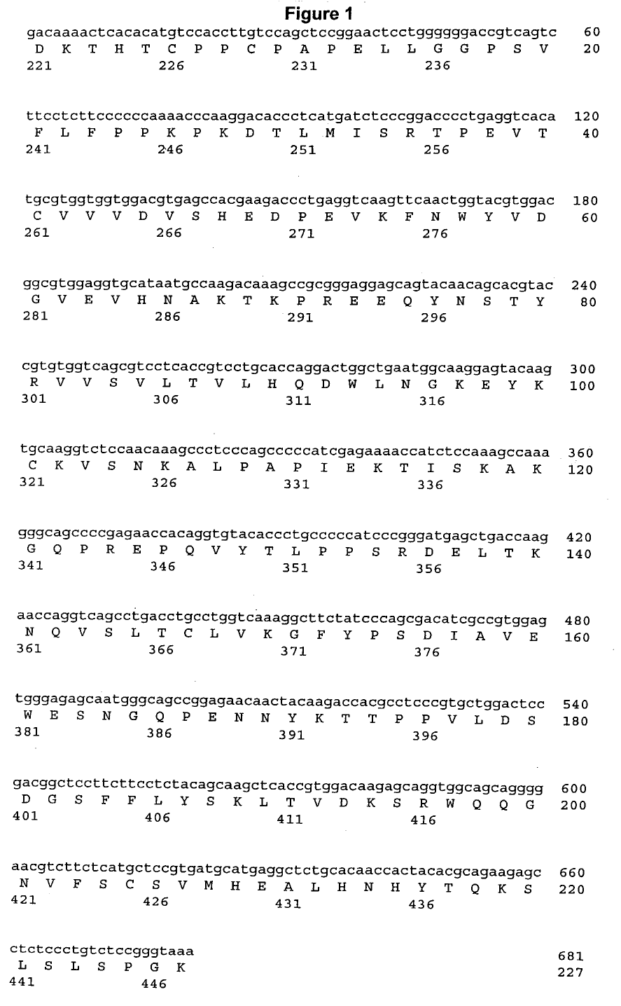

In one embodiment, fusion proteins of the present invention are constructed in which the conjugate consists of an Fc fragment of human IgG1 (starting with the amino acids D-K-T-H at the N-terminus of the hinge (see SEQ ID NO: 2, FIG. 1), including the hinge and C_{H}2 domain, and continuing through the S—P-G-K sequence in the C_{H}3 domain) fused to one of the polypeptide therapeutic agents listed herein. In one embodiment, a nucleotide sequence encoding functional EPO is fused in proper translational reading frame 5′ to a nucleotide sequence encoding the hinge, C_{H}2 domain, and C_{H}3 domain of the constant heavy (C_{H}) chain of human IgG1. This particular embodiment is described in more detail in Example 3.

Published European patent application EP 0 464 533 A discloses an EPO-FC fusion protein.

Published PCT application PCT/US00/19336 (WO 01/03737) discloses a human EPO-Fc fusion protein.

Published PCT application PCT/US98/13930 (WO 99/02709) discloses EPO-Fc and Fc-EPO fusion proteins.

Published PCT application PCT/EP00/10843 (WO 01/36489) discloses a number of Fc-EPO fusion proteins.

Published PCT application PCT/US00/19336 (WO 01/03737) discloses a human IFN-α-Fc fusion protein.

U.S. Pat. No. 5,723,125 issued to Chang et al. discloses a human IFN-α-Fc fusion protein wherein the IFN-α and Fc domains are connected through a particular Gly-Ser linker (Gly-Gly-Ser-Gly-Gly-Ser-Gly-Gly-Gly-Gly-Ser-Gly-Gly-Gly-Gly-Ser; SEQ ID NO: 17).

Published PCT application PCT/US00/13827 (WO 00/69913) discloses an Fc-IFN-α fusion protein.

Published PCT application PCT/US00/19336 (WO 01/03737) discloses a human IFN-β-Fc fusion protein.

Published PCT application PCT/US99/24200 (WO 00/23472) discloses a human IFN-β-Fc fusion protein.

U.S. Pat. No. 5,908,626 issued to Chang et al. discloses a human IFN-β-Fc fusion protein wherein the IFN-β and Fc domains are connected through a particular Gly-Ser linker (Gly-Gly-Ser-Gly-Gly-Ser-Gly-Gly-Gly-Gly-Ser-Gly-Gly-Gly-Gly-Ser; SEQ ID NO: 17).

U.S. Pat. No. 5,726,044 issued to Lo et al., and published PCT application PCT/US00/19816 (WO 01/07081), discloses an Fc-PSMA fusion construct.

The FcRn binding partners can be conjugated to a variety of therapeutic agents for targeted systemic delivery. The present invention encompasses the targeted systemic delivery of biologically active substances.

As used herein, the term “biologically active substance” refers to eukaryotic and prokaryotic cells, viruses, vectors, proteins, peptides, nucleic acids, polysaccharides and carbohydrates, lipids, glycoproteins, and combinations thereof, and naturally-occurring, synthetic, and semi-synthetic organic and inorganic drugs exerting a biological effect when administered to an animal. For ease of reference, the term is also used to include detectable compounds such as radio-opaque compounds including barium, as well as magnetic compounds. The biologically active substance can be soluble or insoluble in water. Examples of biologically active substances include anti-angiogenesis factors, antibodies, growth factors, hormones, enzymes, and drugs such as steroids, anti-cancer drugs and antibiotics.

In diagnostic embodiments, the FcRn binding partners may also be conjugated to a pharmaceutically acceptable gamma-emitting moiety, including but not limited to, indium and technetium, magnetic particles, radio-opaque materials such as barium, and fluorescent compounds.

By way of example, and without limitation, the following classes of drugs can be conjugated to FcRn binding partners for the purposes of systemic delivery across pulmonary epithelial barrier:

Antineoplastic Compounds. Nitrosoureas, e.g., carmustine, lomustine, semustine, strepzotocin; Methylhydrazines, e.g., procarbazine, dacarbazine; steroid hormones, e.g., glucocorticoids, estrogens, progestins, androgens, tetrahydrodesoxycaricosterone, cytokines and growth factors; Asparaginase.

Immunoactive Compounds. Immunosuppressives, e.g., pyrimethamine, trimethopterin, penicillamine, cyclosporine, azathioprine; immunostimulants, e.g., levamisole, diethyl dithiocarbamate, enkephalins, endorphins.

Antimicrobial Compounds. Antibiotics, e.g., penicillins, cephalosporins, carbapenims and monobactams, β-lactamase inhibitors, aminoglycosides, macrolides, tetracyclins, spectinomycin; Antimalarials; Amebicides; Antiprotazoal agents; Antifungal agents, e.g., amphotericin B; Antiviral agents, e.g., acyclovir, idoxuridine, ribavirin, trifluridine, vidarabine, gancyclovir.

Gastrointestinal Drugs. Histamine H₂ receptor antagonists, proton pump inhibitors, promotility agents.

Hematologic Compounds. Immunoglobulins; blood clotting proteins; e.g., antihemophiliac factor, factor IX complex; anticoagulants, e.g., dicumarol, heparin Na; fibrolysin inhibitors, tranexamic acid.

Cardiovascular Drugs. Peripheral antiadrenergic drugs, centrally acting antihypertensive drugs, e.g., methyldopa, methyldopa HCl; antihypertensive direct vasodilators, e.g., diazoxide, hydralazine HCl; drugs affecting renin-angiotensin system; peripheral vasodilators, phentolamine; antianginal drugs; cardiac glycosides; inodilators; e.g., amrinone, milrinone, enoximone, fenoximone, imazodan, sulmazole; antidysrhythmic; calcium entry blockers; drugs affecting blood lipids.

Neuromuscular Blocking Drugs. Depolarizing, e.g., atracurium besylate, hexafluorenium Br, metocurine iodide, succinylcholine Cl, tubocurarine Cl, vecuronium Br; centrally acting muscle relaxants, e.g., baclofen.

Neurotransmitters and Neurotransmitter Agents. Acetylcholine, adenosine, adenosine triphosphate, amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K+ channel toxins.

Antiparkinson Drugs. Amantidine HCl, benztropine mesylate, e.g., carbidopa.

Diuretic Drugs. Dichlorphenamide, methazolamide, bendroflumethiazide, polythiazide.

Antimigraine Drugs. Sumatriptan.

Hormones. Pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists, hormonal contraceptives, testicular hormones; gastrointestinal hormones: cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, sincalide; leptin.

Enzymes. Hyaluronidase, streptokinase, tissue plasminogen activator, urokinase, PGE-adenosine deaminase.

Intravenous Anesthetics. Droperidol, etomidate, fentanyl citrate/droperidol, hexobarbital, ketamine HCl, methohexital Na, thiamylal Na, thiopental Na.

Antiepileptics. Carbamazepine, clonazepam, divalproex Na, ethosuximide, mephenytoin, paramethadione, phenytoin, primidone.

Peptides and Proteins. The FcRn binding partners may be conjugated to peptides or polypeptides, e.g., ankyrins, arrestins, bacterial membrane proteins, clathrin, connexins, dystrophin, endothelin receptor, spectrin, selectin, cytokines, chemokines, growth factors, insulin, erythropoietin (EPO), tumor necrosis factor (TNF), CNTF, neuropeptides, neuropeptide Y, neurotensin, TGF-α, TGF-β, interferon (IFN), and hormones, growth inhibitors, e.g., genistein, steroids etc; glycoproteins, e.g., ABC transporters, platelet glycoproteins, GPIb-IX complex, GPIIb-IIIa complex, Factor VIa, Factor VIII, Factor IX, vitronectin, thrombomodulin, CD4, CD55, CD58, CD59, CD44, CD 152 (CTLA-4), lymphocye function-associated antigens (LFAs), intercellular adhesion molecules (ICAMs), vascular cell adhesion molecules (VCAMs), Thy-1, antiporters, CA-15-3 antigen, fibronectins, laminin, myelin-associated glycoprotein, GAP, GAP-43, and binding portions of receptors and counter-receptors for the above. In this embodiment of the present invention, the polypeptide therapeutics may be covalently conjugated to the FcRn binding partner, or the FcRn binding partner and therapeutic may be expressed as a fusion protein using standard recombinant genetic techniques.

Cytokines and Cytokine Receptors. Examples of cytokines and receptors thereof which may be delivered via an FcRn binding partner or conjugated to an FcRn binding partner in accordance with the present invention, include, but are not limited to: Interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor (LIF), M-CSF, PDGF, stem cell factor, transforming growth factor beta (TGF-β), TNF, TNFR, lymphotoxin, Fas, granulocyte colony-stimulating factor (G-CSF), GM-CSF, IFN-α, IFN-β, IFN-γ.

Growth Factors and Protein Hormones. Examples of growth factors and receptors thereof and protein hormones and receptors thereof which may be delivered via an FcRn binding partner or conjugated to an FcRn binding partner in accordance with the present invention, include, but are not limited to: EPO, angiogenin, hepatocyte growth factor, fibroblast growth factor, keratinocyte growth factor, nerve growth factor, tumor growth factor α, thrombopoietin (TPO), thyroid stimulating factor, thyroid releasing hormone, neurotrophin, epidermal growth factor, VEGF, ciliary neurotrophic factor, LDL, somatomedin, insulin growth factor, insulin-like growth factor I and II.

Chemokines. Examples of chemokines and receptors thereof which may be delivered via an FcRn binding partner or conjugated to an FcRn binding partner in accordance with the present invention, include, but are not limited to: ENA-78, ELC, GRO-α, GRO-β, GRO-γ, HRG, LIF, IP-10, MCP-1, MCP-2, MCP-3, MCP-4, MIP-1α, MIP-1β, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2, α-chemokine receptors: CXCR1, CXCR2, CXCR3, CXCR4, CXCR5, CXCR6, CXCR7, β-chemokine receptors: CCR1, CCR2, CCR3, CCR4, CCR5, CCR6, CCR7.

Chemotherapeutics. The FcRn binding partners may be conjugated to chemotherapy or anti-tumor agents which are effective against various types of human and other cancers, including leukemia, lymphomas, carcinomas, sarcomas, myelomas etc., such as, doxorubicin, mitomycin, cisplatin, daunorubicin, bleomycin, actinomycin D, neocarzinostatin, vinblastine, vincristine, taxol.

Antiviral Agents. The FcRn binding partners may be conjugated to antiviral agents such as reverse transcriptase inhibitors and nucleoside analogs, e.g., ddI, ddC, 3TC, ddA, AZT; protease inhibitors, e.g., Invirase, ABT-538; inhibitors of in RNA processing, e.g., ribavirin; and inhibitors of cell fusion, e.g., T-20 (Kilby J M et al. (1998) Nat Med. 4:1302-7).

Nucleic Acids. The FcRn binding partners may be conjugated to nucleic acid molecules such as antisense oligonucleotides and gene replacement nucleic acids. In certain embodiments involving conjugates with nucleic acids, a cleavable linker is included between the nucleic acid and the FcRn binding partner so that the nucleic acid can be available intracellularly. Antisense oligonucleotides include, for example and without limitation, anti-PKC-α, anti-ICAM-1, anti-H-ras, anti-Raf, anti-TNF-α, anti-VLA-4, anti-clusterin (all from Isis Pharmaceuticals, Inc.) and anti-Bc1-2 (GENASENSE™; Genta, Inc.).

Specific examples of known therapeutics which can be delivered via an FcRn binding partner include, but are not limited to:

(a) Capoten, Monopril, Pravachol, Avapro, Plavix, Cefzil, Duricef/Ultracef, Azactam, Videx, Zerit, Maxipime, VePesid, Paraplatin, Platinol, Taxol, UFT, Buspar, Serzone, Stadol NS, Estrace, Glucophage (Bristol-Myers Squibb);

(b) Ceclor, Lorabid, Dynabac, Prozac, Darvon, Permax, Zyprexa, Humalog, Axid, Gemzar, Evista (Eli Lilly);

(c) Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin (Merck & Co.);

(d) Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor (Pfizer);

(e) Vantin, Rescriptor, Vistide, Genotropin, Micronase/Glyn./Glyb., Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine (Pharmacia & Upjohn);

(f) Lopid, Accrupil, Dilantin, Cognex, Neurontin, Loestrin, Dilzem, Fempatch, Estrostep, Rezulin, Lipitor, Omnicef, FemHRT, Suramin, Clinafloxacin (Warner Lambert).

Further examples of therapeutic agents which can be delivered by the FcRn binding partners of the present invention may be found in Goodman and Gilman 's The Pharmacological Basis of Therapeutics, 9th ed., McGraw-Hill 1996, incorporated herein by reference in its entirety.

In one aspect of the invention, a method is provided for systemic delivery of an antibody to a subject. The method involves administering to a central airway of a subject an antibody in an aerosol, wherein a central lung zone/peripheral lung zone deposition ratio (C/P ratio) is at least 0.7, in an effective amount to achieve systemic delivery of the antibody to the subject. Systemic delivery of an antibody involves achievement of measurable amounts of the antibody in serum or tissue apart from the immediate site of administration. Confirmation of systemic delivery can be made using any suitable technique for measuring the presence or amount of the antibody in serum or a tissue.

As used herein, “antibody” refers generally to an antibody that is capable of binding to and transcellular transport by the FcRn receptor. As is well known in the art, antibodies are categorized by their structure into one of several classes or isotypes, namely IgG, IgA, IgM, IgE, and IgD. Certain of these classes have closely related subclasses (subtypes), e.g., IgG1, IgG2, IgG3, and IgG4 in humans. The FcRn receptor is believed to bind and transport antibodies of the IgG class. IgG antibodies generally have a molecular weight of ca. 150 kDa. In one embodiment the antibody is an IgG.

As is well known in the art, IgG antibodies as they occur in nature are bivalent glycoprotein molecules composed of two heavy chain polypeptides and two light chain polypeptides. Each heavy chain includes a variable domain (V_{H}), which participates in defining antigen specificity, and a constant domain (C_{H}) which is shared in common with other IgGs of the same subclass. The C_{H} domain in turn includes C_{H}1, hinge, C_{H}2, and C_{H}3 domains. Together, the hinge, C_{H}2, and C_{H}3 domains form a Fc fragment. Light chains are classified as either kappa (κ) or lambda (λ) light chains. Each light chain includes a variable domain (V_{L}), which also participates in defining antigen specificity of the antibody, and a constant domain (C_{L}). Together, paired V_{L}, C_{L}, and V_{H}, C_{H}1 domains form an antigen-binding fragment (Fab fragment). Proteolytic cleavage of an IgG antibody into two Fab fragments and one Fc fragment can be accomplished by papain digestion. See, for example, Abbas A K et al., Cellular and Molecular Immunology, 5^{th} Ed., W. B. Saunders: Philadelphia, 2003, pp 43-64.

In one embodiment the antibody includes an FcRn binding domain. As used herein, an “FcRn binding domain” refers to an antigen-nonspecific portion of an antibody which binds to an FcRn receptor. As described further herein, certain specific amino acid residues naturally present within the Fc domain of IgG have been reported to be involved in antibody-FcRn interaction.

In one embodiment the antibody includes a human Fc fragment. More specifically, in one embodiment the antibody includes an Fc fragment of a human IgG. Such an antibody can be a fully human antibody, a chimeric antibody having a human Fc fragment, or a humanized antibody, as described in further detail below. In a particular embodiment the antibody includes a human IgG1 Fc fragment (human Fcγ1; e.g., as provided by SEQ ID NO: 2).

In one embodiment the antibody is a monoclonal antibody. Monoclonal antibodies and methods for their preparation are well known in the art, beginning with the original description in 1975 by Kohler and Milstein. As used herein, monoclonal antibodies include engineered antibodies such as chimeric and humanized antibodies. Examples of monoclonal antibodies useful in therapeutic, diagnostic, and other applications are too numerous to recount. Therapeutic and diagnostic monoclonal antibodies include those already in clinical use, as well as those in development for clinical use. Examples of therapeutic monoclonal antibodies already in clinical use include those shown in Table 1.

TABLE 1
Therapeutic monoclonal antibodies in current clinical use.

| TRADE NAME | GENERIC NAME | MANUFACTURER | TARGET |
|---|---|---|---|
| CAMPATH ® | Alemtuzumab | ILEX/Millennium | CD52 |
| HERCEPTIN ® | Trastuzumab | Genentech | HER2 |
| HUMIRA ™ | Adalimumab | Abbott | TNF-α |
| OKT ®3 | Muromonab-CD3 | Ortho Biotech | CD3 |
| RAPTIVA | Efalizumab | XOMA/Genentech | CD11a |
| REMICADE ® | Infliximab | Centocor | TNF-α |
| RITUXAN ® | Rituximab | IDEC/Genentech | CD20 |
| SIMULECT ® | Basiliximab | Novartis | CD25 |
| SYNAGIS ® | Palivizumab | MedImmune | RSV |
| ZENAPAX ® | Daclizumab | Hoffman-LaRoche | CD25 |
| ZEVALIN ™ | Ibritumomab | IDEC | CD20 |
| | tiuxetan | | |

Other therapeutic antibodies in current clinical use are Fab fragments of whole antibodies and are not included in the table.

In another embodiment the antibody is an immune globulin or a hyperimmune globulin. These are polyclonal antibody preparations derived from subjects previously exposed to an antigen or antigens of interest. They can be used to passively immunize a subject by supplying the subject with a source of antibodies when the treated subject cannot form his own antibodies in sufficient quantity or cannot form his own antibodies in a sufficiently short time. Examples of therapeutic immune globulin and hyperimmune globulin in clinical use include those shown in Table 2.

TABLE 2
Immune globulin and hyperimmune globulin in current clinical use.

| | | MANU- | |
|---|---|---|---|
| TRADE NAME | CATEGORY | FACTURER | TARGET |
| BAYGAM ® | immune | Bayer | viruses |
| | globulin | Biological | |
| BAYHEP B ® | hyperimmune | Bayer | HBsAg |
| | globulin | Biological | |
| BAYRAB ® | hyperimmune | Bayer | rabies virus |
| | globulin | Biological | |
| BAYTET ® | hyperimmune | Bayer | tetanus toxin |
| | globulin | Biological | |
| CYTOGAM ® | immune | MedImmune | CMV |
| | globulin | | |
| GAMIMUNE N ® | immune | Bayer | general |
| | globulin | Biological | |
| IMOGAM ® RABIES | hyperimmune | Aventis Pasteur | rabies virus |
| | globulin | | |
| NABI-HB ™ | hyperimmune | Nabi | HBsAg |
| | globulin | | |
| RESPIGAM ® | hyperimmune | MedImmune | RSV |
| | globulin | | |
| SANDOGLOBULIN ® | immune | Novartis | general |
| | globulin | | |
| WINRHO SDF ™ | immune | Nabi | Rho D Ag |
| | globulin | | |

Human FcRn binds to all subclasses of human IgG but not as well to most subclasses of IgG from other species, e.g., mouse and rat IgG. West A P et al. (2000) Biochemistry 39:9698-9708; Ober R J et al. (2001) Int Immunol 13:1551-59. Thus in certain embodiments the species of the subject to be treated corresponds to the species of origin of IgG from which FcRn binding partners can be derived. The order of affinities of binding within each species is IgG1=IgG2>IgG3>IgG4 (human); IgG1>IgG2b>IgG2a>IgG3 (mouse); and IgG2a>IgG1>IgG2b=IgG2c (rat). Burmeister W P et al (1994) Nature 372:379-83. It is believed, therefore, that human IgG (and FcRn contact-containing fragments thereof) belonging to any subclass is useful as a human FcRn binding partner. Accordingly, for human subjects in one embodiment the antibody is a human IgG1. Alternatively, for human subjects in one embodiment the antibody is a human IgG2. In yet other separate embodiments the antibody is a human IgG3 or a human IgG4.

More specifically, in certain embodiments in which the subject is a human, the antibody may be a fully human antibody, a chimeric antibody, or a humanized antibody. Importantly, the FcRn receptor-binding domain of the antibody may be of human origin or it may mimic an FcRn receptor-binding domain of a human antibody. For example, a fully human antibody includes a human Fc domain which naturally binds the FcRn receptor. A chimeric antibody is a genetically engineered form of monoclonal antibody that typically includes at least human constant heavy chains (CH, which include the Fc domain) and non-human antigen-binding domains, e.g., murine variable heavy (V_{H}) and variable light (V_{L}) chains. A humanized antibody is a genetically engineered form of monoclonal antibody that typically includes human constant heavy and constant light chains, human heavy and light chain variable domain framework, and minimal non-human sequence defining antigen-contacting residues (complementarity-determining regions, CDRs). As suggested above, in one embodiment the antibody may be of non-human origin but will include human IgG residues corresponding to residues 248, 250-257, 272, 285, 288, 290-291, 307, 308-311 and 314 in C_{H}² and 385-387, 428 and 433-436 in C_{H}3. In yet other embodiments the antibody may be of non-human origin but will include at least one human IgG residue corresponding to any one of residues 248, 250-257, 272, 285, 288, 290-291, 307, 308-311 and 314 in C_{H}2 and 385-387, 428 and 433-436 in C^{H}3.

In one embodiment the antibody to be administered to a central airway of a subject is a therapeutic antibody. As used herein, “therapeutic antibody” refers to an antibody useful to treat a disease or condition of a subject. Without meaning to be bound by any particular mechanism, a therapeutic antibody may exert its effect by binding to its target molecule (antigen), thereby neutralizing the biological effect or enhancing removal of the target molecule, or by directing immune cell- or complement-mediated killing of a cell expressing the antigen. Examples of therapeutic antibodies include, without limitation, anti-CD52, anti-CD25, anti-TNF-α, anti-RSV, anti-CD20, anti-HER2, and anti-CEA. Such antibodies specifically may include CAMPATH®, SIMULECT®, ZENAPAX®, REMICADE®, HUMIRA™, SYNAGIS®, RITUXAN®, HERCEPTIN™, and CEA-CIDE™.

Respiratory syncytial virus (RSV) is the leading cause of serious lower respiratory tract disease in infants and children. Feigen et al., eds., 1987, In: Textbook of Pediatric Infectious Diseases, W B Saunders, Philadelphia, pp. 1653-75; New Vaccine Development, Establishing Priorities, Vol. 1, 1985, National Academy Press, Washington D.C., pp. 397-409; and Ruuskanen O et al. (1993) Curr Probl Pediatr 23:50-79. The yearly epidemic nature of RSV infection is evident worldwide, but the incidence and severity of RSV disease in a given season vary by region. Hall C B (1993) Contemp Pediatr 10:92-110. In temperate regions of the northern hemisphere, it usually begins in late fall and ends in late spring. Primary RSV infection occurs most often in children from 6 weeks to 2 years of age and uncommonly in the first 4 weeks of life during nosocomial epidemics. Hall C B et al. (1979) New Engl J Med 300:393-6. Children at increased risk of RSV infection include preterm infants (Hall C B et al. (1979) New Engl J Med 300:393-6) and children with bronchopulmonary dysplasia (Groothuis J R et al. (1988) Pediatrics 82:199-203), congenital heart disease (MacDonald N E et al. (1982) New Engl J Med 307:397-400), congenital or acquired immunodeficiency (Ogra P L et al. (1988) Pediatr Infect Dis J 7:246-9; Pohl C et al. (1992) J Infect Dis 165:166-9), and cystic fibrosis (Abman S H et al. (1988) J Pediatr 113:826-30). The fatality rate in infants with heart or lung disease who are hospitalized with RSV infection is 3%-4%. Navas L et al. (1992) J Pediatr 121:348-54).

RSV infects adults as well as infants and children. In healthy adults, RSV causes predominantly upper respiratory tract disease. It has recently become evident that some adults, especially the elderly, have symptomatic RSV infections more frequently than had been previously reported. Evans, A. S., ed., 1989, Viral Infections of Humans. Epidemiology and Control, 3rd ed., Plenum Medical Book, New York, pp. 525-544). Several epidemics also have been reported among nursing home patients and institutionalized young adults. Falsey A R (1991) Infect Control Hosp Epidemiol 12:602-8; Garvie D G et al. (1980) Br Med J 281:1253-4. Finally, RSV may cause serious disease in immunosuppressed persons, particularly bone marrow transplant recipients. Hertz M I et al. (1989) Medicine 68:269-81.

Treatment options for established RSV disease are limited. Severe RSV disease of the lower respiratory tract often requires considerable supportive care, including administration of humidified oxygen and respiratory assistance. Fields et al., eds, 1990, Fields Virology, 2d ed., Vol. 1, Raven Press, New York, pp. 1045-72. The only drug approved for treatment of infection is the antiviral agent ribavirin. American Academy of Pediatrics Committee on Infectious Diseases (1993) Pediatrics 92:501-4. It has been shown to be effective in the treatment of RSV pneumonia and bronchiolitis, modifying the course of severe RSV disease in immunocompetent children. Smith D W et al. (1991) New Engl J Med 325:24-9. However, ribavirin has had limited use because it requires prolonged aerosol administration and because of concerns about its potential risk to pregnant women who may be exposed to the drug during its administration in hospital settings.

A humanized monoclonal antibody directed to an epitope in the A antigenic site of the F protein of RSV, SYNAGIS® (Palivizumab, Med Immune), is currently approved in the United States for intramuscular administration to pediatric patients for prevention of serious lower respiratory tract disease caused by RSV at recommended monthly doses of 15 mg/kg of body weight throughout the RSV season (November through April in the northern hemisphere). SYNAGIS® is a composite of human (95%) and murine (5%) antibody sequences. Johnson S et al. (1997) J Infect Dis 176:1215-24; and U.S. Pat. No. 5,824,307, the entire contents of which are incorporated herein by reference. The human heavy chain sequence was derived from the constant domains of human IgG1 and the variable framework regions of the V_{H} genes of Cor (Press E M et al. (1970) Biochem J 117:641-60) and CESS (Takashi N et al. (1984) Proc Natl Acad Sci USA 81:5194-8). The human light chain sequence was derived from the constant domain of Cκ and the variable framework regions of the V_{L} gene K104 with Jκ-4. Bentley D L et al. (1980) Nature 288:730-3. The murine sequences were derived from a murine monoclonal antibody, Mab 1129 (Beeler J A et al. (1989) J Virol 63:2941-50), in a process which involved the grafting of the murine complementarity-determining regions (CDRs) into the human antibody frameworks. SYNAGIS® is composed of two heavy chains and two light chains and has a molecular weight of approximately 148 kDa. SYNAGIS® exhibits both neutralizing and fusion-inhibitory activity against RSV.

Although SYNAGIS® has been successfully used for the prevention of RSV infection in pediatric patients, multiple intramuscular doses of 15 mg/kg of SYNAGIS® is required to achieve a prophylactic effect. In pediatric patients less than 24 months of age, the mean half-life of SYNAGIS® has been shown to be 20 days and monthly intramuscular doses of 15 mg/kg have been shown to result in a mean i standard derivation 30 day serum titer of 37±21 μg/ml after the first injection, 57±41 μg/ml after the second injection, 68±51 μg/ml after the third injection, and 72±50 μg/ml after the fourth injection. The Impact-RSV Study Group (1998) Pediatrics 102:531-7. Serum concentrations of greater than 30 μg/ml have been shown to be necessary to reduce pulmonary RSV replication by 100 fold in the cotton rat model of RSV infection. However, the administration of multiple intramuscular doses of 15 mg/kg of antibody is inconvenient for the patient.

REMICADE® (Infliximab, Centocor) is a chimeric IgG1, κ monoclonal antibody with an approximate molecular weight of 149 kDa. It is composed of human constant and murine variable regions. Infliximab binds specifically to human tumor necrosis factor alpha (TNF-α) with an association constant of 10¹⁰ M⁻¹.

REMICADE®, in combination with methotrexate, is indicated for reducing signs and symptoms, inhibiting the progression of structural damage and improving physical function in patients with moderately to severely active rheumatoid arthritis who have had an inadequate response to methotrexate. The recommended dose of REMICADE® is 3 mg/kg given as an intravenous infusion followed with additional similar doses at 2 and 6 weeks after the first infusion then every 8 weeks thereafter. REMICADE® is normally given in combination with methotrexate. For patients who have an incomplete response, the dose may be adjusted up to 10 mg/kg or the dosing schedule may be adjusted up to as often as every 4 weeks.

REMICADE® is also indicated for the reduction in signs and symptoms of Crohn's disease in patients with moderately to severely active Crohn's disease who have had an inadequate response to conventional therapy. The recommended dose of REMICADE® is 5 mg/kg given as a single intravenous infusion for treatment of moderately to severely active Crohn's disease. In patients with fistulizing disease, an initial 5 mg/kg dose is usually followed with additional 5 mg/kg doses at 2 and 6 weeks after the first infusion.

Infliximab neutralizes the biological activity of TNF-α by binding with high affinity to the soluble and transmembrane forms of TNF-α and inhibits binding of TNF-α with its receptors. Knight D M et al. (1993) Molec Immunol 30:1443-53; Scallon B J et al. (1995) Cytokine 7:251-9; Siegel S A et al. (1995) Cytokine 7:15-25. Infliximab does not neutralize TNF-β (lymphotoxin-α), a related cytokine that utilizes the same receptors as TNF-α. Biological activities attributed to TNF-α include: induction of pro-inflammatory cytokines such as interleukins IL-1 and IL-6, enhancement of leukocyte migration by increasing endothelial layer permeability and expression of adhesion molecules by endothelial cells and leukocytes, activation of neutrophil and eosinophil functional activity, induction of acute phase reactants and other liver proteins, as well as tissue degrading enzymes produced by synoviocytes and/or chondrocytes. Cells expressing transmembrane TNF-α bound by Infliximab can be lysed in vitro by complement or effector cells. Scallon B J et al. (1995) Cytokine 7:251-9. Infliximab inhibits the functional activity of TNF-α in a wide variety of in vitro bioassays utilizing human fibroblasts, endothelial cells, neutrophils (Siegel S A et al. (1995) Cytokine 7:15-25) B and T lymphocytes and epithelial cells. Anti-TNF-α antibodies reduce disease activity in the cotton-top tamarin colitis model, and decrease synovitis and joint erosions in a murine model of collagen-induced arthritis. Infliximab prevents disease in transgenic mice that develop polyarthritis as a result of constitutive expression of human TNF-α, and, when administered after disease onset, allows eroded joints to heal.

HUMIRA™ (Adalimumab, Abbott) is a recently FDA-approved recombinant human IgG1 monoclonal antibody specific for human TNF-α. HUMIRA™ was created using phage display technology resulting in an antibody with human-derived heavy and light chain variable regions and human IgG1, κ constant regions. HUMIRA™ is produced by recombinant DNA technology in a mammalian cell expression system and is purified by a process that includes specific viral inactivation and removal steps. It consists of 1330 amino acids and has a molecular weight of approximately 148 kDa.

Adalimumab binds specifically to TNF-α and blocks its interaction with the p55 and p75 cell surface TNF receptors. Adalimumab also lyses surface TNF-expressing cells in vitro in the presence of complement. Adalimumab does not bind or inactivate lymphotoxin (TNF-β). Elevated levels of TNF-α are found in the synovial fluid of rheumatoid arthritis patients and play an important role in both the pathologic inflammation and the joint destruction that are hallmarks of rheumatoid arthritis.

Adalimumab also modulates biological responses that are induced or regulated by TNF-α, including changes in the levels of adhesion molecules responsible for leukocyte migration (ELAM-1, VCAM-1, and ICAM-1 with an IC₅₀ of 1-2×10⁻¹⁰M).

Adalimumab mean steady-state trough concentrations of approximately 5 μg/ml and 8 to 9 μg/ml were observed without and with methotrexate, respectively. The serum adalimumab trough levels at steady state increase approximately proportionally with dose following 20, 40 and 80 mg every other week and every week subcutaneous dosing.

HUMIRA™ is currently approved for use in the United States for reducing signs and symptoms and inhibiting the progression of structural damage in adult patients with moderately to severely active rheumatoid arthritis who have had an inadequate response to one or more disease-modifying antirheumatic drugs (DMARDs). HUMIRA™ can be used alone or in combination with methotrexate or other DMARDs.

The recommended dose of HUMIRA™ for adult patients with rheumatoid arthritis is 40 mg administered every other week as a subcutaneous injection. Methotrexate, glucocorticoids, salicylates, nonsteroidal anti-inflammatory drugs (NSAIDs), analgesics, or other DMARDs can be continued during treatment with HUMIRA™. Some patients not taking concomitant methotrexate may derive additional benefit from increasing the dosing frequency of HUMIRA™ to 40 mg every week.

SIMULECT® (Basiliximab, Novartis) is a chimeric (murine/human) monoclonal antibody (IgG1) produced by recombinant DNA technology, that functions as an immunosuppressive agent, specifically binding to and blocking the interleukin-2 receptor alpha-chain (IL-2Rα, also known as CD25 antigen) on the surface of activated T-lymphocytes. Based on the amino acid sequence, the calculated molecular weight of the protein is 144 kDa. It is a glycoprotein obtained from fermentation of an established mouse myeloma cell line genetically engineered to express plasmids containing the human heavy and light chain constant region genes and mouse heavy and light chain variable region genes encoding the RFT5 antibody that binds selectively to the IL-2Rα.

Basiliximab is currently indicated for the prophylaxis of acute organ rejection in patients receiving renal transplantation when used as part of an immunosuppressive regimen that includes cyclosporine and corticosteroids. Basiliximab may also be useful for the treatment and prophylaxis of acute organ rejection in patients receiving solid organ and bone marrow allografts. In addition, Basiliximab may be useful for treatment of tumors expressing CD25, e.g., T-cell leukemia/lymphoma.

Basiliximab functions as an IL-2 receptor antagonist by binding with high affinity (Kₐ=1×10¹⁰ M⁻¹) to the alpha chain of the high affinity IL-2 receptor complex and inhibiting IL-2 binding. Basiliximab is specifically targeted against IL-2Rα, which is selectively expressed on the surface of activated T-lymphocytes. This specific high affinity binding of SIMULECT® to IL-2Rα competitively inhibits IL-2-mediated activation of lymphocytes, a critical pathway in the cellular immune response involved in allograft rejection. While in the circulation, SIMULECT® impairs the response of the immune system to antigenic challenges.

In adult renal allograft recipient patients, the recommended regimen is two doses of 20 mg each. The first 20 mg dose typically is given within 2 hours prior to transplantation surgery. The recommended second 20 mg dose typically is given 4 days after transplantation. In pediatric renal allograft recipient patients weighing less than 35 kg, the recommended 30 regimen is two doses of 10 mg each. In pediatric patients weighing 35 kg or more, the recommended regimen is two doses of 20 mg each. The first dose typically is given within 2 hours prior to transplantation surgery. The recommended second dose typically is given 4 days after transplantation.

ZENAPAX® (Daclizumab, Roche) is an immunosuppressive, humanized IgG1 monoclonal antibody produced by recombinant DNA technology that binds specifically to the alpha subunit (p55, alpha, CD25, or Tac subunit) of the human high-affinity interleukin-2 (IL-2) receptor that is expressed on the surface of activated lymphocytes. Daclizumab is a composite of human (90%) and murine (10%) antibody sequences. The human sequences were derived from the constant domains of human IgG1 and the variable framework regions of the Eu myeloma antibody. The murine sequences were derived from the complementarity-determining regions of a murine anti-Tac antibody. The molecular weight predicted from DNA sequencing is 144 kDa.

Like Basiliximab, Daclizumab is currently indicated for the prophylaxis of acute organ rejection in patients receiving renal transplants. It is used as part of an immunosuppressive regimen that includes cyclosporine and corticosteroids. Likewise, Daclizumab may also be useful for the treatment and prophylaxis of acute organ rejection in patients receiving solid organ and bone marrow allografts, as well as for treatment of tumors expressing CD25, e.g., T-cell leukemia/lymphoma.

In renal allograft recipients, the recommended dose for Daclizumab is 1.0 mg/kg. Based on the clinical trials, the standard course of ZENAPAX therapy is five doses. The first dose normally is given no more than 24 hours before transplantation. The four remaining doses normally are given at intervals of 14 days.

CAMPATH® (Alemtuzumab, ILEX/Millennium) is a recombinant DNA-derived humanized monoclonal antibody (Campath-1H) that is directed against the 21-28 kDa cell surface glycoprotein, CD52. CD52 is expressed on the surface of normal and malignant B and T lymphocytes, NK cells, monocytes, macrophages, and tissues of the male reproductive system. The Campath-1H antibody is an IgG1, κ with human variable framework and constant regions, and complementarity-determining regions from a murine (rat) monoclonal antibody (Campath-1G). The Campath-1H antibody has an approximate molecular weight of 150 kDa.

Alemtuzumab is indicated for the treatment of B-cell chronic lymphocytic leukemia (B-CLL) in patients who have been treated with alkylating agents and who have failed fludarabine therapy.

Alemtuzumab binds to CD52, a non-modulating antigen that is present on the surface of essentially all B and T lymphocytes, a majority of monocytes, macrophages, and NK cells, and a subpopulation of granulocytes. Analysis of samples collected from multiple volunteers has not identified CD52 expression on erythrocytes or hematopoetic stem cells. The proposed mechanism of action is antibody-dependent lysis of leukemic cells following cell surface binding. Campath-1H Fab binding was observed in lymphoid tissues and the mononuclear phagocyte system. A proportion of bone marrow cells, including some CD34⁺ cells, express variable levels of CD52. Significant binding was also observed in the skin and male reproductive tract (epididymis, sperm, seminal vesicle). Mature spermatozoa stain for CD52, but neither spermatogenic cells nor immature spermatozoa show evidence of staining.

Campath therapy is typically initiated at a dose of 3 mg administered as a 2 hour IV infusion daily. Doses are increased as tolerated until the maintenance dose of Campath is 30 mg/day can be administered as a 2 hour IV infusion three times per week on alternate days for up to 12 weeks. In most patients, escalation to 30 mg is accomplished in 3-7 days.

RITUXAN® (Rituximab; IDEC/Genentech) is a genetically engineered chimeric murine/human monoclonal antibody directed against the CD20 antigen found on the surface of normal and malignant B lymphocytes. Rituximab is indicated for the treatment of patients with relapsed or refractory low-grade or follicular, CD20 positive, B-cell non-Hodgkin's lymphoma. The antibody is an IgG1,κ immunoglobulin containing murine light- and heavy-chain variable region sequences and human constant region sequences. Rituximab is composed of two heavy chains of 451 amino acids and two light chains of 213 amino acids (based on cDNA analysis) and has an approximate molecular weight of 145 kDa. Rituximab has a binding affinity for the CD20 antigen of approximately 8.0 nM.

Rituximab binds specifically to the antigen CD20 (human B-lymphocyte-restricted differentiation antigen, Bp35), a hydrophobic transmembrane protein with a molecular weight of approximately 35 kD located on pre-B and mature B lymphocytes. Valentine M A et al. (1989) J Biol Chem 264:11282-7; Einfeld D A et al. (1988) EMBO J 7:711-7. The antigen is also expressed on >90% of B-cell non-Hodgkin's lymphomas (NHL; Anderson K C et al. (1984) Blood 63:1424-33) but is not found on hematopoietic stem cells, pro-B cells, normal plasma cells or other normal tissues. Tedder T F et al. (1985) J Immunol 135:973-9. CD20 regulates an early step(s) in the activation process for cell cycle initiation and differentiation (Tedder T F et al. (1985) J Immunol 135:973-9), and possibly functions as a calcium ion channel. Tedder T F et al. (1990) J Cell Biochem 14D: 195. CD20 is not shed from the cell surface and does not internalize upon antibody binding. Press O W et al. (1987) Blood 69:584-91. Free CD20 antigen is not found in the circulation. Einfeld D A et al. (1988) EMBO J 7:711-7.

5 The Fab domain of Rituximab binds to the CD20 antigen on B lymphocytes, and the Fc domain recruits immune effector functions to mediate B-cell lysis in vitro. Possible mechanisms of cell lysis include complement-dependent cytotoxicity (CDC; Reff M E et al. (1994) Blood 83:435-45) and antibody-dependent cell mediated cytotoxicity (ADCC). The antibody has been shown to induce apoptosis in the DHL-4 human B-cell lymphoma line. Demidem A et al. (1997) Cancer Biother Radiopharm 12:177-86.

The recommended dosage of RITUXAN® is 375 mg/m² given as an IV infusion once weekly for four doses (Days 1, 8, 15, and 22).

HERCEPTIN® (Trastuzumab; Genentech) is a recombinant DNA-derived humanized monoclonal antibody that selectively binds with high affinity in a cell-based assay (Kd=5 nM) to the extracellular domain of the human epidermal growth factor receptor 2 protein, HER2. Coussens L et al. (1985) Science 230:1132-9; Slamon D J et al. (1989) Science 244:707-12. Trastuzumab as a single agent is indicated for the treatment of patients with metastatic breast cancer whose tumors overexpress the HER2 protein and who have received one or more chemotherapy regimens for their metastatic disease. Trastuzumab in combination with paclitaxel is indicated for treatment of patients with metastatic breast cancer whose tumors overexpress the HER2 protein and who have not received chemotherapy for their metastatic disease. The antibody is an IgG1, κ that contains human framework regions with the complementarity-determining regions of a murine antibody (4D5) that binds to HER2.

The HER2 (or c-erbB2) proto-oncogene encodes a transmembrane receptor protein of 185 kDa, which is structurally related to the epidermal growth factor receptor. Coussens L et al. (1985) Science 230:1132-9. HER2 protein overexpression is observed in 25%-30% of primary breast cancers. HER2 protein overexpression can be determined using an immunohistochemistry-based assessment of fixed tumor blocks. Press M F et al. (1993) Cancer Res 53:4960-70.

Trastuzumab has been shown, in both in vitro assays and in animals, to inhibit the proliferation of human tumor cells that overexpress HER2. Hudziak K M et al. (1989) Mol Cell Biol 9:1165-72; Lewis G D et al. (1993) Cancer Immunol Immunother 37:255-63; Baselga J et al. (1998) Cancer Res 58: 2825-31. Trastuzumab is a mediator of antibody-dependent cellular cytotoxicity (ADCC). Hotaling T E et al. (1996) Proc Annu Meet Am Assoc Cancer Res 37:471; Pegram M D et al. (1997) Proc Am Assoc Cancer Res 38:602. In vitro, Trastuzumab-mediated ADCC has been shown to be preferentially exerted on HER2-overexpressing cancer cells compared with cancer cells that do not overexpress HER2.

The recommended initial loading dose is 4 mg/kg Trastuzumab administered as a 90-minute infusion. The recommended weekly maintenance dose is 2 mg/kg Trastuzumab and can be administered as a 30-minute infusion if the initial loading dose was well tolerated.

CEA-CIDE™ (Labetuzumab, Immunomedics) is humanized monoclonal antibody against carcinoembryonic antigen (CEA), in which about 90% all murine components in the antibody have been replaced with human immunoglobulin structures. This antibody is in clinical studies as a naked (unlabeled) and a radiolabeled conjugate, for the therapy of diverse cancers expressing CEA, including colorectal, pancreatic and breast cancers. Primary use of CEA-CIDE™ Naked humanized antibody is for the treatment of inoperable metastatic solid tumors. Every year there are 140,000 newly diagnosed and 65,000 deaths due to colorectal cancer; 180,000 new cases and 45,000 deaths due to breast cancer; 172,000 new case and 160,000 deaths due to lung cancer; 25,000 new case and 15,000 deaths due to ovarian cancer; and 29,000 new cases and 28,000 deaths due to pancreatic cancer. It is currently in Phase I clinical trials. CEA-CIDE Y-90™, an Yttrium-90-labeled form of Labetuzumab, also has as its primary use the treatment of inoperable metastatic solid tumors. It also is currently in Phase I clinical trials.

In another embodiment the antibody is a diagnostic antibody. As used herein, “diagnostic antibody” refers to an antibody useful for detecting or localizing a target associated with a disease or condition in a subject. The diagnostic antibody may in one embodiment be a diagnostic imaging antibody, e.g., an antibody linked to a radionuclide such as ^{99m}Tc, ^{113m}In, ¹³¹I, or ^{81m}Kr, a metal such as gadolinium, or a tag such as biotin, useful for detecting the antibody. In some embodiments a diagnostic antibody is also a therapeutic antibody. In diagnostic embodiments, the antibody may be linked to a pharmaceutically acceptable radioisotopes, including but not limited to, those of iodine, indium, technetium, and xenon; magnetic particles; a metal useful in magnetic resonance imaging (MRI; e.g., gadolinium); radio-opaque materials such as barium; and fluorescent compounds.

In another aspect the invention provides a method for passively immunizing a subject. The method according to this aspect of the invention involves administering to a central airway of a subject, wherein said subject is in need of passive immunization against an antigen, an antigen-specific antibody in an aerosol, wherein a central lung zone/peripheral lung zone deposition ratio (C/P ratio) is at least 0.7, in an effective amount to neutralize the antigen in the subject. As used herein, a subject in need of passive immunization against an antigen is a subject that has been exposed or is at risk of becoming exposed to an antigen and that cannot form his own antibodies against the antigen in sufficient quantity or in a sufficiently short time to protect the subject against the antigen. Such subjects include, for example, subjects with hypogammaglobulinemia, agammaglobulinemia, subjects receiving or recovering from immunosuppressive treatment, subjects receiving or recovering from marrow-suppressive chemotherapeutic or radiation treatment, subjects exposed or believed to be at risk of being exposed to certain viruses including rabies virus, cytomegalovirus (CMV), respiratory syncitial virus (RSV), hepatitis B virus (HBV, with specific antigen hepatitis B surface antigen, HbsAg), and subjects exposed or believed to be at risk of being exposed to other agents such as a microbial toxin. Traditionally such subjects are passively immunized by intramuscular or intravenous administration of an appropriate immune globulin or hyperimmune globulin.

The antibody is administered in an amount effective to neutralize the antigen in the subject. As used herein, “neutralize” refers to blockade of the biological effects of the antigen that normally would occur in the absence of the antibody. Thus neutralization of a toxin blocks the toxic effects of the toxin. Neutralization of a virus or other infectious agent blocks infectious process of the virus or other infectious agent.

In yet another aspect the invention provides a method for treating a deep lung disease in a subject. The method according to this aspect of the invention involves administering to a central airway of a subject, wherein said subject is in need of an antibody for treatment of a deep lung disease, an antibody in an aerosol, wherein a central lung zone/peripheral lung zone deposition ratio (C/P ratio) is at least 0.7, in an effective amount to treat the deep lung disease of the subject. As used herein, a “deep lung disease” refers to a disease involving an obstruction of, or an accumulation of fluid, cells, or infectious organisms within, airways of the lung distal to the central airways. Treatment of a deep lung disease generally can involve use of any of a number of suitable therapeutics, including but not limited to antibodies. As used herein, “a subject in need of an antibody for treatment of a deep lung disease” refers to a subject having or at risk of developing a disease in the deep lung for which is indicated treatment with an antibody. It is to be noted that the method according to this aspect of the invention calls for administration of the antibody to a central airway, rather than to the deep lung itself.

A classic deep lung disease is pneumonia. Accordingly, in one embodiment the deep lung disease is pneumonia, for example RSV pneumonia or CMV pneumonia. Deep lung disease also includes certain malignancies, either primary in the lung or metastatic to the lung, as well as extranodal pulmonary non-Hodgkin's lymphoma. In certain embodiments the antibody is any one of anti-RSV, anti-CMV, anti-CD52, anti-CD20, anti-HER2, and anti-CEA. In particular embodiments the antibody is any one of SYNAGIS®, CAMPATH®, RITUXAN®, HERCEPTIN®, and CEA-CIDE™.

As used herein, the term “to treat” means to ameliorate the signs or symptoms of; to slow, stop, or reverse the progression of; or to prevent the development of a disease, disorder, or condition of a subject. Signs, symptoms, and progression of a particular disease, disorder, or condition of a subject can be assessed using any applicable clinical or laboratory measure recognized by those of skill in the art, e.g., as described in Harrison 's Principles of Internal Medicine, 14^{th} Ed., Fauci A S et al., eds., McGraw-Hill, New York, 1998. As used herein, the term “subject” means a mammal. For treating or preventing a particular disease, disorder, or condition, those of skill in the art will recognize a suitable therapeutic agent, e.g., a particular antibody, for that purpose.

In another aspect the invention provides a method for treating extrapulmonary disease in a subject. The method according to this aspect of the invention involves administering to a central airway of a subject, wherein said subject is in need of an antibody for treatment of extrapulmonary disease, an antibody in an aerosol, wherein a central lung zone/peripheral lung zone deposition ratio (C/P ratio) is at least 0.7, in an effective amount to treat the extrapulmonary disease of the subject. As used herein, an “extrapulmonary disease” refers to any disease that involves a non-pulmonary tissue or organ. A non-pulmonary tissue or organ includes, without limitation, skin, muscle, bone, synovium, marrow, blood, lymphatics, brain, eye, heart, esophagus, stomach, intestine, gall bladder, liver, pancreas, spleen, kidney, uterus, ovary, testis. Treatment of an extrapulmonary disease generally can involve use of any of a number of suitable therapeutics, including but not limited to antibodies. As used herein, “a subject in need of an antibody for treatment of extrapulmonary disease” refers to subject having or at risk of developing a disease outside the lung, e.g., involving a non-pulmonary tissue or organ, for which treatment with an antibody is indicated. In one embodiment the subject may also have or be at risk of developing disease involving the lung as an aspect of the same disease occurring apart from the lung.

In one embodiment the extrapulmonary disease is cancer. Where the extrapulmonary disease is cancer, in certain embodiments the antibody is chosen from anti-CD52, anti-CD25, anti-CD20, anti-HER2, and anti-CEA. In particular, in certain embodiments the antibody is chosen from CAMPATH®, SIMULECT®, ZENAPAX®, RITUXAN®, HERCEPTIN®, and CEA-CIDE™.

In another embodiment according to this aspect of the invention, the extrapulmonary disease is an autoimmune disease. Autoimmune diseases include without limitation those listed above in reference to antigens characteristic of an autoimmune disease. In one embodiment the autoimmune disease is rheumatoid arthritis. In another embodiment the autoimmune disease is Crohn's disease. Where the extrapulmonary disease is an autoimmune disease, in one embodiment the antibody is anti-TNF-α. In a particular embodiment, the antibody is REMICADE®. In another particular embodiment, the antibody is HUMIRA™.

In another embodiment according to this aspect of the invention, the extrapulmonary disease is non-pulmonary allograft rejection. As used herein, “non-pulmonary allograft rejection” refers to immune rejection of an organ or tissue, other than lung, transplanted from one individual into another individual. For example, the allograft may be a kidney, a liver or a portion thereof, a heart, a pancreas, pancreatic islets, small intestine, skin, bone marrow, neural tissue, or a limb or portion thereof. Typically the rejection is acute rejection, but the rejection may be hyperacute or chronic rejection. Where the extrapulmonary disease is non-pulmonary allograft rejection, in one embodiment the antibody is anti-CD25. In a particular embodiment, the antibody is selected from SIMULECT® and ZENAPAX®.

When administered, the antibodies and conjugates of the present invention are administered in pharmaceutically acceptable preparations. Such preparations can routinely contain pharmaceutically acceptable concentrations of salt, buffering agents, preservatives, compatible carriers, supplementary immune potentiating agents such as adjuvants and cytokines, and optionally other therapeutic agents. Thus, “cocktails” including the antibodies or conjugates and the other agents are contemplated. The therapeutic agents themselves are conjugated to FcRn binding partners to enhance delivery of the therapeutic agents across the pulmonary epithelial barrier.

The antibodies and conjugates of the invention can be administered in a purified form or in the form of a pharmaceutically acceptable salt. When used in medicine the salts should be pharmaceutically acceptable, but non-pharmaceutically acceptable salts may conveniently be used to prepare pharmaceutically acceptable salts thereof and are not excluded from the scope of the invention. Such pharmaceutically acceptable salts include, but are not limited to, those prepared from the following acids: hydrochloric, hydrobromic, sulfuric, nitric, phosphoric, maleic, acetic, salicylic, p-toluene sulfonic, tartaric, citric, methane sulfonic, formic, malonic, succinic, naphthalene-2-sulfonic, and benzene sulfonic. Also, pharmaceutically acceptable salts can be prepared as alkaline metal or alkaline earth salts, such as sodium, potassium or calcium salts of the carboxylic acid group.

Suitable buffering agents include: acetic acid and salt (1-2% w/v); citric acid and a salt (1-3% w/v); boric acid and a salt (0.5-2:5% w/v); sodium bicarbonate (0.5-1.0% w/v); and phosphoric acid and a salt (0.8-2% w/v). Suitable preservatives include benzalkonium chloride (0.003-0.03% w/v); chlorbutanol (0.3-0.9% w/v); parabens (0.01-0.25% w/v) and thimerosal (0.004-0.02% w/v).

The term “carrier” as used herein, and described more fully below, means one or more solid or liquid filler, dilutant or encapsulating substances which are suitable for administration to a human or other mammal. The “carrier” can be an organic or inorganic ingredient, natural or synthetic, with which the active ingredient is combined to facilitate administration.

The components of the pharmaceutical compositions are capable of being commingled with the conjugates of the present invention, and with each other, in a manner such that there is no interaction which would substantially impair the desired pharmaceutical efficacy. In certain embodiments the components of aerosol formulations include solubilized active ingredients, and optionally antioxidants, solvent blends and propellants for solution formulations; micronized and suspended active ingredients, and optionally dispersing agents and propellants for suspension formulations.

The term “adjuvant” is intended to include any substance which is incorporated into or administered simultaneously with the antibodies or conjugates of the invention and which nonspecifically potentiates the immune response in the subject. Adjuvants include, without limitation, aluminum compounds, e.g., gels, aluminum hydroxide and aluminum phosphate, and Freund's complete or incomplete adjuvant (in which the conjugate is incorporated in the aqueous phase of a stabilized water in paraffin oil emulsion). The paraffin oil can be replaced with different types of oils, e.g., squalene or peanut oil. Other materials with adjuvant properties include BCG (attenuated Mycobacterium bovis), calcium phosphate, levamisole, isoprinosine, polyanions (e.g., poly A:U), leutinan, pertussis toxin, cholera toxin, lipid A, saponins and peptides, e.g., muramyl dipeptide. Rare earth salts, e.g., lanthanum and cerium, can also be used as adjuvants. The amount of adjuvants depends on the subject and the particular antibody or conjugate used and can be readily determined by one skilled in the art without undue experimentation.

Other supplementary immune potentiating agents, such as cytokines, can be delivered in conjunction with the antibodies or conjugates of the invention. In one embodiment, cytokines are administered separately from antibodies or conjugates of the invention in order to supplement treatment. In another embodiment, cytokines are administered conjugated to an FcRn binding partner. The cytokines contemplated are those that will enhance the beneficial effects that result from administering the antibodies or FcRn binding partner conjugates according to the invention. In certain embodiments the cytokines chosen from IFN-α, IFN-β, IFN-γ, IL-1, IL-2, and TNF-α. Other useful cytokines and related molecules are believed to be IL-3, IL-4, IL-5, IL-6, IL-7, IL-8, IL-9, IL-10, IL-11, IL-12, IL-13, IL-18, leukemia inhibitory factor, oncostatin-M, ciliary neurotrophic factor, growth hormone, prolactin, CD40 ligand, CD27 ligand, CD30 ligand, and TNF-β. Other cytokines known to modulate T-cell activity in a manner likely to be useful according to the invention are colony-stimulating factors and growth factors including granulocyte and/or granulocyte-macrophage colony-stimulating factors (CSF-1, G-CSF, and GM-CSF) and platelet-derived, epidermal, insulin-like, transforming and fibroblast growth factors. The selection of the particular cytokines will depend upon the particular modulation of the immune system that is desired. The activity of cytokines on particular cell types is known to those of ordinary skill in the art.

The precise amounts of the foregoing cytokines used in the invention will depend upon a variety of factors, including the antibody or conjugate selected, the dose amount and dose timing selected, the mode of administration, and characteristics of the subject. The precise amounts selected can be determined without undue experimentation, particularly since a threshold amount will be any amount which will enhance the desired immune response. Thus, it is believed that nanogram to milligram amounts of cytokines are useful, depending upon the mode of delivery, but that nanogram to microgram amounts are likely to be most useful because physiological levels of cytokines are correspondingly low.

The preparations of the invention are administered in effective amounts. An “effective amount” is that amount of a conjugate or antibody that will, alone or together with further doses, stimulate a response as desired. A “therapeutically effective amount” as used herein is that amount of a conjugate or antibody that will, alone or together with further doses, stimulate a therapeutic response as desired. In various embodiments this can involve the prevention, alleviation, or stabilization of signs or symptoms of a disease, disorder or condition of the subject.

The amount of antibodies and FcRn binding partner conjugates in all pharmaceutical preparations made in accordance with the present invention should be a therapeutically effective amount thereof which is also a medically acceptable amount thereof. Actual dosage levels of antibodies or FcRn binding partner conjugates in the pharmaceutical compositions of the present invention can be varied so as to obtain an amount of antibody or FcRn binding partner conjugates which is effective to achieve the desired therapeutic response for a particular patient, pharmaceutical composition of antibody or FcRn binding partner conjugates, and mode of administration, without being toxic to the patient.

The selected dosage level and frequency of administration of the antibodies and conjugates of the invention will depend upon a variety of factors, including the means of administration, the time of administration, the rates of excretion and metabolism of the therapeutic agent(s) including FcRn binding partner conjugates, the duration of the treatment, other drugs, compounds and/or materials used in combination with antibodies or FcRn binding partner conjugates, the age, sex, weight, condition, general health and prior medical history of the patient being treated, and like factors well known in the medical arts. For example, the dosage regimen is likely to vary with pregnant women, nursing mothers and children relative to healthy adults. The precise amounts selected can be determined without undue experimentation, particularly since a threshold amount will be any amount which will effect the desired therapeutic response. Thus, it is believed that nanogram to milligram amounts are useful, depending upon the particular therapeutic agent and the condition of the subject, but that nanogram to microgram amounts are likely to be most useful because physiological and pharmacological levels of therapeutic agents are correspondingly low.

In general it is believed that doses for central airway pulmonary administration of the conjugates of the invention will fall in the range 10 ng/kg to 500 μg/kg of body weight. For example, doses of 0.1-10 μg/kg are believed to be useful for IFN-α-Fc, and doses of 1-100 μg/kg are useful for EPO-Fc. In some instances doses of more than 25 mg can best be made in divided doses.

In general it is believed that doses for central airway pulmonary administration of antibodies will fall in the range 100 μg/kg to about 40 mg/kg of body weight.. For example, doses of 500 μg/kg are believed to be useful for HUMIRA™. In some instances doses of more than 25 mg can best be made in divided doses.

A physician having ordinary skill in the art can readily determine and prescribe the therapeutically effective amount of the pharmaceutical composition required. For example, the physician could start doses of FcRn binding partner conjugates employed in the pharmaceutical composition of the present invention at levels lower than that required to achieve the desired therapeutic effect and gradually increase the dosage until the desired effect is achieved.

Compositions can be conveniently presented in unit dosage form and may be prepared by any of the methods well known in the art of pharmacy. All methods include the step of bringing the conjugate into association with a carrier which constitutes one or more accessory ingredients. In general, the compositions are prepared by uniformly and intimately bringing the conjugate into association with a liquid carrier, a finely divided solid carrier, or both, and then, if necessary, shaping the product.

Delivery systems can include time-release, delayed release or sustained release delivery systems. Such systems can avoid repeated administrations of the conjugates of the invention, further increasing convenience to the subject and the physician. Many types of release delivery systems are available and known to those of ordinary skill in the art. They include polymer based systems such as polylactic and polyglycolic acid, polyanhydrides and polycaprolactone, wax coatings, and the like.

For administration by inhalation, the conjugate of the invention can be conveniently delivered in the form of an aerosol. As noted above, the aerosol can be generated from pressurized packs or inhalers with the use of a suitable propellant, e.g., chlorofluorocarbons, hydrochlorofluorocarbons, hydrofluorocarbons, and hydrocarbons including dichlorodifluoromethane, trichlorofluoromethane, dichlorotetrafluoroethane, 1,1,1,2-tetrafluoroethane, 1,1,1,2,3,3,3-heptafluoropropane, or other suitable propellant. In one embodiment, the aerosol is generated by contacting a solution or suspension containing the conjugate with a vibrational element such as a piezoelectric crystal connected to a suitable energy source. In certain embodiments the aerosol contains and delivers conjugates or antibodies substantially in their native, non-denatured form. In the case of a pressurized aerosol, the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The invention may be further understood with reference to the following examples, which are non-limiting.

### EXAMPLES

Materials. SATA, N-succinimdyl S-acetylthioacetate; sulfo-LC-SPDP, sulfosuccinimidyl 6-[3′-(2-pyridyldithio)-propionamido]hexanoate; and sulfo-SMCC, sulfosuccinimidyl 4-(N-maleimidomethyl) cyclohexane-1-carboxylate were purchased from Pierce (Rockford, Ill.). BALB/c mice were purchased from Charles River Laboratories (Wilmington, Mass.).

Enzymes and Cells. All restriction and modifying enzymes were purchased from New England Biolabs (Beverly, Mass.) or InVitrogen (GIBCO, Gaithersburg, Md.), and were used according to the manufacturers' protocols. Vent polymerase was obtained from New England Biolabs (Beverly, Mass.) and Expand polymerase from Roche Molecular Biochemicals (Indianapolis, Ind.), and both were used in their manufacturer-supplied buffers with magnesium. Shrimp alkaline phosphatase (SAP) was purchased from Roche Molecular Biochemicals (Indianapolis, Ind.). All oligonucleotides were synthesized and purified by Integrated DNA Technologies, Inc. (Coralville, Iowa). The DH5α competent cells were purchased from InVitrogen (GIBCO, Gaithersburg, Md.), and were used according to the manufacturer's protocol.

Expression Vector. The mammalian expression vector pED.dC was obtained from Genetics Institute (Cambridge, Mass.). This vector, derived from pED4 described in Kaufman R J et al. (1991) Nucleic Acids Res 19:4485-90, contains the adenovirus major late promoter, which is commonly used in expression vectors for efficient transcription, and an IgG intron for increased RNA stability and export. The vector also contains an adenovirus mRNA leader sequence, EMC virus 5′ UTR (ribosome entry sequence), SV40 polyA signal, and adenovirus stability element, to increase the level of RNA and thus lead to greater expression of the target protein. The vector also contains a colE1 origin of replication for growth in bacteria, as well as the β-lactamase gene for ampicillin selection in bacteria. Finally, the vector encodes a dicistronic message. The first cistron would be the target protein, while the second cistron is the mouse dihydrofolate reductase (dhfr) gene. The dhfr gene allows for selection and amplification of the dicistronic message in dhfr-deficient cell lines. Schimke R T (1984) Cell 37:705-13; Urlaub G et al (1986) Somat Cell Mol Genet 12:555-566.

DNA templates. The vector A₂E/X was kindly provided by H. Ploegh (Massachusetts Institute of Technology, Cambridge, Mass.), wt EPO-Fc was kindly provided by Wayne Lencer (Harvard Medical School, Boston, Mass.). Adult kidney cDNA was purchased from Clontech (Palo Alto, Calif.). The pGEM-T Easy vector was purchased from Promega (Madison, Wis.).

Oligonucleotide Primers. The following oligonucleotides (shown 5′ to 3′ from left to right) were used in the construction of the EPO-Fc expression vectors. The portion of each primer designed to anneal to the corresponding cDNA molecule or template is underlined.

| | |
|---|---|
| PKLF: | |
| aaaactgcagaccaccatggtaccgtgcacg | (SEQ ID NO:18) |
| KXR: | |
| cgtctagagccggcgcgggtctgagtcgg | (SEQ ID NO:19) |
| FCGF: | |
| aagaattcgccggcgccgctgcggtcgacaaaactc | (SEQ ID NO:20) |
| FCGMR: | |
| ttcaattgtcatttacccggagacaggg | (SEQ ID NO:21) |
| EPO-F: | |
| aatctagagccccaccacgcctcatctgtgac | (SEQ ID NO:22) |
| EPO-R: | |
| ttgaattctctgtcccctgtcctgcaggcc | (SEQ ID NO:23) |
| EPS-F: | |
| gtacctgcaggcggagatgggggtgca | (SEQ ID NO:24) |
| EPS-R: | |
| cctggtcatctgtcccctgtcc | (SEQ ID NO:25) |

PCR Amplification. Polymerase chain reactions were performed in either an Idaho Technology RapidCycler or MJ Research PTC-200 Peltier Thermal Cycler.

DNA Isolation and Purification. PCR products and all restriction enzyme digestions were electrophoresed and DNA bands corresponding to the correct size were excised from an agarose gel; DNA thus excised was purified using the Qiagen DNA Purification Kit (Valencia, Calif.) following the manufacturer's protocol. The 1 Kb DNA ladder or 1 Kb Plus DNA ladder from Life Technologies (Rockville, Md.) were used for determining the size of the DNA fragments. The concentration of the eluted DNA was estimated by visualization on an agarose gel or measurement of OD₂₆₀.

Ligation and Transformation. Ligation reactions were carried out using T4 DNA ligase (New England Biolabs, Beverly, Mass.) according to established protocols (Sambrook et. al (1989) Molecular Cloning: A Laboratory Manual, Second Edition, Cold Spring Harbor, N.Y.: Cold Spring Harbor Laboratory Press) or using the Rapid DNA Ligation Kit (Roche, Indianapolis, Ind.) according to the manufacturer's protocol. Ligation products were used for transformations of Escherichia coli strain DH5α according to established protocols. Sambrook et. al (1989) Molecular Cloning: A Laboratory Manual, Second Edition, Cold Spring Harbor, N.Y.: Cold Spring Harbor Laboratory Press.

DNA Sequencing. The sequence of the double-stranded plasmid DNA was determined by dideoxy sequencing performed at Dana Farber Molecular Biology Core Facilities (Boston, Mass.) or Veritas, Inc. (Rockville, Md.). The sequences were compiled using SeqMan (DNAStar, Madison, Wis.) and additional DNA analysis was performed using the LaserGene Suite of programs (DNAStar, Madison, Wis.) or Vector NTI (Informax, Gaithersburg, Md.).

Expression. Expression constructs were transfected into Chinese Hamster Ovary (CHO) dhfr-deficient (dhfr-) cell lines. Stable transfected cell lines were generated. In order to increase the EPO-Fc expression levels, the EPO-Fc gene was amplified by increasing the methotrexate concentration in the growth medium.

### Example 1

Preparation of Human Immunoglobulin G

In order to prepare human IgG or human IgG fragments for the use in conjugation to a compound of the invention, e.g., an antigen or therapeutic agent, the following methods may be used. Non-specific purified human IgG may be purchased from commercial vendors such as Sigma Chemical Co., Pierce Chemical, HyClone Laboratories, ICN Biomedicals, and Organon Teknika-Cappel.

Immunoglobulin G also may be isolated by ammonium sulfate precipitation of blood serum. The protein precipitate is further fractionated by ion exchange chromatography or gel filtration chromatography to isolate substantially purified non-specific IgG. By non-specific IgG it is meant that no single antigen specificity is dominant within the antibody population or pool.

Immunoglobulin G also may be purified from blood serum by adsorption to protein A attached to a solid support such as protein A-Sepharose (Pharmacia), AvidChrom-Protein A (Sigma), or protein G-Sepharose (Sigma). Other methods of purification of IgG are well known to persons skilled in the art and may be used for the purpose of isolation of non-specific IgG.

To prepare the Fc fragments of human IgG, isolated or purified IgG are subjected to digestion with immobilized papain (Pierce) according to the manufacturer's recommended protocol. Other proteases that digest IgG to produce intact Fc fragments that can bind to Fc receptors, e.g., plasmin (Sigma) or immobilized ficin (Pierce), are known to skilled artisans and may be used to prepare Fc fragments. The digested immunoglobulin then is incubated with an affinity matrix such as protein A-Sepharose or protein G-Sepharose. Non-binding portions of IgG are eluted from the affinity matrix by extensive washing In batch or column format. Fc fragments of IgG then are eluted by addition of a buffer that is incompatible with Fc-adsorbent binding. Other methodologies effective in the purification of Fc fragments also may be employed.

### Example 2

Conjugation of Compounds to Human Immunoglobulin Fc Fragments

To deliver compounds via the FcRn transport mechanism, such compounds can be coupled to whole IgG or Fc fragments. The chemistry of cross-linking and effective reagents for such purposes are well known in the art. The nature of the crosslinking reagent used to conjugate whole IgG or Fc fragments and the compound to be delivered is not restricted by the invention. Any crosslinking agent may be used provided that the activity of the compound is retained and binding by the FcRn of the Fc portion of the conjugate is not adversely affected.

An example of an effective one-step crosslinking of Fc and a compound is oxidation of Fc with sodium periodate in sodium phosphate buffer for 30 minutes at room temperature, followed by overnight incubation at 4° C. with the compound to be conjugated. Conjugation also may be performed by derivatizing both the compound and Fc fragments with sulfo-LC-SPDP for 18 hours at room temperature. Conjugates also may be prepared by derivatizing Fc fragments and the desired compound to be delivered with different crosslinking reagents that will subsequently form a covalent linkage. An example of this reaction is derivatization of Fc fragments with sulfo-SMCC and the compound to be conjugated to Fc is thiolated with SATA. The derivatized components are purified free of crosslinker and combined at room temperature for one hour to allow crosslinking. Other crosslinking reagents comprising aldehyde, imide, cyano, halogen, carboxyl, activated carboxyl, anhydride and maleimide functional groups are known to persons of ordinary skill in the art and also may be used for conjugation of compounds to Fc fragments. The choice of cross-linking reagent will, of course, depend on the nature of the compound desired to be conjugated to Fc. The crosslinking reagents described above are effective for protein-protein conjugations. If the compound to be conjugated is a carbohydrate or has a carbohydrate moiety, then heterobifunctional crosslinking reagents such as ABH, M2C2H, MPBH and PDPH are useful for conjugation with a proteinaceous FcRn-binding molecule (Pierce). Another method of conjugating proteins and carbohydrates is disclosed by Brumeanu et al. (Genetic Engineering News, Oct. 1, 1995, p. 16). If the compound to be conjugated is a lipid or has a lipid moiety which is convenient as a site of conjugation for the FcRn-binding molecule, then crosslinkers such as SPDP, SMPB and derivatives thereof may be used (Pierce). It is also possible to conjugate any molecule which is to be delivered by noncovalent means. One convenient way for achieving noncovalent conjugation is to raise antibodies to the compound to be delivered, such as monoclonal antibodies, by methods well known in the art, and select a monoclonal antibody having the correct Fc region and desired antigen binding properties. The antigen or therapeutic agent to be delivered is then prebound to the monoclonal antibody carrier. In all of the above crosslinking reactions it is important to purify the derivatized compounds free of crosslinking reagent. It is important also to purify the final conjugate substantially free of unconjugated reactants. Purification may be achieved by affinity, gel filtration or ion exchange chromatography based on the properties of either component of the conjugate. In one method an initial affinity purification step using protein A-Sepharose is used to retain Fc and Fc-compound conjugates, followed by gel filtration or ion exchange chromatography based on the mass, size or charge of the Fc conjugate. The initial step of this purification scheme ensures that the conjugate will bind to FcRn which is an essential requirement of the invention.

### Example 3

Construction of a General-Use X-Fc Expression Vector The K^{b} signal peptide allows for efficient production and secretion of many different possible proteins fused to Fcγ1. A general-use X-Fc expression vector was therefore constructed by inserting into the first cistron position of pED.dC an expression cassette consisting of the K^{b} signal peptide fused to aspartic acid 221 (D221, EU numbering) in the hinge region of Fcγ1 by a 13-amino acid peptide linker (GSRPGEFAGAAAV; SEQ ID NO: 26).

The K^{b} signal sequence was obtained from the A₂E/X template using primers PKF and KXR in the RapidCycler using Vent polymerase, denaturing at 95° C. for 15 sec, followed by 28 cycles with a slope of 6.0 of 95° C. for 0 sec, 55° C. for 0 sec, and 72° C. for followed by 3 min extension at 72° C. Primer PKF contains a PstII site, while primer KXR contains an XbaI site. The two restriction sites facilitated directional cloning of the amplified product. A PCR product of approximately 90 base pairs (bp) was gel purified, digested with PstI and XbaI, gel purified again and subcloned into a PstI/XbaI-digested, gel purified pED.dC vector. One construct was chosen as the representative clone and named pED.dC.K^{b}.

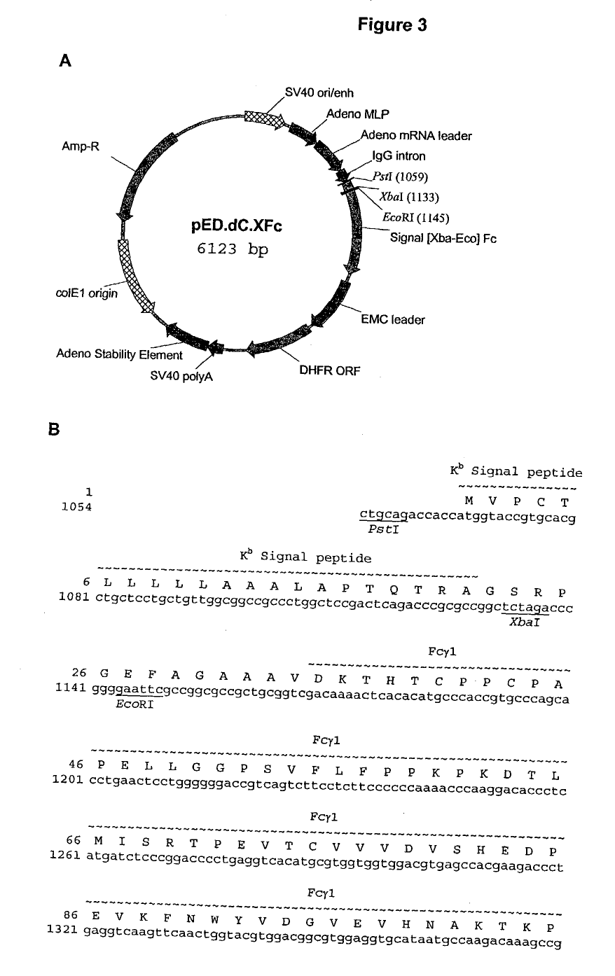

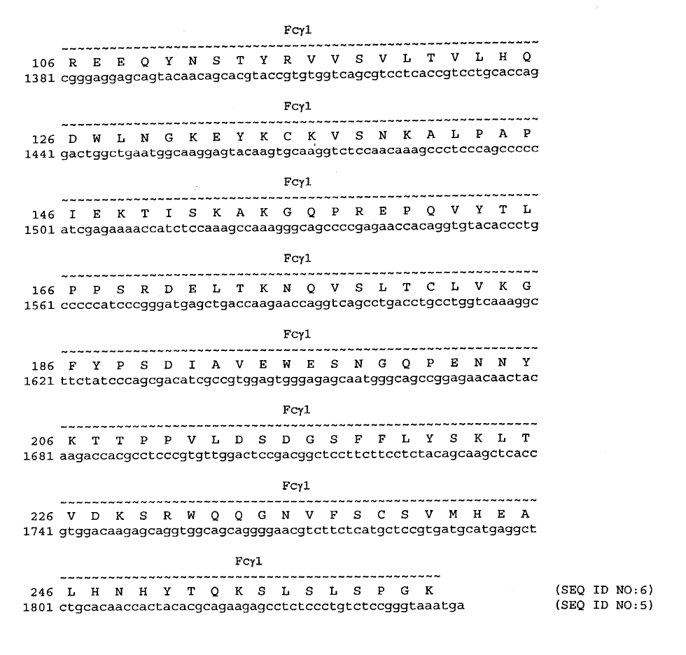

The Fcγ1 sequence was obtained from wt EPO-Fc template using primers FCGF and FCGMR in the RapidCycler using Expand polymerase, denaturing at 95° C. for 15 sec, followed by 30 cycles with a slope of 6.0 of 95° C. for 0 sec, 50° C. for 0 sec, and 72° C. for 1 min 20 sec, followed by 10 min extension at 72° C. A product of approximately 720 bp was gel-isolated and cloned into pGEM-T Easy vector and then sequenced. The correct coding region was then excised by EcoRI-MfeI digestion, gel purified and subcloned into the EcoRI-digested, gel-purified pED.dC.K^{b} construct. The plasmid with the Fcγ coding region in the correct orientation was determined by digestion with SmaI, and the sequence of this construct was determined. The construct was named pED.dC.XFc. The plasmid map and partial sequence of pED.dC.XFc is shown in FIG. 3.

### Example 4

Construction of an EPO-Fc Expression Vector with K^{b} Signal Peptide

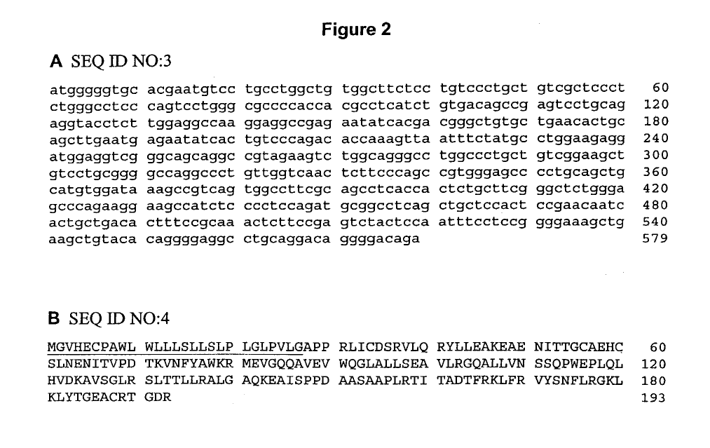

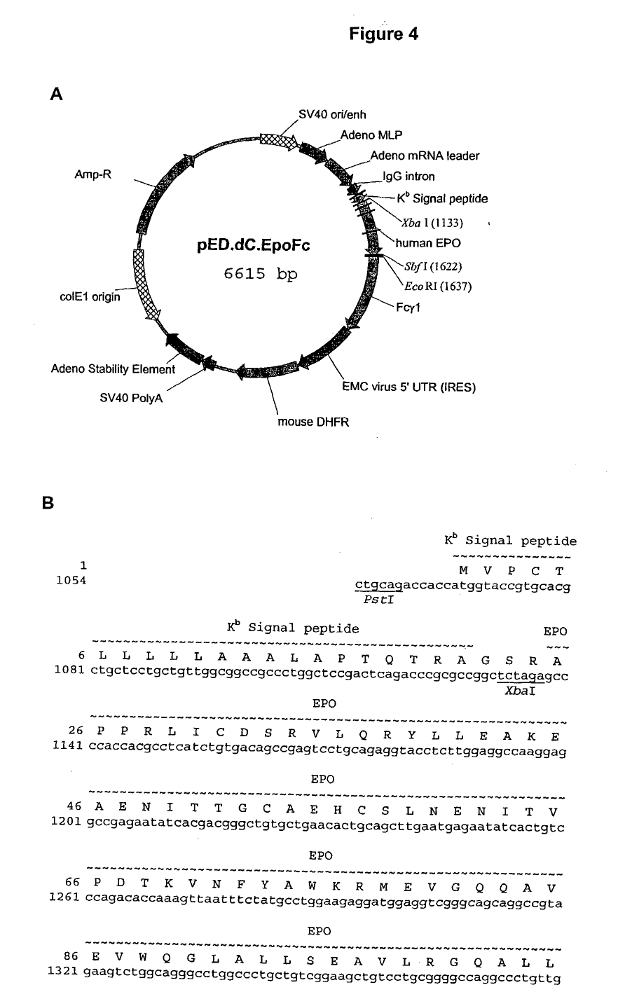

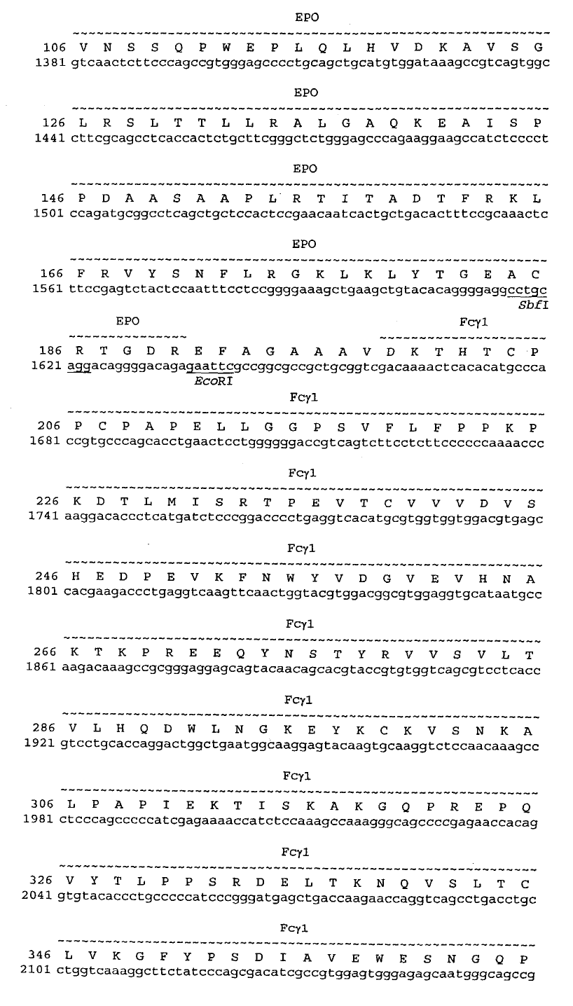

In this example, the mature human EPO sequence was inserted into the cassette, generating a cDNA encoding the K^{b} signal peptide, a 3-amino acid linker (GSR), the mature EPO sequence, and an 8-amino acid linker (EFAGAAAV, SEQ ID NO: 27), followed by the Fcγ1 sequence. The EPO sequence was obtained from an adult kidney QUICK-clone cDNA preparation as the template using primers EPO-F and EPO-R in the RapidCycler using Vent polymerase, denaturing at 95° C. for 15 sec, followed by 28 cycles with a slope of 6.0 of 95° C. for 0 sec, 55° C. for 0 sec, and 72° C. for 1 min 20 sec, followed by 3 min extension at 72° C. Primer EPO-F contains an XbaI site, while primer EPO-R contains an EcoRI site. An approximately 514 bp product was gel-purified, digested with XbaI and EcoRI, gel-purified again, and directionally subcloned into an XbaI/EcoRI-digested, gel-purified pED.dC.XFc vector. Following transformation, four of the twenty clones examined possessed the correct insert. One such clone was found to be free of mutations as determined by direct sequencing. This construct was named pED.dC.EpoFc. Refer to FIG. 2 for nucleic acid and amino acid sequences of wildtype human EPO. The plasmid map and partial sequence of pED.dC.EpoFc is shown in FIG. 4.

### Example 5

Construction of an EPO-Fc Expression Vector with EPO Signal Peptide

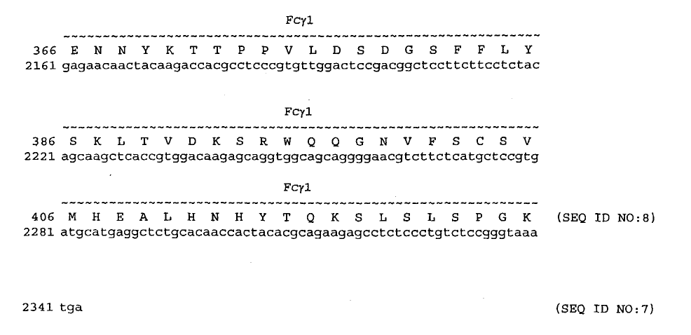

To evaluate the production and secretion of EPO-Fc when the endogenous EPO signal peptide was used rather than the K^{b} signal, a second EPO-Fc expression plasmid was generated. The secretion cassette in this plasmid encoded the human EPO sequence including its endogenous signal peptide fused to an 8-amino acid linker (EFAGAAAV, SEQ ID NO: 27), followed by the Fcγ1 sequence. The native EPO sequence, containing both the endogenous signal peptide and the mature sequence, was obtained from an adult kidney QUICK-clone cDNA preparation as the template using EPS-F and EPS-R primers in the PTC-200 using Expand polymerase, denaturing at 94° C. for 2 min, followed by 32 cycles of 94° C. for 30 sec, 57° C. for 30 sec, and 72° C. for 45 see, followed by 10 min extensions at 72° C. The primer EPS-F contains an SbfI site upstream of the start codon, while the primer EPS-R anneals downstream of the endogenous SbfI site in the EPO sequence. An approximately 603 bp product was gel-isolated and subcloned into the pGEM-T Easy vector. Four independent constructs were fully sequenced, and one of the two that were free of mutations was used for further subcloning. The correct coding sequence was excised by SbfI digestion, gel-purified, and cloned into the PstI-digested, SAP-treated, gel-purified pED.dC.EpoFc plasmid. The plasmid with the insert in the correct orientation was initially determined by KpnI digestion. A XmnI and PvuII digestion of this construct was compared with pED.dC.EpoFc and confirmed the correct orientation. The sequence was determined and the construct was named pED.dC.natEpoFc. The plasmid map and partial sequence of pED.dC.natEpoFc is shown in FIG. 5.

### Example 6

Retention of Biological Activity of EPO-Fc in vivo

In order to demonstrate that a conjugate made by the fusion of an FcRn binding partner and a protein of interest is capable of retaining biological activity, the example protein above was expressed and assayed for biological activity of erythropoietin in the following manner. The mammalian expression vector containing the EPO-Fc fusion was transfected into Chinese hamster ovary (CHO) cells and expressed by standard protocols in the art. Supernatants of transfected or non-transfected CHO cells were collected and injected subcutaneously into BALB/c mice. Reticulocyte counts of mice were obtained by Coulter FACS analysis by techniques known in the field of the art. Results demonstrated that mice injected with the supernatants of the transfected cells had reticulocyte counts several fold higher than mice injected with control (untransfected) supernatants. Since EPO has been documented to stimulate the production of erythrocytes, the results disclosed herein support the ability of the invention to synthesize biologically active FcRn binding partner conjugates.

Similarly, fusion proteins substituting the Fc fragment for an alternate FcRn binding partner domain in the vector described above would be expected to retain biological activity.

### Example 7

Transepithelial Absorption of EPO-Fc after Delivery to Central Airways

Immunohistochemical studies showed that FcRn is expressed at relatively higher levels in the central airways than in the alveolar epithelium in both cynomolgus monkeys and humans. Therefore, it was of interest to determine whether an EPO-Fc fusion protein (MW=112 kDa) that binds to FcRn can be transported through the lung epithelium and where in the lung this absorption occurs. A human EPO-Fc fusion protein, comprised of native human EPO fused at its carboxyl terminus to the amino terminus of the Fc domain of human IgG1, was expressed in CHO cells and purified from the cell culture medium using Protein A affinity chromatography. The purified human EPO-Fc fusion protein was biologically active in vitro. EPO-Fc bound to the EPO receptor (EpoR) with high affinity (K_{d}=0.25 nM vs. 0.2 nM for native huEPO) and stimulated the proliferation of TF-1 human erythroleukemia cells (ED₅₀=0.07 nM vs. 0.03 nM for native huEPO). EPO-Fc also bound to purified, soluble huFcRn (K_{d}=14 nM vs. 8 nM for IgG1) in a Biacore assay.

Aerosols of EPO-Fc(in PBS, pH 7.4) were created with various jet nebulizers and administered to anesthetized cynomolgus monkeys through endotracheal tubes. In some experiments monkeys were breathing spontaneously, while in other experiments the depth and rate of respiration were regulated with either a Bird Mark 7A respirator or a Spangler box apparatus. An increase in circulating reticulocytes was used as an indicator of the biological response to EPO-Fc. EPO-Fc was quantified in serum using a specific ELISA.

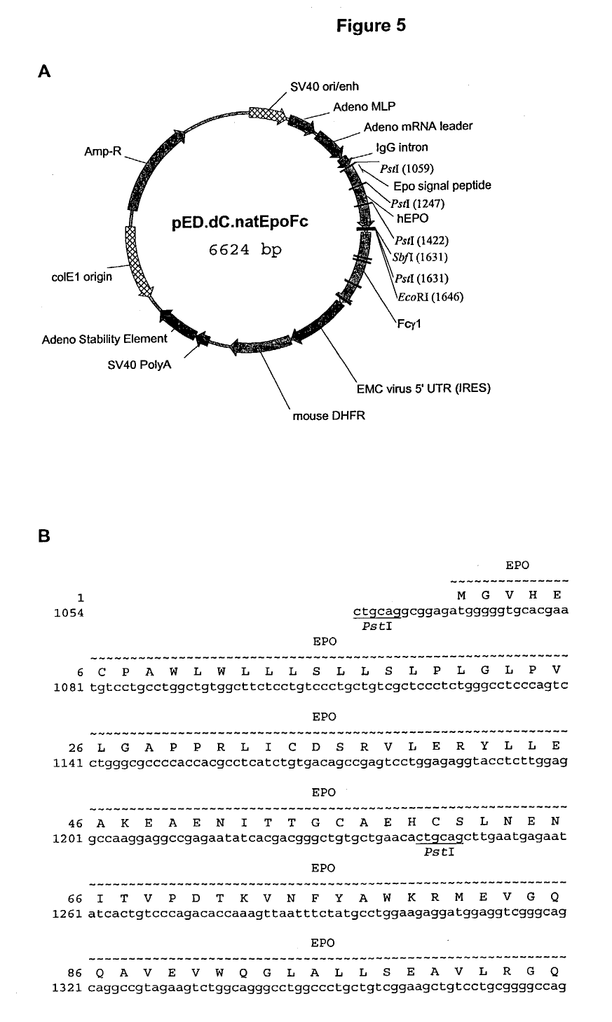

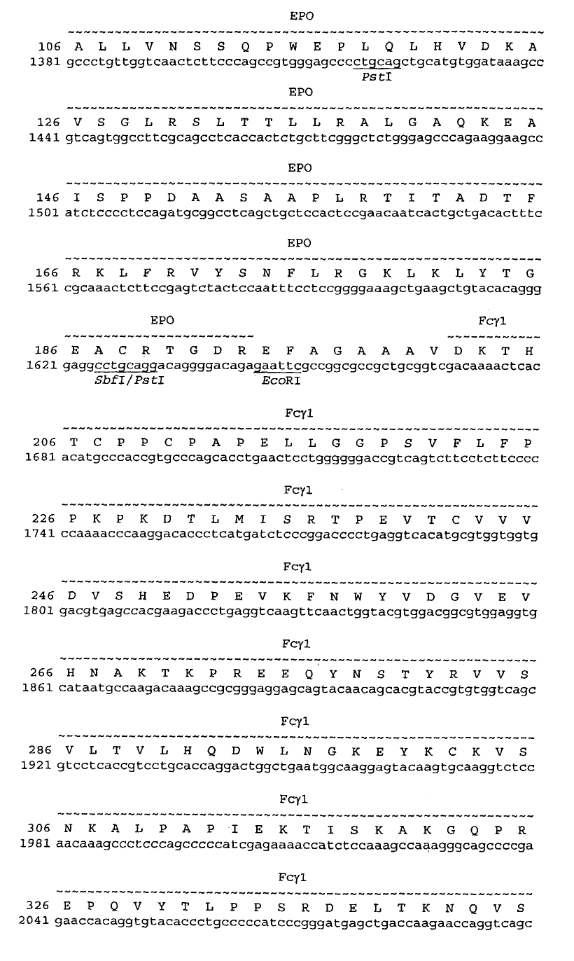

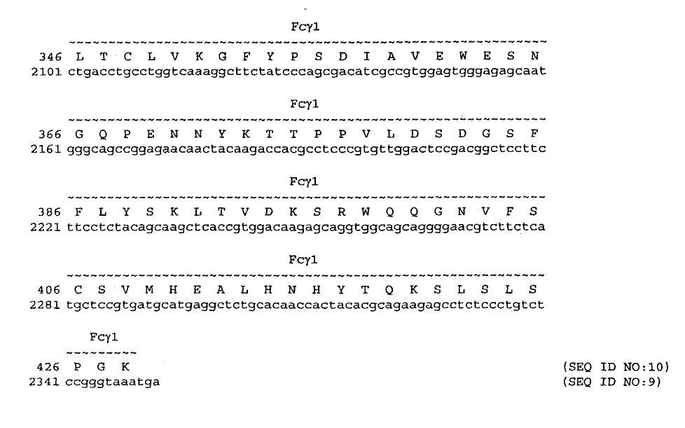

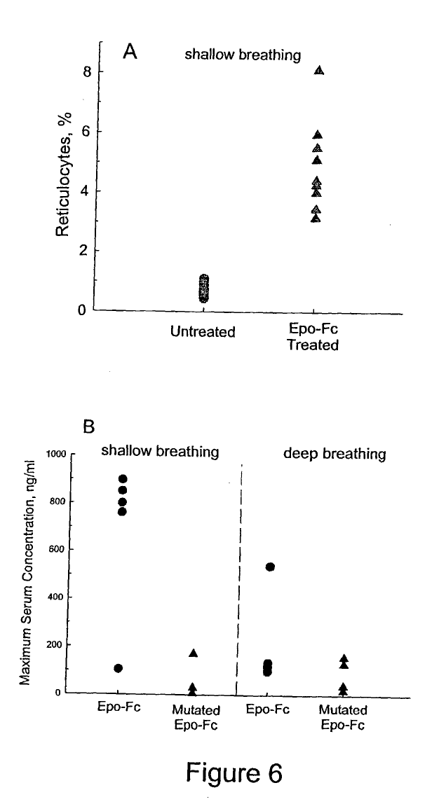

Initial studies in anesthetized, spontaneously breathing cynomolgus monkeys examined the biological response to aerosolized EPO-Fc (FIG. 6A). All animals in this study responded with an increase in circulating reticulocytes, 5-7 days after EPO-Fc administration. Subsequent studies showed that high concentrations of EPO-Fc were obtained in serum after single doses administered in a similar manner (FIG. 6B). A mutated EPO-Fc (Fc modified in three critical amino acid residues in the Fc domain: 1253A, H310A, and H435A) that is reduced in its FcRn binding by >90%, was not well absorbed. Mean serum half-life was approximately 22 hr for EPO-Fc(compared to 5-6 hr for EPOGEN® (Amgen)). The absorption of EPO-Fc and the mutEPO-Fc was compared using either shallow (spontaneous) breathing or deep (forced ventilation) breathing. Forced, deep breathing maneuvers resulted in much less absorption of EPO-Fc than shallow, spontaneous breathing, while there was no difference in absorption of mutated EPO-Fc.

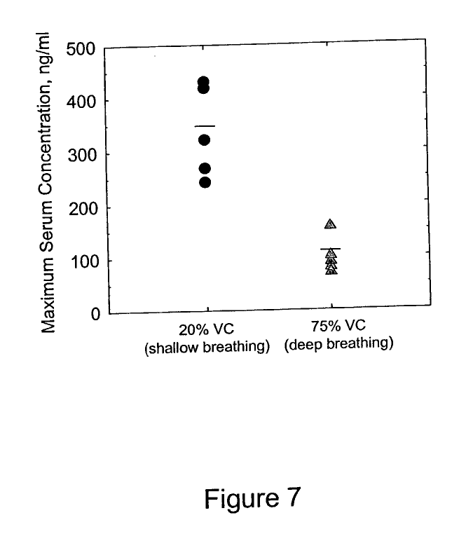

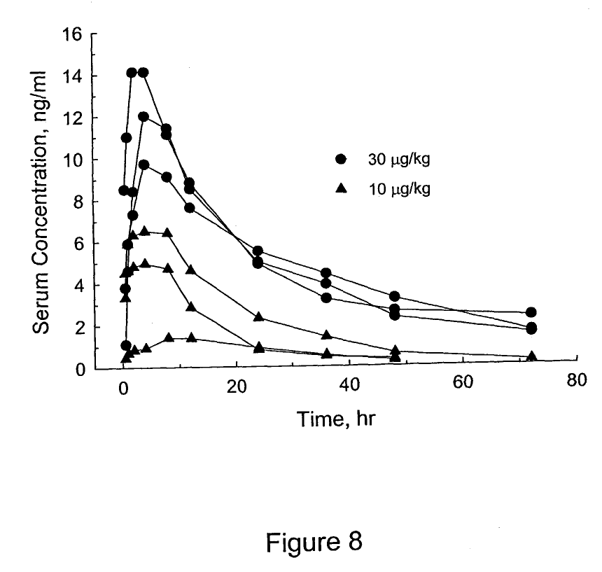

These results were confirmed and enhanced in an experiment using gamma scintigraphy (co-administration of ^{99m}Tc-DTPA as a radiotracer) to compare deposition and absorption of EPO-Fc with forced ventilation at either 20% or 75% vital capacity (FIG. 7). Scintigraphic images demonstrated that deposition of radiotracer was tracheal/central airway for 20% vital capacity vs. central airway/deep lung for 75% vital capacity. Absorption of EPO-Fc was more robust after administration using 20% of vital capacity. Additionally, the absorption of EPO-Fc was examined at different deposited dose levels (all done with 20% vital capacity maneuvers) to find a dose range for EPO-Fc that is clinically relevant. Deposited doses of 0.01-0.03 mg/kg resulted in pharmacokinetics consistent with clinical utility (FIG. 8).

### Example 8

Systemic Delivery of IFN-α by Aerosol Administration of Human IFN-α-Fc to Central Airways of Non-Human Primates

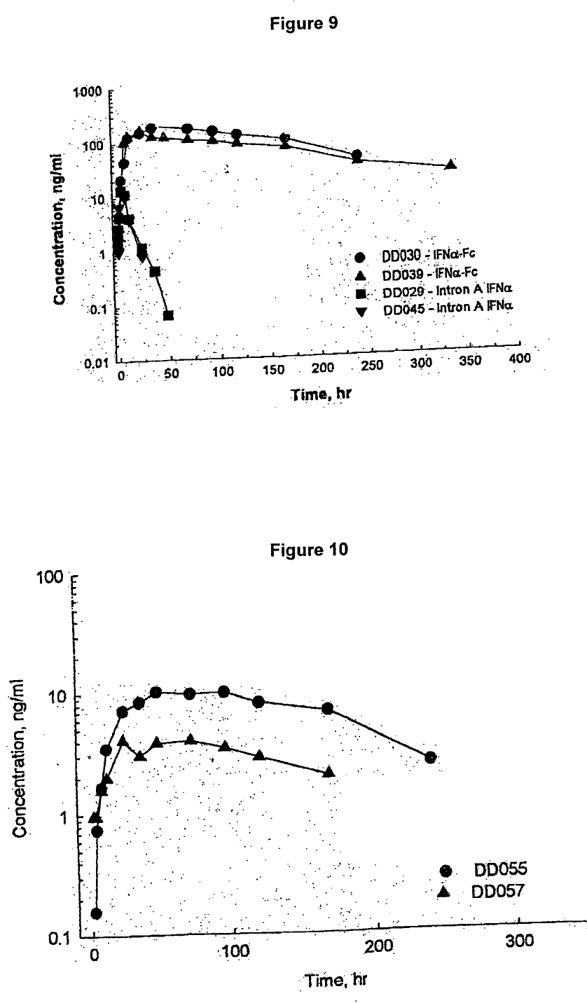

A human IFN-α-Fc expression construct was created using the pED.dC.K^{b} expression vector of Example 3 and the coding region of human IFN-α. The nucleotide sequence for human IFN-α is publicly available from GenBank as accession no. J00207. Human IFN-α-Fc was expressed in CHO cells and isolated in a manner analogous to that for EPO-Fc as described above. Six cynomolgus monkeys were divided into three groups for this experiment. Group I monkeys were administered 20 μg/kg of IFN-α-Fc by central airways aerosol administration analogous to the methods described for EPO-Fc administration in Example 7. Group II monkeys were administered 20 μg/kg of INTRON® A (Schering Corporation, Kenilworth, N.J.), recombinant human IFN-α, to central airways in the same manner. Group III monkeys were administered one tenth as much IFN-α-Fc as Group I, i.e., 2 μg/kg, by central airways aerosol administration. Blood samples were drawn periodically over 14 days and serum levels of IFN-α were determined at each time point using an appropriate specific ELISA. Pretreatment IFN-α levels, also determined by the same ELISA, were subtracted from all subsequent IFN-α level determinations. In addition, standard assays for bioactivity of IFN-α were performed using serial samples obtained from the animals in group I in order to assess bioactivity of the administered IFN-α-Fc. These assays included measurements of oligoadenylate synthetase (OAS) activity and of neopterin concentration. Results are shown in FIGS. 9-11.

FIG. 9 shows that monkeys in Group I (DD030 and DD039) achieved peak serum concentrations of IFN-α in the range of 160-185 ng/ml, with a half-life (T_{1/2}) of 83.7-109 hours. In contrast, monkeys in Group II (DD029 and DD045), receiving 20 μg/kg of IFN-α as INTRON® A in the same manner of administration, achieved peak serum levels of IFN-α of only about 13.6 ng/ml, with a half-life (T_{1/2}) of only 4.8-5.9 hours. These results indicate that aerosolized IFN-α-Fc administered to central airways is highly effective for systemic delivery of IFN-α. In addition, the prolonged half-life of IFN-α, thus adminsitered as IFN-α-Fc, demonstrates that IFN-α can be administered as an FcRn binding partner conjugate with dramatically improved pharmacokinetics compared to similarly administered IFN-α alone.

FIG. 10 shows that monkeys in Group III (DD055 and DD057), administered only on tenth as much IFN-α-Fc as monkeys in Group I, achieved proportionately lower serum concentrations with a similar pharmacokinetics profile.

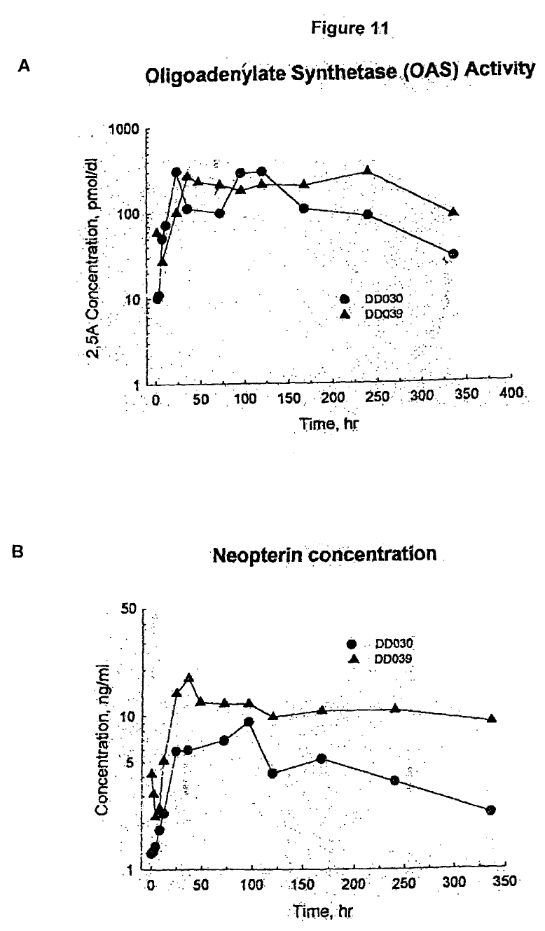

FIG. 11 shows the results of IFN-α bioactivity assays for Group I monkeys receiving IFN-α-Fc. FIG. 11A shows the increased and sustained OAS activity as a function of time paralleled the pharmacokinetic data in FIG. 9 and FIG. 10. FIG. 11B shows the increased and sustained neopterin concentration also paralleled the pharmacokinetic data in in FIG. 9 and FIG. 10. These data indicate that IFN-α in the IFN-α-Fc retains biological activity following aerosol administration to central airways according to the methods of the invention.

### Example 9

Systemic Delivery of TNFR-Fc by Aerosol Administration of Human TNFR-Fc to Central Airways of Non-Human Primates

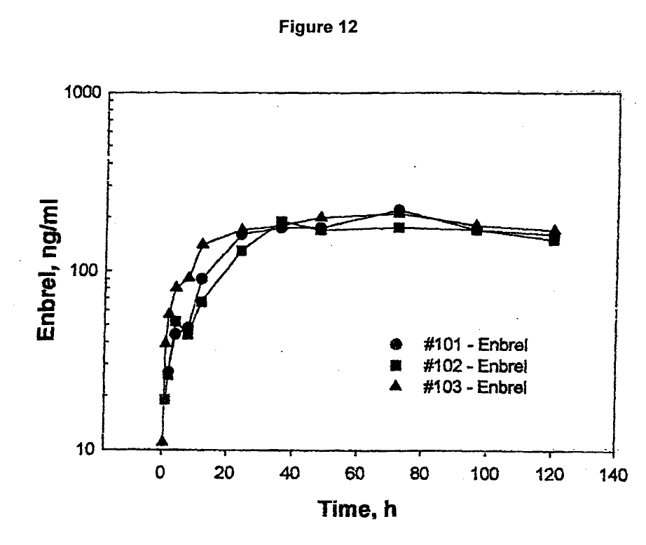

Each of three cynomolgus monkeys was administered aerosolized ENBREL® (etanercept, Immunex Corporation, Seattle, Wash.), recombinant human tumor necrosis factor receptor (TNFR)-Fcγ1, via the central airways according to the methods of the instant invention. ENBREL® is a dimeric fusion protein that includes the extracellular ligand-binding portion of human TNFR fused in frame to the hinge, C_{H}2, C_{H}3 domains of human IgG1. ENBREL® is expressed in CHO cells and has an approximate molecular weight of 150 kDa. The estimated deposited dose for each monkey in this experiment was 0.3-0.5 mg/kg. Blood samples were drawn periodically over ten days and serum levels of TNFR-Fc were determined at each time point using an appropriate specific ELISA. For the measurement of serum ENBREL® concentrations, a sandwich ELISA was performed using TNF-α bound to the plate as capture agent; serum or ENBREL® as the sample or standard, respectively; and anti-TNFR antibody as reporter agent. Results are shown in FIG. 12.

FIG. 12 shows that the three cynomolgus monkeys (101, 102, and 103) achieved similar peak serum concentrations of TNFR-Fc of about 200 ng/ml. The half-life of the TNFR-Fc was prolonged. This experiment demonstrates that human TNFR-Fc can be effectively administered to non-human primates via aerosol admininstration to the central airways according to the methods of the instant invention.

### Example 10

Systemic Delivery of IFN-β by Aerosol Administration of Human IFN-β-Fc to Central Airways of Non-Human Primates

A human IFN-β-Fc expression construct was created using the pED.dC.K^{b} expression vector of Example 3 and the coding region of human IFN-β. The nucleotide sequence for human IFN-β is publicly available from GenBank as accession no. V00535. Human IFN-β-Fc was expressed in CHO cells and isolated in a manner analogous to that for EPO-Fc as described above. Two cynomolgus monkeys and two rhesus monkeys each were administered 40 μg/kg of IFN-β-Fc by central airway aerosol administration analogous to the methods described for EPO-Fc administration in Example 7. Blood samples were drawn periodically over two days and serum levels of IFN-β were determined at each time point using an appropriate specific ELISA. Pretreatment IFN-β levels, also determined by the same ELISA, were subtracted from all subsequent IFN-β level determinations.

Results showed that both cynomolgus and rhesus monkeys administered aerosolized human IFN-β-Fc via the central airways achieved significant and sustained serum concentrations of IFN-β. The cynomolgus monkeys in this experiment achieved higher peak levels than did the rhesus monkeys (11.0-24.7 ng/ml for cynomolgus versus 5.4-8.4 ng/ml for rhesus). The half-life of IFN-β-Fc in both groups was about the same, i.e., 12.8-14.2 hours. These data demonstrate that aerosolized IFN-β-Fc administered to central airways of two species of non-human primates is effective for systemic delivery of IFN-β.

### Example 11

Systemic Delivery of FSH by Aerosol Administration of Human FSH-Fc to Central Airways of Non-Human Primates

A human FSH-Fc expression construct was created using the pED.dC.K^{b} expression vector of Example 3 and the coding region of a single-chain human FSH. The single chain FSH portion of the molecule includes both the α and the β chains of the heterodimeric hormone FSH, linked together in proper translational reading frame by a Sma I restriction endonuclease site (CCCGGG). The FSH-Fc construct is thus also referred to as hFSHβα-Fc. The nucleotide sequences for α and β subunits of human FSH are publicly available through GenBank as accession numbers NM_{—}000735 and NM_{—}000510, respectively. Human FSH-Fc was expressed in CHO cells and isolated in a manner analogous to that for EPO-Fc as described above.

Two cynomolgus monkeys were each administered 100 μg/kg of FSH-Fc by central airway aerosol administration analogous to the methods described for EPO-Fc administration in Example 7. Blood samples were drawn periodically over two weeks and serum levels of FSH were determined at each time point using appropriate specific ELISA. Pretreatment FSH levels, also determined by the same ELISA, were subtracted from all subsequent FSH level determinations. Results showed that both monkeys achieved significant levels of FSH, with peak serum concentrations of 21.6 and 42.8 ng/ml with a half-life of 145-153 hours.

### Example 12

Systemic Delivery of Monoclonal Anti-RSV Antibody by Aerosol Administration of SYNAGIS®0 to Central Airways of Non-Human Primates

In this example the humanized monoclonal anti-RSV antibody SYNAGIS® was shown to be systemically delivered to cynomolgus monkeys following central airway delivery according to the methods of the invention. The antibody was biotinylated in order to facilitate analysis.

Biotinylation of SYNAGIS® was performed with EZ-Link Sulfo-NHS-LC-Biotin or EZ-Link Sulfo-NHS-LC-LC-Biotin according to supplier's instructions with minor modification. EZ-Link Sulfo-NHS-LC-Biotin (Pierce, Cat #21335) and EZ-Link Sulfo-NHS-LC-LC-Biotin (Pierce, Cat #21338) are identical except that EZ-Link Sulfo-NHS-LC-LC-Biotin has an extra spacer LC. The first biotinylation of SYNAGIS(G) was done with EZ-Link Sulfo-NHS-LC-LC-Biotin. Subsequent biotinylation was done with EZ-Link Sulfo-NHS-LC-Biotin. The biotinylated SYNAGIS® was shown to have similar properties in terms of FcRn binding, uptake after oral administration in neonatal rats and detection in monkey serum.

SYNAGIS® was diluted to 10 mg/ml or 2 mg/ml in DPBS (without Calcium and Magnesium) in a microfuge tube. Immediately before use, 10 mM EZ-Link Sulfo-NHS-LC-Biotin or EZ-Link Sulfo-NHS-LC-LC-Biotin was made in distilled water. 27 μl of EZ-Link Sulfo-NHS-LC-Biotin or EZ-Link Sulfo-NHS-LC-LC-Biotin was added to 2 mg/ml antibody solution (20-fold molar ratio). 80 μl of EZ-Link Sulfo-NHS-LC-Biotin or EZ-Link Sulfo-NHS-LC-LC-Biotin was added to 10 mg/ml antibody solution (12-fold molar ratio). The tube was wrapped up in foil and placed in a slow rocker at room temperature for 30 minutes. The reaction was stopped by placing the tube on ice followed by dialysis in PBS overnight. The labeled SYNAGIS® was purified by Hi-trap protein A column. The molar ratio of biotin to SYNAGIS® was determined by HABA method supplied by the Manufacturer (Pierce) and was found to be approximately 1.5.

Cynomolgus monkeys were anesthetized and intubated with endotracheal tubes for delivery of aerosols directly into the lungs. Aerosols were generated with either an Aeroneb Pro nebulizer (Aerogen), mean mass aerodynamic diameter (MMAD) of approximately 4.5 μm for central airway delivery, or a Bird micronebulizer (Bird, MMAD of approximately 2.5 μm) for deep lung delivery. Each of the nebulizers was used in-line with a Bird Mark 7A respirator that was used to regulate breathing patterns. For central airway delivery the respirator was set to a pressure of approximately 10-15 cm H₂O and animals breathed 25-30 S breaths per minute (normal respiratory rate). For deep lung delivery the respirator was set to a pressure of 25-30 cm H₂O and animals breathed approximately 20 breaths per min, with approximately a 3 second breath hold between inspirations. SYNAGIS® was dissolved in phosphate-buffered saline. Two ml of aerosol was administered to each monkey, of which approximately 15% was deposited in the lungs and was available for absorption. The deposited dose in each monkey was approximately 0.6 mg/kg.

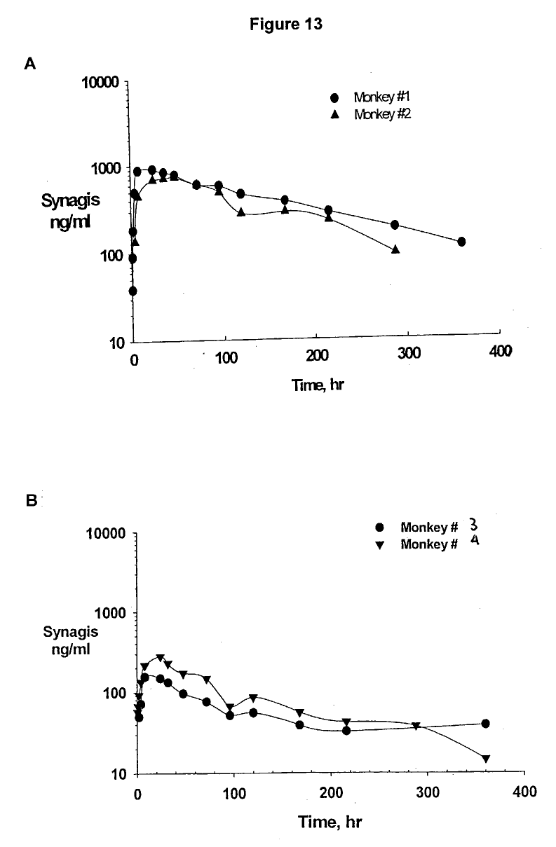

Serum samples were obtained and assayed for the biotinylated SYNAGIS®. Results are shown in FIG. 13. The monkeys in the central airway delivery group developed peak serum levels of SYNAGIS® of approximately 1000 ng/ml (FIG. 13A). In contrast, monkeys in the deep lung delivery group developed peak serum levels of SYNAGIS® of only approximately 200-300 ng/ml (FIG. 13B).

In a similar experiment, two cynomolgus monkeys each received a single 7 mg/kg deposited dose of biotinylated SYNAGIS® using the shallow breathing method described above. Peak serum concentrations of SYNAGIS(® were measured as 4 μg/ml.

The invention is not to be limited in scope by the specific embodiments described which are intended as single illustrations of individual aspects of the invention, and functionally equivalent methods and components are within the scope of the invention. Indeed various modifications of the invention, in addition to those shown and described herein, will become apparent to those skilled in the art from the foregoing description and accompanying drawings. Such modifications are intended to fall within the scope of the appended claims.

All references cited herein are incorporated herein in their entirety by reference for all purposes.

We claim:

1. A method for systemic delivery of a therapeutic agent, comprising:
administering an effective amount of an aerosol of a conjugate of a therapeutic agent and an FcRn binding partner to lung such that a central lung zone/peripheral lung zone deposition ratio (C/P ratio) is at least 0.7.

2. The method of claim 1, wherein the C/P ratio is at least 1.0.

3. The method of claim 1, wherein the C/P ratio is at least 1.5.

4. The method of claim 1, wherein the C/P ratio is at least 2.0.

5. The method of claim 1, wherein the therapeutic agent is a polypeptide.

6. The method of claim 1, wherein the therapeutic agent is an antigen.

7. The method of claim 6, wherein the antigen is a tumor antigen.

8. The method of claim 1, wherein the therapeutic agent is an oligonucleotide.

9. The method of claim 8, wherein the oligonucleotide is an antisense oligonucleotide.

10. The method of claim 1, wherein the therapeutic agent is erythropoietin (EPO), growth hormone, interferon alpha (IFN-α), interferon beta (IFN-β), or follicle stimulating hormone (FSH):

11. The method of claim 1, wherein the therapeutic agent is EPO.

12. A method for systemic delivery of a therapeutic agent, comprising:
administering an effective amount of an aerosol of a conjugate of a therapeutic agent and an FcRn binding partner to lung, wherein particles in the aerosol have a mass median aerodynamic diameter (MMAD) of at least 3 micrometers (μm).

13. The method of claim 12, wherein the MMAD of the particles is between 3 μm and about 8 μm.

14. The method of claim 12, wherein the MMAD of the particles is greater than 4 μm.

15. The method of claim 12, wherein a majority of the particles are non-respirable.

16. The method of claim 12, wherein the therapeutic agent is a polypeptide.

17. The method of claim 12, wherein the therapeutic agent is an antigen.

18. The method of claim 17, wherein the antigen is a tumor antigen.

19. The method of claim 12, wherein the therapeutic agent is an oligonucleotide.

20. The method of claim 19, wherein the oligonucleotide is an antisense oligonucleotide.

21. The method of claim 12, wherein the therapeutic agent is EPO, growth hormone, IFN-α, IFN-β, or FSH.

22. The method of claim 12, wherein the therapeutic agent is EPO.

23. An aerosol of a conjugate of a therapeutic agent and an FcRn binding partner, wherein particles in the aerosol have a MMAD of at least 3 μm.

24. The aerosol of claim 23, wherein the MMAD of the particles is between 3 μm and about 8 μm.

25. The aerosol of claim 23, wherein the MMAD of the particles is greater than 4 μm.

26. The aerosol of claim 23, wherein a majority of the particles are non-respirable.

27. The aerosol of claim 23, wherein the therapeutic agent is a polypeptide.

28. The aerosol of claim 23, wherein the therapeutic agent is an antigen.

29. The aerosol of claim 28, wherein the antigen is a tumor antigen.

30. The aerosol of claim 23, wherein the therapeutic agent is an oligonucleotide.

31. The aerosol of claim 30, wherein the oligonucleotide is an antisense oligonucleotide.

32. The aerosol of claim 23, wherein the therapeutic agent is EPO, growth hormone, IFN-α, IFN-β, or FSH.

33. The aerosol of claim 23, wherein the therapeutic agent is EPO.

34. An aerosol delivery system, comprising a container, an aerosol generator connected to the container, and a conjugate of a therapeutic agent and an FcRn binding partner disposed within the container, wherein the aerosol generator is constructed and arranged to generate an aerosol of the conjugate having particles with a MMAD of at least 3 μm.

35. The aerosol delivery system of claim 34, wherein the MMAD of the particles is greater than 4 μm.

36. The aerosol delivery system of claim 34, wherein a majority of the particles are non-respirable.

37. The aerosol delivery system of claim 34, wherein the aerosol generator comprises a vibrational element in fluid connection with a solution containing the conjugate.

38. The aerosol delivery system of claim 34, wherein the aerosol generator is a nebulizer.

39. The aerosol delivery system of claim 34, wherein the aerosol generator is a mechanical pump.

40. The aerosol delivery system of claim 34, wherein the container is a pressurized container.

41. A method of manufacturing the aerosol delivery system of claim 34, comprising:
providing the container;
providing the aerosol generator connected to the container; and
placing an effective amount of the conjugate in the container.

42. The method of claim 41, wherein the the aerosol generator comprises a vibrational element in fluid connection with a solution containing the conjugate.

43. The method of claim 41, wherein the aerosol generator is a nebulizer

44. The method of claim 41, wherein the aerosol generator is a mechanical pump.

45. The method of claim 41, wherein the container is a pressurized container.

46. A method for systemic delivery of an antibody to a subject, comprising:
administering to a central airway of a subject an antibody in an aerosol, wherein a central lung zone/peripheral lung zone deposition ratio (C/P ratio) is at least 0.7, in an effective amount to achieve systemic delivery of the antibody to the subject.

47. The method of claim 46, wherein the C/P ratio is at least 1.0.

48. The method of claim 46, wherein the C/P ratio is at least 1.5.

49. The method of claim 46, wherein the C/P ratio is at least 2.0.

50. The method of claim 46, wherein the systemic delivery is a peak serum concentration of the antibody of at least 0.5 μg/ml.

51. The method of claim 46, wherein the antibody comprises an FcRn binding domain.

52. The method of claim 46, wherein the antibody comprises a human Fc fragment.

53. The method of claim 46, wherein the antibody comprises a human IgG1 Fc fragment.

54. The method of claim 46, wherein the antibody is a monoclonal antibody.

55. The method of claim 46, wherein the antibody is an immune globulin or a hyperimmune globulin.

56. The method of claim 46, wherein the antibody is a therapeutic antibody.

57. The method of claim 56, wherein the therapeutic antibody is chosen from anti-CD52, anti-CD25, anti-TNF-α, anti-RSV, anti-CD20, anti-HER2, anti-CEA.

58. The method of claim 57, wherein the antibody is chosen from CAMPATH®, SIMULECT®, ZENAPAX®, REMICADE®, HUMIRA™, SYNAGIS®, RITUXAN®, HERCEPTIN®, and CEA-CIDE™.

59. The method of claim 57, wherein the antibody is CAMPATH®.

60. The method of claim 57, wherein the antibody is SIMULECT®.

61. The method of claim 57, wherein the antibody is ZENAPAX®.

62. The method of claim 57, wherein the antibody is REMICADE®.

63. The method of claim 57, wherein the antibody is HUMIRA™.

64. The method of claim 57, wherein the antibody is SYNAGIS®.

65. The method of claim 57, wherein the antibody is RITUXAN®.

66. The method of claim 57, wherein the antibody is HERCEPTIN®.

67. The method of claim 57, wherein the antibody is CEA-CIDE™.

68. The method of claim 46, wherein the antibody is a diagnostic antibody.

69. The method of claim 46, wherein the administering to a central airway of the subject comprises tidal breathing by the subject.

70. The method of claim 46, wherein a majority of particles of the aerosol are non-respirable particles.

71. A method for passively immunizing a subject, comprising:
administering to a central airway of a subject, wherein said subject is in need of passive immunization against an antigen, an antigen-specific antibody in an aerosol, wherein a central lung zone/peripheral lung zone deposition ratio (C/P ratio) is at least 0.7, in an effective amount to neutralize the antigen in the subject.

72. The method of claim 71, wherein the C/P ratio is at least 1.0.

73. The method of claim 71, wherein the C/P ratio is at least 1.5.

74. The method of claim 71, wherein the C/P ratio is at least 2.0.

75. The method of claim 71, wherein the antibody comprises an FcRn binding domain.

76. The method of claim 71, wherein the antibody comprises a human Fc fragment.

77. The method of claim 71, wherein the antibody comprises a human IgG1 Fc fragment.

78. The method of claim 71, wherein the antibody is a monoclonal antibody.

79. The method of claim 71, wherein the antibody is an immune globulin or a hyperimmune globulin.

80. The method of claim 71, wherein the administering to a central airway of the subject comprises tidal breathing by the subject.

81. The method of claim 71, wherein the aerosol comprises predominantly non-respirable particles.

82. A method for treating a deep lung disease in a subject, comprising:
administering to a central airway of a subject, wherein said subject is in need of an antibody for treatment of a deep lung disease, an antibody in an aerosol, wherein a central lung zone/peripheral lung zone deposition ratio (C/P ratio) is at least 0.7, in an effective amount to treat the deep lung disease of the subject.

83. The method of claim 82, wherein the C/P ratio is at least 1.0.

84. The method of claim 82, wherein the C/P ratio is at least 1.5.

85. The method of claim 82, wherein the C/P ratio is at least 2.0.

86. The method of claim 82, wherein the antibody comprises an FcRn binding domain.

87. The method of claim 82, wherein the antibody comprises a human Fc fragment.

88. The method of claim 82, wherein the antibody comprises a human IgG1 Fc fragment.

89. The method of claim 82, wherein the antibody is a monoclonal antibody.

90. The method of claim 82, wherein the antibody is an immune globulin or a hyperimmune globulin.

91. The method of claim 82, wherein the administering to a central airway of the subject comprises tidal breathing by the subject.

92. The method of claim 82, wherein the aerosol comprises predominantly non-respirable particles.

93. The method of claim 82, wherein the deep lung disease is chosen from RSV pneumonia, CMV pneumonia, primary lung cancer, extranodal pulmonary non-Hodgkin's lymphoma, and cancer metastatic to lung.

94. The method of claim 82, wherein the antibody is chosen from anti-RSV, anti-CMV, anti-CD52, anti-CD20, anti-HER2, and anti-CEA.

95. The method of claim 82, wherein the antibody is chosen from SYNAGIS®, CAMPATH®, RITUXAN®, HERCEPTIN®, and CEA-CIDE™.

96. The method of claim 82, wherein the antibody is SYNAGIS®.

97. The method of claim 82, wherein the antibody is CAMPATH®.

98. The method of claim 82, wherein the antibody is RITUXAN®.

99. The method of claim 82, wherein the antibody is HERCEPTIN®.

100. The method of claim 82, wherein the antibody is CEA-CIDE™.

101. A method for treating extrapulmonary disease in a subject, comprising:
administering to a central airway of a subject, wherein said subject is in need of an antibody for treatment of extrapulmonary disease, an antibody in an aerosol, wherein a central lung zone/peripheral lung zone deposition ratio (C/P ratio) is at least 0.7, in an effective amount to treat the extrapulmonary disease of the subject.

102. The method of claim 96, wherein the C/P ratio is at least 1.0.

103. The method of claim 96, wherein the C/P ratio is at least 1.5.

104. The method of claim 96, wherein the C/P ratio is at least 2.0.

105. The method of claim 96, wherein the antibody comprises an FcRn binding domain.

106. The method of claim 96, wherein the antibody comprises a human Fc fragment.

107. The method of claim 96, wherein the antibody comprises a human IgG1 Fc fragment.

108. The method of claim 96, wherein the antibody is a monoclonal antibody.

109. The method of claim 96, wherein the administering to a central airway of the subject comprises tidal breathing by the subject.

110. The method of claim 96, wherein the aerosol comprises predominantly non-respirable particles.

111. The method of claim 96, wherein the extrapulmonary disease is cancer.

112. The method of claim 111, wherein the antibody is chosen from anti-CD52, anti-CD25, anti-CD20, anti-HER2, and anti-CEA.

113. The method of claim 111, wherein the antibody is chosen from CAMPATH®, SIMULECT®, ZENAPAX®, RITUXAN®, HERCEPTIN®, and CEA-CIDE™.

114. The method of claim 111, wherein the antibody is CAMPATH®.

115. The method of claim 111, wherein the antibody is SIMULECT®.

116. The method of claim 111, wherein the antibody is ZENAPAX®.

117. The method of claim 111, wherein the antibody is RITUXAN®.

118. The method of claim 111, wherein the antibody is HERCEPTIN®.

119. The method of claim 111, wherein the antibody is CEA-CIDE™.

120. The method of claim 96, wherein the extrapulmonary disease is an autoimmune disease.

121. The method of claim 120, wherein the autoimmune disease is chosen from rheumatoid arthritis and Crohn's disease.

122. The method of claim 120, wherein the antibody is anti-TNF-α.

123. The method of claim 122, wherein the antibody is REMICADE®.

124. The method of claim 122, wherein the antibody is HUMIRA™.

125. The method of claim 96, wherein the extrapulmonary disease is non-pulmonary allograft rejection.

126. The method of claim 125, wherein the antibody is anti-CD25.

127. The method of claim 126, wherein the antibody is selected from SIMULECT® and ZENAPAX®.
